SEQUENCE LISTING (199 sequences)

SEQ ID NO: 1 - protein, 42 aa, Unknown [sequence omitted]

SEQ ID NO: 2 - protein, 8 aa, Artificial Sequence
  Binding Epitope
DAEFRHDS

SEQ ID NO: 3 - protein, 43 aa, Unknown
  Mammalia (Abeta1-43)
DAEFRHDSGYEVHHQKLVFFAEDVGSNKGAIIGLMVGGVVIAT

SEQ ID NO: 4 - protein, 41 aa, Unknown
  Mammalia
DAEFRHDSGYEVHHQKLVFFAEDVGSNKGAIIGLMVGGVVI

SEQ ID NO: 5 - protein, 40 aa, Unknown
  Mammalia (Abeta 1-40)
DAEFRHDSGYEVHHQKLVFFAEDVGSNKGAIIGLMVGGVV

SEQ ID NO: 6 - protein, 39 aa, Unknown
  Mammalia (Abeta 1-39)
DAEFRHDSGYEVHHQKLVFFAEDVGSNKGAIIGLMVGGV

SEQ ID NO: 7 - protein, 5 aa, Artificial Sequence
  Binding Epitope
DAEFR

SEQ ID NO: 8 - protein, 9 aa, Artificial Sequence
  Binding epitope
SNKGAIIGL

SEQ ID NO: 9 - protein, 7 aa, Artificial Sequence
  Binding epitope
LVFFAED

SEQ ID NO: 10 - protein, 121 aa, Artificial Sequence
  VH
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRRNGYDGGFAYWGQGTLVTVSA

SEQ ID NO: 11 - protein, 7 aa, Artificial Sequence
  VH CDR1
TSGMGVS

SEQ ID NO: 12 - protein, 16 aa, Artificial Sequence
  VH-CDR2
HIYWDDDKRYNPSLKS

SEQ ID NO: 13 - protein, 11 aa, Artificial Sequence
  VH-CDR3
RRNGYDGGFAY

SEQ ID NO: 14 - protein, 112 aa, Artificial Sequence
  VL
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELK

SEQ ID NO: 15 - protein, 16 aa, Artificial Sequence
  VL-CDR1
RSSQSIVHSNGNTYLE

SEQ ID NO: 16 - protein, 7 aa, Artificial Sequence
  VL-CDR2
KVSNRFS

SEQ ID NO: 17 - protein, 9 aa, Artificial Sequence
  VL-CDR3
FQGSHVPLT

SEQ ID NO: 18 - DNA, 363 bp, Artificial Sequence
  VH
caggttactctgaaagagtctggccctgggatattgcagtcctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagaaaccaggtattcctcaagatcaccagtgtggacactgcagatactgccacatactactgtgctcgaaggaggaatggttacgacggggggtttgcttactggggccaagggactctggtcactgtctctgca

SEQ ID NO: 19 - DNA, 336 bp, Artificial Sequence
  VL
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 20 - protein, 457 aa, Artificial Sequence
  ACI-24-41F12-Ab2 (IgG2b variant) HC
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRRNGYDGGFAYWGQGTLVTVSAAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 21 - protein, 219 aa, Artificial Sequence
  ACI-24-41F12-Ab2 (IgG2b Variant) LC
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 22 - protein, 451 aa, Artificial Sequence
  ACI-24-41F12-Ab3 (IgG2a variant) HC
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRRNGYDGGFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - protein, 219 aa, Artificial Sequence; identical to SEQ ID NO: 21
  ACI-24-41F12-Ab3 (IgG2a variant) LC

SEQ ID NO: 24 - protein, 451 aa, Artificial Sequence
  ACI-24-41F12-Ab3 (hIgG1 variant) HC
QVTLKESGPGILQSSQTLSLTCSFSGFSLSTSGMGVSWIRQPSGKGLEWLAHIYWDDDKRYNPSLKSRLTISKDTSRNQVFLKITSVDTADTATYYCARRRNGYDGGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 219 aa, Artificial Sequence
  ACI-24-41F12-Ab3 (hIgG1 variant) LC
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - skipped

SEQ ID NO: 27 - protein, 458 aa, Artificial Sequence
  ACI-31-25B1-Ab2 (murine IgG2b with a lambda LC) HC
EVQLVESGGDLVKPGGSLKLSCAASRFTFSTYGMSWVRQTPDKRLEWVATISSGRSYTYYADSVKGRFTISRDNAKNTLYLQMSSLKSEDTAMYYCARNSNNQRDYYAMDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 28 - protein, 221 aa, Artificial Sequence
  ACI-31-25B1-Ab2 (murine IgG2b with a lambda LC) LC
QLVLTQSSSASFSLGASAKLTCTLSSQHITYTIEWYQQQPLKPPKYVMVLEKDGSHSTGDGIPDRFSGSSSGADRYLSISNIQPEDEAVYICGVGDAIKEQFVYVFGGGTKVTVLGQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

SEQ ID NO: 29 - protein, 14 aa, Artificial Sequence
  Binding epitope
EDVGSNKGAIIGLM

SEQ ID NO: 30 - protein, 122 aa, Artificial Sequence
  ACI-31-25B1-Ab2 HCVR
EVQLVESGGDLVKPGGSLKLSCAASRFTFSTYGMSWVRQTPDKRLEWVATISSGRSYTYYADSVKGRFTISRDNAKNTLYLQMSSLKSEDTAMYYCARNSNNQRDYYAMDYWGQGTSVTVSS

SEQ ID NO: 31 - protein, 5 aa, Artificial Sequence
  ACI-31-25B1-Ab2 VH-CDR1
TYGMS

SEQ ID NO: 32 - protein, 17 aa, Artificial Sequence
  ACI-31-25B1-Ab2 VH-CDR2
TISSGRSYTYYADSVKG

SEQ ID NO: 33 - protein, 13 aa, Artificial Sequence
  ACI-31-25B1-Ab2 VH-CDR3
NSNNQRDYYAMDY

SEQ ID NO: 34 - protein, 115 aa, Artificial Sequence
  ACI-31-25B1-Ab2 LCVR
QLVLTQSSSASFSLGASAKLTCTLSSQHITYTIEWYQQQPLKPPKYVMVLEKDGSHSTGDGIPDRFSGSSSGADRYLSISNIQPEDEAVYICGVGDAIKEQFVYVFGGGTKVTVL

SEQ ID NO: 35 - protein, 12 aa, Artificial Sequence
  ACI-31-25B1-Ab2 VL-CDR1
TLSSQHITYTIE

SEQ ID NO: 36 - protein, 7 aa, Artificial Sequence
  ACI-31-25B1-Ab2 VL-CDR2
GSHSTGD

SEQ ID NO: 37 - protein, 13 aa, Artificial Sequence
  ACI-31-25B1-Ab2 VL-CDR3
GVGDAIKEQFVYV

SEQ ID NO: 38 - protein, 452 aa, Artificial Sequence
  ACI-31-25B1-Ab2 (murine IgG2a with a lambda LC) HC
EVQLVESGGDLVKPGGSLKLSCAASRFTFSTYGMSWVRQTPDKRLEWVATISSGRSYTYYADSVKGRFTISRDNAKNTLYLQMSSLKSEDTAMYYCARNSNNQRDYYAMDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 39 - protein, 221 aa, Artificial Sequence; identical to SEQ ID NO: 28
  ACI-31-25B1-Ab2 (murine IgG2a with a lambda LC) LC

SEQ ID NO: 40 - protein, 117 aa, Artificial Sequence
  ACI-31-30C11-Ab1 HCVR
QVQLQQSGPELVKPGASVKISCKASGYAFSSSWLNWVKQRPGEGLEWIGRIYPGDGDINYNGKFKGKATLTADKSSSTAYMQLSSLTSDDSAVYFCTKRGRYALDYWGQGTSVTVSS

SEQ ID NO: 41 - protein, 5 aa, Artificial Sequence
  ACI-31-30C11-Ab1 VH-CDR1
SSWLN

SEQ ID NO: 42 - protein, 17 aa, Artificial Sequence
  ACI-31-30C11-Ab1 VH-CDR2
RIYPGDGDINYNGKFKG

SEQ ID NO: 43 - protein, 8 aa, Artificial Sequence
  ACI-31-30C11-Ab1 VH-CDR3
RGRYALDY

SEQ ID NO: 44 - protein, 111 aa, Artificial Sequence
  ACI-31-30C11-Ab1 LCVR
DIVLTQSPASLAVSLGQRATISCRASKSVDNYGKSFMHWFQQKPGQPPKLLIYRASNLESGIPARFSGSGSRTDFTLTINPVEADDVATYYCQQSNEDPYTFGGGTKLEMK

SEQ ID NO: 45 - protein, 15 aa, Artificial Sequence
  ACI-31-30C11-Ab1 VL-CDR1
RASKSVDNYGKSFMH

SEQ ID NO: 46 - protein, 7 aa, Artificial Sequence
  ACI-31-30C11-Ab1 VL-CDR2
RASNLES

SEQ ID NO: 47 - protein, 9 aa, Artificial Sequence
  ACI-31-30C11-Ab1 VL-CDR3
QQSNEDPYT

SEQ ID NO: 48 - protein, 453 aa, Artificial Sequence
  ACI-31-30C11-Ab1 (murine IgG2b with a kappa LC) HC
QVQLQQSGPELVKPGASVKISCKASGYAFSSSWLNWVKQRPGEGLEWIGRIYPGDGDINYNGKFKGKATLTADKSSSTAYMQLSSLTSDDSAVYFCTKRGRYALDYWGQGTSVTVSSAKTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGK

SEQ ID NO: 49 - protein, 218 aa, Artificial Sequence
  ACI-31-30C11-Ab1 (murine IgG2b with a kappa LC) LC
DIVLTQSPASLAVSLGQRATISCRASKSVDNYGKSFMHWFQQKPGQPPKLLIYRASNLESGIPARFSGSGSRTDFTLTINPVEADDVATYYCQQSNEDPYTFGGGTKLEMKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 50 - protein, 447 aa, Artificial Sequence
  ACI-31-30C11-Ab1 (murine IgG2a with a kappa LC) HC
QVQLQQSGPELVKPGASVKISCKASGYAFSSSWLNWVKQRPGEGLEWIGRIYPGDGDINYNGKFKGKATLTADKSSSTAYMQLSSLTSDDSAVYFCTKRGRYALDYWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 51 - protein, 218 aa, Artificial Sequence; identical to SEQ ID NO: 49
  ACI-31-30C11-Ab1 (murine IgG2a with a kappa LC) LC

SEQ ID NO: 52 - DNA, 366 bp, Artificial Sequence
  ACI-31-25B1-Ab2 (IgG2b and IgG2a) HCVR
gaggtgcagctggtggagtctgggggagacttagtgaagcctggagggtccctgaaactctcctgtgcagcctctagattcactttcagtacctatggcatgtcttgggttcgccagactccagacaagaggctggaatgggtcgcaaccattagtagtggtagaagttacacctactatgcagacagtgtgaaggggcgattcaccatctccagagacaatgccaagaacaccctgtacctgcaaatgagcagtctgaagtctgaggacacagccatgtattactgtgcaagaaatagtaacaaccagagggattactatgctatggactactggggtcaaggaacctcagtcaccgtctcctca

SEQ ID NO: 53 - DNA, 345 bp, Artificial Sequence
  ACI-31-25B1-Ab2 (IgG2b and IgG2a) LCVR
caacttgtgctcactcagtcatcttcagcctctttctccctgggagcctcagcaaaactcacgtgcaccttgagtagtcagcacattacgtacaccattgagtggtatcagcaacagccactcaagcctcctaagtatgtgatggtacttgagaaagatggaagccacagcacaggtgatgggattcctgatcgcttctctggatccagctctggtgctgatcgttaccttagcatttccaacatccagcctgaagatgaagcagtatacatctgtggtgtgggtgatgcaattaaggaacaatttgtgtatgttttcggcggtggaaccaaggtcactgtccta

SEQ ID NO: 54 - DNA, 351 bp, Artificial Sequence
  ACI-31-30C11-Ab1 (IgG2b and IgG2a) HCVR
caggttcagctgcagcagtctggacctgagctggtgaagcctggggcctcagtgaagatttcctgcaaggcttctggctacgcattcagtagctcctggctgaactgggtgaagcagaggcctggagagggtcttgagtggattggacggatttatcctggagatggagatattaactacaatgggaagttcaagggcaaggccacactgactgcagacaaatcctccagcacagcctacatgcaactcagcagcctgacatctgacgactctgcggtctacttctgtacaaagcggggacgctatgctttggactactggggtcaaggaacctcagtcaccgtctcctca

SEQ ID NO: 55 - DNA, 333 bp, Artificial Sequence
  ACI-31-30C11-Ab1 (IgG2b and IgG2a) LCVR
gacattgtgctgacccaatctccagcttctttggctgtgtctctagggcagagggccaccatatcctgcagagccagtaaaagtgttgataattatggcaaaagttttatgcactggttccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatccgtacacgttcggaggggggaccaagctggaaatgaaa

SEQ ID NO: 56 - protein, 330 aa, Artificial Sequence
  Murine IgG2a HC constant domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 57 - protein, 107 aa, Artificial Sequence
  Murine LC kappa domain
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 58 - protein, 452 aa, Artificial Sequence
  ACI-31-25B1-Ab2 human IgG1 HC
EVQLVESGGDLVKPGGSLKLSCAASRFTFSTYGMSWVRQTPDKRLEWVATISSGRSYTYYADSVKGRFTISRDNAKNTLYLQMSSLKSEDTAMYYCARNSNNQRDYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 221 aa, Artificial Sequence
  ACI-31-25B1-Ab2 human IgG1/ Lambda LC
QLVLTQSSSASFSLGASAKLTCTLSSQHITYTIEWYQQQPLKPPKYVMVLEKDGSHSTGDGIPDRFSGSSSGADRYLSISNIQPEDEAVYICGVGDAIKEQFVYVFGGGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 60 - protein, 121 aa, Artificial Sequence
  ACI-8037-103H5-Ab2 HCVR
QVTLKESGPGILKPSQTLSLTCSFSGFSLSTSGMGVGWIRQPSGKGLEWLAHIWWDDDKYYNPSLKSRLTISKDSSRNQVFLKITSVDTADTATYYCVRRGTRLEDYFDFWGQGTTLTVSS

SEQ ID NO: 61 - protein, 7 aa, Artificial Sequence
  ACI-8037-103H5-Ab2 HC CDR1
TSGMGVG

SEQ ID NO: 62 - protein, 16 aa, Artificial Sequence
  ACI-8037-103H5-Ab2 HC CDR2
HIWWDDDKYYNPSLKS

SEQ ID NO: 63 - protein, 11 aa, Artificial Sequence
  ACI-8037-103H5-Ab2 HC CDR3
RGTRLEDYFDF

SEQ ID NO: 64 - protein, 112 aa, Artificial Sequence
  ACI-8037-103H5-Ab2 LCVR
DVLMTQTPLSLPVSLGDQASISCRSSRSIVHSNGNTYLEWYLQKPGQSPKLLIFKVSNRFSGVPDRFSGSGSGTDFTLKISRVEAEDQGVYYCFQGSHVPLTFGAGTKLELK

SEQ ID NO: 65 - protein, 16 aa, Artificial Sequence
  ACI-8037-103H5-Ab2 LC CDR1
RSSRSIVHSNGNTYLE

SEQ ID NO: 66-67 - skipped (2 entries)

SEQ ID NO: 68 - DNA, 363 bp, Artificial Sequence
  ACI-8037-103H5-Ab2 HCVR
caagttactctaaaagagtctggccctgggatattgaagccctcacagaccctcagtctgacttgttctttctctgggttttcactgagcacttctggtatgggtgttggctggattcgtcagccttccgggaagggtctggagtggctggcacacatttggtgggatgatgataagtactataacccatccctgaagagccggctcacaatctccaaggattcctccagaaaccaggtattcctcaagatcaccagtgtggacactgcagatactgccacttactactgtgttcgaagggggactaggttagaggactactttgacttctggggccaaggcaccactctcacagtctcctca

SEQ ID NO: 69 - DNA, 336 bp, Artificial Sequence
  ACI-8037-103H5-Ab2 LCVR
gatgttttgatgacccaaactccactctccctgcctgtcagtcttggggatcaagcctccatctcttgcagatctagtcggagcattgttcatagtaatggaaacacctatttagaatggtacctgcagaaaccaggccagtctccaaagctcctgatcttcaaagtttccaaccgattttctggggtcccagacaggttcagtggcagtggatcagggacagatttcacactcaagatcagcagagtggaggctgaggatcagggagtttattactgctttcaaggttcacatgttccgctcacgttcggtgctgggaccaagctggagctgaaa

SEQ ID NO: 70 - protein, 122 aa, Artificial Sequence
  ACI-8037-109F4-Ab1 HCVR
QVTLKESGPGILQPSQTLSLTCSFSGFSLSTSGMGVSWIRQPSGKGLEWLAHIYWDDDKRYNPSLKSRLTISKDTSRNQVFLKITSVDTADAGTYYCARRPIRTVVDAMDYWGQGTSVTVSL

SEQ ID NO: 71-72 - skipped (2 entries)

SEQ ID NO: 73 - protein, 12 aa, Artificial Sequence
  ACI-8037-109F4-Ab1 HC CDR3
RPIRTVVDAMDY

SEQ ID NO: 74 - protein, 112 aa, Artificial Sequence
  ACI-8037-109F4-Ab1 LCVR
DVLMTQTPLSLPVSLGDQASISCRSSQTIVHSNGNTYLEWYLQKPGQSPNLLIYKVSNRFSGVPDRFSGSGSGTDFTLKISRVEAEDLGVYYCFQGSHVPLTFGAGTKLELK

SEQ ID NO: 75 - protein, 16 aa, Artificial Sequence
  ACI-8037-109F4-Ab1 VL CDR1
RSSQTIVHSNGNTYLE

SEQ ID NO: 76-77 - skipped (2 entries)

SEQ ID NO: 78 - DNA, 366 bp, Artificial Sequence
  ACI-8037-109F4-Ab1 HCVR
caggttactctgaaagagtctggccctgggatattgcagccctcccagaccctcagtctgacttgttctttctctgggttttcactgagcacttctggtatgggtgtgagctggattcgtcagccttcaggaaagggtctggagtggctggcacacatttactgggatgatgacaagcgctataacccatccctgaagagccggctcacaatctccaaggatacctccagaaaccaggttttcctcaagatcaccagtgtggacactgcagatgctggcacatactactgtgctcgaagacctattagaacggtagtagatgctatggactactggggtcaaggaacctcagtcaccgtctcctta

SEQ ID NO: 79 - DNA, 336 bp, Artificial Sequence
  ACI-8037-109F4-Ab1 LCVR
gatgttttgatgacccaaactccactctccctgcctgtcagtcttggagatcaggcctccatctcttgcagatctagtcagaccattgtacatagtaatggaaacacctatttggaatggtacctgcagaaaccaggccagtctccaaacctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 80 - protein, 116 aa, Artificial Sequence
  ACI-8039-306H5-Ab1 HCVR
QVTLKESGPGILKPSQTLSLTCSFSGFSLSTSGMGVGWIRQPSGKGLEWLAHIWWDDDKYYNPSLKSQLTISKDTSRNQIFLKITSVDTADTATYYCVRSLYFDYWGQGTTLTVSS

SEQ ID NO: 81-82 - skipped (2 entries)

SEQ ID NO: 83 - protein, 6 aa, Artificial Sequence
  ACI-8039-306H5-Ab1 HC CDR3
SLYFDY

SEQ ID NO: 84 - protein, 112 aa, Artificial Sequence
  ACI-8039-306H5-Ab1 HCVR
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGAGTKLELK

SEQ ID NO: 85 - protein, 16 aa, Artificial Sequence
  ACI-8039-306H5-Ab1 LC CDR1
RSSQSLVHSNGNTYLH

SEQ ID NO: 86 - skipped

SEQ ID NO: 87 - protein, 9 aa, Artificial Sequence
  ACI-8039-306H5-Ab1 LC CDR3
SQSTHVPLT

SEQ ID NO: 88 - DNA, 348 bp, Artificial Sequence
  ACI-8039-306H5-Ab1 HCVR
caagttactctaaaagagtctggccctgggatattgaagccctcacagaccctcagtctgacttgttctttctctgggttttcactgagcacttctggtatgggtgtaggctggattcgtcagccttcagggaagggtctggagtggctggcacacatttggtgggatgatgataagtactataacccatccctgaagagtcagctcacaatctccaaggatacctccagaaaccagatattcctcaagatcaccagtgtggacactgcagatactgccacttactactgtgttcgaagcctgtactttgactactggggccaaggcaccactctcacagtctcctca

SEQ ID NO: 89 - DNA, 336 bp, Artificial Sequence
  ACI-8039-306H5-Ab1 LCVR
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccgctcacgttcggtgctgggaccaagctggagctgaaa

SEQ ID NO: 90 - protein, 117 aa, Artificial Sequence
  ACI-8039-307F7-Ab2 HCVR
QVQLQQPGAELVRPGGSVKLSCKASGYSFTNYWMNWVKQRPGQGLEWIGMIHPSDSETRLNQKFKDKATLTVDKSSSTAYLQLSSPTSEDSAVYYCAKLLLYYFDYWGQGTTLTVSS

SEQ ID NO: 91 - protein, 5 aa, Artificial Sequence
  ACI-8039-307F7-Ab2 HC CDR1
NYWMN

SEQ ID NO: 92 - protein, 17 aa, Artificial Sequence
  ACI-8039-307F7-Ab2 HC CDR2
MIHPSDSETRLNQKFKD

SEQ ID NO: 93 - protein, 8 aa, Artificial Sequence
  ACI-8039-307F7-Ab2 HC CDR3
LLLYYFDY

SEQ ID NO: 94 - protein, 113 aa, Artificial Sequence
  ACI-8039-307F7-Ab2 LCVR
DIVMTQSPSSLAMSVGQKVTMSCKSSQSLLNSSNQKNYLAWYQQKPGQSPKILVYFASTRESGVPDRFRGSGSGTDFTLTISSVQAEDLADYFCQQHYSPPPTFGGGTKLEIK

SEQ ID NO: 95 - protein, 17 aa, Artificial Sequence
  ACI-8039-307F7-Ab2 LC CDR1
KSSQSLLNSSNQKNYLA

SEQ ID NO: 96 - protein, 7 aa, Artificial Sequence
  ACI-8039-307F7-Ab2 LC CDR2
FASTRES

SEQ ID NO: 97 - protein, 9 aa, Artificial Sequence
  ACI-8039-307F7-Ab2 LC CDR3
QQHYSPPPT

SEQ ID NO: 98 - DNA, 351 bp, Artificial Sequence
  ACI-8039-307F7-Ab2 HCVR
caggtccaactgcagcagcctggggctgagctggtgaggcctggaggttcagtgaagctgtcctgcaaggcttctggctactccttcaccaactactggatgaactgggtgaagcagaggcctggacaaggccttgagtggattggcatgattcatccttccgatagtgaaactaggttaaatcagaaattcaaggacaaggccacattgactgtagacaaatcatccagcacagcctacttgcaactcagcagcccgacatctgaggactctgcggtctattactgtgcaaaattactactatactactttgactactggggccaaggcaccactctcacagtctcctca

SEQ ID NO: 99 - DNA, 339 bp, Artificial Sequence
  ACI-8039-307F7-Ab2 LCVR
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaaattctggtatactttgcatccactagggaatctggggtccctgatcgcttcagaggcagtggatctgggacagatttcactctaaccatcagcagtgtgcaggctgaagacctggcagattacttctgtcagcaacattatagccctcctccgacgttcggtggaggcaccaagctggaaatcaaa

SEQ ID NO: 100 - protein, 116 aa, Artificial Sequence
  ACI-8039-309D9-Ab2 HCVR
QVQLQQSGPQLVRPGASVKISCKASGNSFTSYWMHWVKQRPGQGLEWIGMIDPSDSETRLNQKFKDKATLTVDKSSSTAYMQLSSPTSEDSAVYYCAGLYFTLDYWGQGTSVTVSS

SEQ ID NO: 101 - protein, 5 aa, Artificial Sequence
  ACI-8039-309D9-Ab2 HC CDR1
SYWMH

SEQ ID NO: 102 - protein, 17 aa, Artificial Sequence
  ACI-8039-309D9-Ab2 HC CDR2
MIDPSDSETRLNQKFKD

SEQ ID NO: 103 - protein, 7 aa, Artificial Sequence
  ACI-8039-309D9-Ab2 HC CDR3
LYFTLDY

SEQ ID NO: 104 - protein, 113 aa, Artificial Sequence
  ACI-8039-309D9-Ab2 LCVR
DILMTQSPSSLTVSAGEKVTMSCKSSQSLLASGNQNNYLAWHQQKPGRSPKILIIWASTRVSGVPDRFIGSGSGTDFTLTINSVQAEDLAVYYCQQSYSAPLTFGAGTKLELK

SEQ ID NO: 105 - protein, 17 aa, Artificial Sequence
  ACI-8039-309D9-Ab2 LC CDR1
KSSQSLLASGNQNNYLA

SEQ ID NO: 106 - protein, 7 aa, Artificial Sequence
  ACI-8039-309D9-Ab2 LC CDR2
WASTRVS

SEQ ID NO: 107 - protein, 9 aa, Artificial Sequence
  ACI-8039-309D9-Ab2 LC CDR3
QQSYSAPLT

SEQ ID NO: 108 - DNA, 348 bp, Artificial Sequence
  ACI-8039-309D9-Ab2 HCVR
caggtgcaactgcagcagtctgggcctcagctggttagacctggggcttcagtgaagatatcctgcaaggcttctggtaactcattcaccagctactggatgcactgggtgaagcagaggcctggacaaggtcttgagtggattggcatgattgatccttccgatagtgagactaggttaaatcagaagttcaaggacaaggccacattgactgtagacaaatcctccagcacagcctacatgcaactcagcagcccgacatctgaggactctgcggtctattactgtgcggggctgtactttactttggactactggggtcaaggaacctcagtcaccgtctcctca

SEQ ID NO: 109 - DNA, 339 bp, Artificial Sequence
  ACI-8039-309D9-Ab2 LCVR
gacattttgatgacccagtctccatcctccctgactgtgtcagcaggagagaaggtcactatgagctgcaagtccagtcagagtcttttagctagtggcaaccaaaataactacttggcctggcaccagcagaaaccaggacgatctcctaaaatactgataatttgggcatccactagggtatctggagtccctgatcgcttcataggcagtgggtctgggacggatttcactctgaccatcaacagtgtgcaggctgaagatctggctgtttattactgtcagcagtcctacagcgctccgctcacgttcggtgctgggaccaagctggagctgaaa

SEQ ID NO: 110 - protein, 112 aa, Artificial Sequence
  ACI-8041-1F11B5-Ab1 HCVR
QVQLKESGPGLVAPSQSLSITCTVSGFSLTNYGVSWIRQPPGKGLEWLGVIWGDGSTNYHSALISRLSISKDNSKSQVFLKLNSLQTDDTATYYCTKGAGSWGQGIPVTVSA

SEQ ID NO: 111 - protein, 5 aa, Artificial Sequence
  ACI-8041-1F11B5-Ab1 HC CDR1
NYGVS

SEQ ID NO: 112 - protein, 16 aa, Artificial Sequence
  ACI-8041-1F11B5-Ab1 HC CDR2
VIWGDGSTNYHSALIS

SEQ ID NO: 113 - protein, 4 aa, Artificial Sequence
  ACI-8041-1F11B5-Ab1 HC CDR3
GAGS

SEQ ID NO: 114 - protein, 112 aa, Artificial Sequence
  ACI-8041-1F11B5-Ab1 LCVR
DVVLTQTPLTLSVTIGQPASISCKSSQSLLDSDGRTYLIWLLQRPGQSPKRLIYLVSTLDSGVPDRFTGSGSGTDFTLKISRVEAEDLGVYYCWQGTHFPWTFGGGTKLEIK

SEQ ID NO: 115 - protein, 16 aa, Artificial Sequence
  ACI-8041-1F11B5-Ab1 LC CDR1
KSSQSLLDSDGRTYLI

SEQ ID NO: 116 - protein, 7 aa, Artificial Sequence
  ACI-8041-1F11B5-Ab1 LC CDR2
LVSTLDS

SEQ ID NO: 117 - protein, 9 aa, Artificial Sequence
  ACI-8041-1F11B5-Ab1 LC CDR3
WQGTHFPWT

SEQ ID NO: 118 - DNA, 336 bp, Artificial Sequence
  ACI-8041-1F11B5-Ab1 HCVR
caggtgcagctgaaggagtcaggacctggcctggtggcgccctcacagagcctgtccatcacatgcactgtctcagggttctcattaaccaactatggtgtaagctggattcgccagcctccaggaaagggtctggagtggctgggagtaatatggggtgacgggagcacaaattatcattcagctctcatatccagactgagcatcagcaaggataactccaagagccaagttttcttaaaactgaacagtctgcaaactgatgacacagccacgtactactgtaccaaaggagctggctcttggggccaagggattccggtcactgtctctgca

SEQ ID NO: 119 - DNA, 336 bp, Artificial Sequence
  ACI-8041-1F11B5-Ab1 LCVR
gatgttgtgctgacccagactccactcactttgtcggttaccattggacaaccagcctccatctcttgcaagtcaagtcagagcctcttagatagtgatggaaggacatatttaatttggttgttacagaggccaggccagtctccaaagcgcctaatctatctggtgtcaacactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 120 - protein, 113 aa, Artificial Sequence
  ACI-8041-2B9H5-Ab1 HCVR
QVQLQQSDAELVKPGASVKISCKVSGYTFTDHPIHWMKQRPDQGLEWIGYFYPRDSSTHYNEKFKGKATLTADKSSSTAYMQLNSLTSEDSAVYFCARSMGYWGQGTSVTVSS

SEQ ID NO: 121 - protein, 5 aa, Artificial Sequence
  ACI-8041-2B9H5-Ab1 HC CDR1
DHPIH

SEQ ID NO: 122 - protein, 17 aa, Artificial Sequence
  ACI-8041-2B9H5-Ab1 HC CDR2
YFYPRDSSTHYNEKFKG

SEQ ID NO: 123 - protein, 4 aa, Artificial Sequence
  ACI-8041-2B9H5-Ab1 HC CDR3
SMGY

SEQ ID NO: 124 - protein, 107 aa, Artificial Sequence
  ACI-8041-2B9H5-Ab1 LCVR
DIKMTQSPSSMYASLGERVTITCKASQDINSYLSWFQQKPGKSPKTLIYRANRLVDGVPSRFSGSGSGQDYSLTISSLDYEDMGIYYCLQYDEFPPTFGAGTKLELK

SEQ ID NO: 125 - protein, 11 aa, Artificial Sequence
  ACI-8041-2B9H5-Ab1 LC CDR1
KASQDINSYLS

SEQ ID NO: 126 - protein, 7 aa, Artificial Sequence
  ACI-8041-2B9H5-Ab1 LC CDR2
RANRLVD

SEQ ID NO: 127 - protein, 9 aa, Artificial Sequence
  ACI-8041-2B9H5-Ab1 LC CDR3
LQYDEFPPT

SEQ ID NO: 128 - DNA, 339 bp, Artificial Sequence
  ACI-8041-2B9H5-Ab1 HCVR
caggttcagctgcaacagtctgacgctgagttggtgaaacctggagcttcagtgaagatatcctgcaaggtttctggctacaccttcactgaccatcctattcactggatgaagcagaggcctgaccagggcctggaatggattggatatttttatcctagagatagtagtactcactacaatgagaagttcaagggcaaggccacattgactgcagacaaatcctccagcacagcctacatgcagctcaacagcctgacatctgaggactctgcagtctacttctgtgcaagatcgatgggctactggggtcaaggaacctcagtcaccgtctcctca

SEQ ID NO: 129 - DNA, 321 bp, Artificial Sequence
  ACI-8041-2B9H5-Ab1 LCVR
gacatcaagatgacccagtctccatcttccatgtatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagtctggactatgaagatatgggaatttattattgtctacagtatgatgagtttcctcccacgttcggtgctgggaccaagctggagctgaaa

SEQ ID NO: 130 - protein, 113 aa, Artificial Sequence
  ACI-8041-3G11B3-Ab1 HCVR
QVQLQQSGAELVKPGASVKLSCKASGYTFINYYIYWVKQRPGQGLEWIGEIIPSNDGTNFNEKFKTKATLTVDKSSSTAYMQLSSLTSEDSAVYYCTATGTVWGQGTLVTVSA

SEQ ID NO: 131 - protein, 5 aa, Artificial Sequence
  ACI-8041-3G11B3-Ab1 HC CDR1
NYYIY

SEQ ID NO: 132 - protein, 17 aa, Artificial Sequence
  ACI-8041-3G11B3-Ab1 HC CDR2
EIIPSNDGTNFNEKFKT

SEQ ID NO: 133 - protein, 4 aa, Artificial Sequence
  ACI-8041-3G11B3-Ab1 HC CDR3
TGTV

SEQ ID NO: 134 - protein, 112 aa, Artificial Sequence
  ACI-8041-3G11B3-Ab1 LCVR
DVLMTQTPLSLPVSLGDQASISCRSSQSIVHSNGITYLEWYLQKPGQSPKLLIYKVSNRFSGVPDRFSGSGSGTDFTLKISRVEAEDLGVYYCFQGSHVPWTFGGGTKLEIK

SEQ ID NO: 135 - protein, 16 aa, Artificial Sequence
  ACI-8041-3G11B3-Ab1 LC CDR1
RSSQSIVHSNGITYLE

SEQ ID NO: 136 - skipped

SEQ ID NO: 137 - protein, 9 aa, Artificial Sequence
  ACI-8041-3G11B3-Ab1 LC CDR3
FQGSHVPWT

SEQ ID NO: 138 - DNA, 339 bp, Artificial Sequence
  ACI-8041-3G11B3-Ab1 HCVR
caggtccaactgcagcagtctggggctgaactggtgaagcctggggcttcagtgaagttgtcctgcaaggcttctggctacaccttcatcaactactatatatactgggtgaaacagaggcctggacaaggccttgagtggattggagagattattcctagcaatgatggtactaacttcaatgagaagttcaagaccaaggccacactgactgtagacaaatcctccagcacagcatacatgcaactcagcagcctgacatctgaggactctgcggtctattactgtaccgcgacagggacggtctggggccaagggactctggtcactgtctctgca

SEQ ID NO: 139 - DNA, 336 bp, Artificial Sequence
  ACI-8041-3G11B3-Ab1 LCVR
gatgttttgatgacccaaactccactctccctgcctgtcagtcttggagatcaagcctccatctcttgcagatctagtcagagcattgtacatagtaatggaatcacctatttagaatggtacctgcagaaaccaggccagtctccaaagctcctaatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 140 - protein, 113 aa, Artificial Sequence
  ACI-8041-4G6D2-Ab1 HCVR
QVQLQQSGAELVRPGASVKLSCKASGYTFTSYYMYWVKQRPGQGLEWIGEIIPSNGDTNFNEKFRSKATLTVDKSSSTAYMQLSSLTSEDSAVYYCTATGTVWGQGTLVTVSA

SEQ ID NO: 141 - protein, 5 aa, Artificial Sequence
  ACI-8041-4G6D2-Ab1 HC CDR1
SYYMY

SEQ ID NO: 142 - protein, 17 aa, Artificial Sequence
  ACI-8041-4G6D2-Ab1 HC CDR2
EIIPSNGDTNFNEKFRS

SEQ ID NO: 143 - skipped

SEQ ID NO: 144 - protein, 112 aa, Artificial Sequence
  ACI-8041-4G6D2-Ab1 LCVR
DVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPWTFGGGTKLEIK

SEQ ID NO: 145-147 - skipped (3 entries)

SEQ ID NO: 148 - DNA, 339 bp, Artificial Sequence
  ACI-8041-4G6D2-Ab1 HCVR
caggtccaactgcagcagtctggggctgaactggtgaggcctggggcttcagtgaagttgtcctgcaaggcttctggctacaccttcaccagctattatatgtactgggtgaagcagaggcctggacaaggccttgagtggattggagagattattcctagtaatggtgatactaacttcaatgagaagttcaggagcaaggccacactgactgtagacaaatcctccagcacagcatatatgcaactcagcagcctgacatctgaggactctgcggtctattactgtaccgcgacagggacggtctggggccaagggactctggtcactgtctctgca

SEQ ID NO: 149 - DNA, 336 bp, Artificial Sequence
  ACI-8041-4G6D2-Ab1 LCVR
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 150 - protein, 121 aa, Artificial Sequence
  ACI-8041-5A2D4-Ab1 HCVR
EVQLQQSGPELVKPGASVKISCKTSGYTFTENTMHWVKQIHGKSLEWIGGINPNNGVTNNNQKFEGKATLTVDKSSSTAYMELRSLTSEDSAVYYCARSDYDALYYSMDCWGQGTSVTVSS

SEQ ID NO: 151 - protein, 5 aa, Artificial Sequence
  ACI-8041-5A2D4-Ab1 HC CDR1
ENTMH

SEQ ID NO: 152 - protein, 17 aa, Artificial Sequence
  ACI-8041-5A2D4-Ab1 HC CDR2
GINPNNGVTNNNQKFEG

SEQ ID NO: 153 - protein, 12 aa, Artificial Sequence
  ACI-8041-5A2D4-Ab1 HC CDR3
SDYDALYYSMDC

SEQ ID NO: 154 - protein, 108 aa, Artificial Sequence
  ACI-8041-5A2D4-Ab1 LCVR
EIVLTQSPTTMAASPGEKITITCSGSSSIISNYLHWYQQKPGFSPKLLIYRTSNLASGVPTRFSGSGSGTSYSLTIGTMEAEDVATYYCQQGSSIPFTFGSGTKLEIK

SEQ ID NO: 155 - protein, 12 aa, Artificial Sequence
  ACI-8041-5A2D4-Ab1 LC CDR1
SGSSSIISNYLH

SEQ ID NO: 156 - protein, 7 aa, Artificial Sequence
  ACI-8041-5A2D4-Ab1 LC CDR2
RTSNLAS

SEQ ID NO: 157 - protein, 9 aa, Artificial Sequence
  ACI-8041-5A2D4-Ab1 LC CDR3
QQGSSIPFT

SEQ ID NO: 158 - DNA, 363 bp, Artificial Sequence
  ACI-8041-5A2D4-Ab1 HCVR
gaggtccagctgcaacagtctggacctgagctggtgaagcctggggcttcagtgaagatatcctgcaagacttctggatacacattcactgaaaacaccatgcactgggtgaagcagatccatggaaagagccttgagtggattggaggtattaatcctaacaatggtgttactaacaacaaccagaagttcgagggcaaggccactttgactgtagacaagtcctccagcacagcctacatggagctccgcagcctgacatctgaggattctgcagtctattactgtgcaagatctgattacgacgccctttactattctatggactgctggggtcaaggaacctcagtcaccgtctcctca

SEQ ID NO: 159 - DNA, 324 bp, Artificial Sequence
  ACI-8041-5A2D4-Ab1 LCVR
gaaattgtgctcacccagtctccaaccaccatggctgcatctcccggggagaagatcactatcacctgcagtggtagctcaagtataatttccaattacttgcattggtatcagcagaagccaggattctcccctaaactcttgatttataggacatccaatctggcttctggagtcccaactcgcttcagtggcagtgggtctgggacctcttactctctcacaattggcaccatggaggctgaagatgttgccacttactactgccagcagggtagtagtataccattcacgttcggctcggggacaaagttggaaataaaa

SEQ ID NO: 160 - protein, 124 aa, Artificial Sequence
  ACI-8041-5A6H9-Ab1 HCVR
DVQLQESGPGLVKPSQSLSLTCTVTGYSITSDYAWNWIRQFPGNKLEWMGYIDYSGITSYNPSLKSRISITRDTSKNQFFLQLNSVTTEDSATYYCARISGLDYDVRAGWFASWGQGTLVTVSA

SEQ ID NO: 161 - protein, 6 aa, Artificial Sequence
  ACI-8041-5A6H9-Ab1 HC CDR1
SDYAWN

SEQ ID NO: 162 - protein, 16 aa, Artificial Sequence
  ACI-8041-5A6H9-Ab1 HC CDR2
YIDYSGITSYNPSLKS

SEQ ID NO: 163 - protein, 15 aa, Artificial Sequence
  ACI-8041-5A6H9-Ab1 HC CDR3
ISGLDYDVRAGWFAS

SEQ ID NO: 164 - protein, 112 aa, Artificial Sequence
  ACI-8041-5A6H9-Ab1 LCVR
DIVMTQAAFSNPVTLGTSVSISCRSSKSLLHSNGITYLYWYLQKPGQSPQLLIYQMSNLASGVPDRFSSSGSGTDFTLRISRVEAEDVGVYYCAQNLERPLTFGAGTKLELK

SEQ ID NO: 165 - protein, 16 aa, Artificial Sequence
  ACI-8041-5A6H9-Ab1 LC CDR1
RSSKSLLHSNGITYLY

SEQ ID NO: 166 - protein, 7 aa, Artificial Sequence
  ACI-8041-5A6H9-Ab1 LC CDR2
QMSNLAS

SEQ ID NO: 167 - protein, 9 aa, Artificial Sequence
  ACI-8041-5A6H9-Ab1 LC CDR3
AQNLERPLT

SEQ ID NO: 168 - DNA, 372 bp, Artificial Sequence
  ACI-8041-5A6H9-Ab1 HCVR
gatgtgcagcttcaggagtcgggacctggcctggtgaaaccttctcagtctctgtccctcacctgcactgtcactggctactcaatcaccagtgattatgcctggaactggatccggcagtttccaggaaacaaactggagtggatgggctacatagactacagtggtatcactagctacaacccatctctcaaaagtcgaatctctatcactcgagacacatccaagaaccagttcttcctgcagttgaattctgtgactactgaggactcagccacatattactgtgcaagaatctctggactggattacgacgtgagggctggctggtttgcttcctggggccaagggactctggtcactgtctctgca

SEQ ID NO: 169 - DNA, 336 bp, Artificial Sequence
  ACI-8041-5A6H9-Ab1 LCVR
gatattgtgatgacgcaggctgcattctccaatccagtcactcttggaacatcagtttccatctcctgcaggtctagtaagagtctcctacatagtaatggcatcacttatttgtattggtatctgcagaagccaggccagtctcctcagctcctgatttatcagatgtccaaccttgcctcaggagtcccagacaggttcagtagcagtgggtcaggaactgatttcacactgagaatcagcagagtggaggctgaggatgtgggtgtttattactgtgctcaaaatctagaacgtccgctcacgttcggtgctgggaccaagctggagctgaaa

SEQ ID NO: 170 - protein, 121 aa, Artificial Sequence
  ACI-8041-6C3C4-Ab1 HCVR
EVQLQQSGPELVKPGASVNISCKTSGYTFTEYTMHWMKQSHGKSLEWIGGINPNNGVTGYNQKFKGRATLTVDKSSSTAYMELRSLTSEDSAVYYCARSDYDDLYYAMDYWGQGTSVTVSS

SEQ ID NO: 171 - protein, 5 aa, Artificial Sequence
  ACI-8041-6C3C4-Ab1 HC CDR1
EYTMH

SEQ ID NO: 172 - protein, 17 aa, Artificial Sequence
  ACI-8041-6C3C4-Ab1 HC CDR2
GINPNNGVTGYNQKFKG

SEQ ID NO: 173 - protein, 12 aa, Artificial Sequence
  ACI-8041-6C3C4-Ab1 HC CDR3
SDYDDLYYAMDY

SEQ ID NO: 174 - protein, 108 aa, Artificial Sequence
  ACI-8041-6C3C4-Ab1 LCVR
EIVLTQSPTTMAASPGEKITITCSASSSISSNYLHWYQQKPGFSPKLLIYRTSNLASGVPARFSGSGSGTSYSLTIGTMEAEDVATYYCQQGSSIPFTFGSGTKLEIK

SEQ ID NO: 175 - protein, 12 aa, Artificial Sequence
  ACI-8041-6C3C4-Ab1 LC CDR1
SASSSISSNYLH

SEQ ID NO: 176-177 - skipped (2 entries)

SEQ ID NO: 178 - DNA, 363 bp, Artificial Sequence
  ACI-8041-6C3C4-Ab1 HCVR
gaggtccagctgcaacagtctggacctgagctggtgaagcctggggcttcagtgaacatatcctgcaagacttctggatacacattcactgaatacaccatgcactggatgaagcagagccatggaaagagccttgagtggattggaggtattaatcctaacaatggtgttactggctacaaccagaagttcaagggcagggccacattgactgtagacaagtcctccagcacagcctacatggagctccgcagcctgacatctgaggattctgcagtctattactgtgcaagatctgattacgacgacctttactatgctatggactactggggtcaaggaacctcagtcaccgtctcctca

SEQ ID NO: 179 - DNA, 324 bp, Artificial Sequence
  ACI-8041-6C3C4-Ab1 LCVR
gaaattgtgctcacccagtctccaaccaccatggctgcatctcccggggagaagatcactatcacctgcagtgccagctcaagtataagttccaattacttgcattggtatcagcagaagccaggattctcccctaaactcttgatttataggacatccaatctggcttctggagtcccagctcgcttcagtggcagtgggtctgggacctcttactctctcacaattggcaccatggaggctgaagatgttgccacttactactgccagcagggtagtagtataccattcacgttcggctcggggacaaagttggaaataaaa

SEQ ID NO: 180 - protein, 120 aa, Artificial Sequence
  ACI-8041-9D7E1-Ab1 HCVR
EVMLVESGGGLVKPGGSLKLSCAASGFTFSSYAMSWVRQTPEKRLEWVATISSGGNYTYCPDSVKGRFTISRDNAKNTLYLQMSSLRSEDTAMYYCAAYAYDDGYYFDYWGQGTTLTVSS

SEQ ID NO: 181 - protein, 5 aa, Artificial Sequence
  ACI-8041-9D7E1-Ab1 HC CDR1
SYAMS

SEQ ID NO: 182 - protein, 17 aa, Artificial Sequence
  ACI-8041-9D7E1-Ab1 HC CDR2
TISSGGNYTYCPDSVKG

SEQ ID NO: 183 - protein, 11 aa, Artificial Sequence
  ACI-8041-9D7E1-Ab1 HC CDR3
YAYDDGYYFDY

SEQ ID NO: 184 - protein, 113 aa, Artificial Sequence
  ACI-8041-9D7E1-Ab1 LCVR
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYSTPLTFGAGTKLELK

SEQ ID NO: 185-186 - skipped (2 entries)

SEQ ID NO: 187 - protein, 9 aa, Artificial Sequence
  ACI-8041-9D7E1-Ab1 LC CDR3
QQHYSTPLT

SEQ ID NO: 188 - DNA, 360 bp, Artificial Sequence
  ACI-8041-9D7E1-Ab1 HCVR
gaagtgatgctggtggagtctgggggaggcttagtgaagcctggagggtccctgaaactctcctgtgcagcctctggattcactttcagtagctatgccatgtcttgggttcgccagactccggagaagaggctggagtgggtcgcaaccattagtagtggtggtaattacacctactgtcccgacagtgtgaaggggcgattcaccatctccagagacaatgccaagaacaccctgtacctgcaaatgagcagtctgaggtctgaggacacggccatgtattactgtgcagcctatgcttacgacgacgggtactactttgactactggggccaaggcaccactctcacagtctcctca

SEQ ID NO: 189 - DNA, 339 bp, Artificial Sequence
  ACI-8041-9D7E1-Ab1 LCVR
gacattgtgatgacacagtctccatcctccctggctatgtcagtaggacagaaggtcactatgagctgcaagtccagtcagagccttttaaatagtagcaatcaaaagaactatttggcctggtaccagcagaaaccaggacagtctcctaaacttctggtatactttgcatccactagggaatctggggtccctgatcgcttcataggcagtggatctgggacagatttcactcttaccatcagcagtgtgcaggctgaagacctggcagattacttctgtcagcaacattatagcactcctctcacgttcggtgctgggaccaagctggagctgaaa

SEQ ID NO: 190-193 - skipped (4 entries)

SEQ ID NO: 194 - protein, 106 aa, Artificial Sequence
  ACI-8041-10H3B11-Ab1 LCVR
DIQMTQSPSSLSASLGGKVTITCKASQDINKYIAWYQHKPGKGPRLLIHYTSTLQPGIPSRFSGSGSGRDYSFSISNLGPEDIATYYCLQYDNLLTFGGGTKLEIK

SEQ ID NO: 195 - protein, 11 aa, Artificial Sequence
  ACI-8041-10H3B11-Ab1 LC CDR1
KASQDINKYIA

SEQ ID NO: 196 - protein, 7 aa, Artificial Sequence
  ACI-8041-10H3B11-Ab1 LC CDR2
YTSTLQP

SEQ ID NO: 197 - protein, 8 aa, Artificial Sequence
  ACI-8041-10H3B11-Ab1 LC CDR3
LQYDNLLT

SEQ ID NO: 198 - skipped

SEQ ID NO: 199 - DNA, 318 bp, Artificial Sequence
  ACI-8041-10H3B11-Ab1 LCVR
gacatccagatgacacagtctccatcctcactgtctgcatctctgggaggcaaagtcaccatcacttgcaaggcaagccaagacattaacaagtatatagcttggtaccaacacaagcctggaaaaggtcctaggctgctcattcattacacatctaccttacagccaggcatcccatcaaggttcagtggaagtgggtctgggagagattattccttcagcatcagcaacctggggcctgaagatattgcaacttattattgtctacagtatgataatcttctgacgttcggtggaggcaccaagctggaaatcaaa

(19) United States
(12) Patent Application Publication
(10) Pub. No.: US 2023/0034474 A1
(43) Pub. Date: Feb. 2, 2023

(54) Novel Molecules for Diagnosis

(71) Applicant: AC Immune SA, Lausanne (CH)

(72) Inventors: Oskar Mario Adolfsson, Lausanne (CH); Katarzyna Ewa Piorkowska, Lausanne (CH)

(21) Appl. No.: 17/640,656

(22) PCT Filed: Sep. 10, 2020
(86) PCT No.: PCT/EP2020/075420

§ 371 Date: Mar. 4, 2022

(30) Foreign Application Priority Data
Sep. 10, 2019 (EP) 19196512.8
Jul. 6, 2020 (EP) 20184340.6

(51) Int. Cl. C07K 14/47; C07K 16/18; G01N 33/68; A61P 25/28

(52) U.S. Cl. CPC C07K 14/4711; C07K 16/18; G01N 33/6896; A61P 25/28; C07K 2317/24; C07K 2317/565; C07K 2317/92; C07K 2317/51

(57) ABSTRACT

The present invention relates to novel amyloid-beta (abeta) binding molecules, in particular to abeta antibodies or antigen-binding fragments thereof and/or uses thereof. The provided molecules can also be used for determining a predisposition to amyloid-beta associated diseases, disorders or conditions, monitoring residual disorder of a disease or condition, or predicting the responsiveness of a patient who is suffering from such disease or condition to the treatment with a certain medicament. Thus, the invention relates to novel molecules that can be employed for the diagnosis of diseases, disorders or conditions associated with amyloid-beta. A sandwich immunoassay may be based on capture and detection amyloid-beta binding antibodies or antigen-binding fragments thereof in which one or other of the capture or detection antibody or antigen-binding fragment thereof displays no cross-reactivity to soluble amyloid precursor protein (APP). The other amyloid-beta binding antibody or antigen-binding fragment my display cross-reactivity to soluble amyloid precursor protein (APP) without compromising the specificity of the assay against soluble APP.

### INCORPORATION BY REFERENCE OF SEQUENCE LISTING PROVIDED AS A TEXT FILE

A Sequence Listing is provided herewith in a text file, BOULT-043_SEQ_LIST_ST25, created on Sep. 22, 2022 and having a size of 129,756 bytes. The contents of the text file are incorporated herein by reference in its entirety.

### FIELD OF THE INVENTION

The present invention relates to novel amyloid-beta (abeta, Abeta, Aβ) binding molecules, in particular to abeta antibodies or antigen-binding fragments thereof and/or uses thereof. The provided molecules can also be used for determining a predisposition to amyloid-beta associated diseases, disorders or conditions, monitoring the disease, disorder or condition, or predicting the responsiveness of a patient who is suffering from such disease, disorder or condition to the treatment with a certain medicament. Thus, the invention relates to novel molecules that can be employed for the diagnosis and/or treatment of diseases, disorders or conditions associated with amyloid-beta.

### BACKGROUND OF THE INVENTION

Alzheimer's disease (AD) is the most common cause of dementia in the elderly affecting an estimated 15 million people worldwide and 40% of the population above 85 years. The disease is characterized by progressive loss of memory, speech, and movement with a total incapacitation of the patient and eventually death. AD takes a big and devastating toll on those with the disease as well as their families, friends and caregivers.

Today, therapy focuses on controlling the symptoms of AD and its various stages. Currently available AD treatment options may help delay AD symptoms from becoming worse for a limited period of time. However, there is no evidence that these medications have any effect on the underlying progression of the disease. No cure is currently available for AD, thus early AD treatment is warranted. With no disease modifying treatment options currently available, the need for good biomarkers for earlier diagnosis is as crucial as ever. The single most prominent and early biochemical characteristic of AD is the formation of extracellular amyloid-beta (Aβ) plaques in the brain (Calderon-Garciduenas & Duyckaerts, 2017), primarily containing the 42-amino acid form of Aβ. Aβ plaques are formed by the 39 to 43 amino acid long Aβ peptide (resulting from the cleavage of the amyloid precursor protein (APP)), which is in random coil conformation in its natural non-pathological form. During the transition to the pathological state, it transforms mainly into a β-sheet secondary structure, spontaneously aggregating into insoluble deposits. Even if the precise role of Aβ in the development of AD is still debated, it is widely accepted that soluble Aβ oligomers impair synapse structure and function, and that the smallest synaptotoxic species are dimers or small soluble multimers, whereas neither Aβ monomers nor plaque cores significantly alter neuronal viability or synaptic plasticity (Shankar et al., 2008).

Down syndrome (DS), also known as trisomy 21, is one of the most common causes of intellectual disability, affecting 1 in 800 newborns. This condition most commonly involves triplication of chromosome 21 (Belichenko, 2016). Subjects with DS have characteristic facial features, deficits in the immune and endocrine systems, and delayed cognitive development. A key feature of adult subjects with DS is their increased risk of developing similar clinical symptoms of Alzheimer's Disease (AD), characterized by a decline in specific cognitive domains suggestive of a diagnosis of dementia. Virtually all subjects with DS older than 40 years exhibit neuropathological changes similar to AD, in the form of senile plaque formation and neurofibrillary tangles (Head, 2012). It is well accepted that the neuropathology for AD-like cognitive decline involves the β-amyloid (Aβ) peptide deposition and subsequent plaque formation, neurofibrillary tangles, vascular damage, neuro-inflammation and ultimately neuronal cell death. The gene of the amyloid protein precursor (APP), which encodes the precursor protein of Aβ, resides on chromosome 21. In subjects with DS, the entire or at least a part of chromosome 21 is present in triplicate. Consequently, this leads to three copies of the gene that encodes APP, which results in the generation of an excess of Aβ. An increased Aβ protein production has been shown to correlate with AD-like symptoms in DS subjects as well as in the general population that develops AD (Head, 2012). These findings show conclusively that lifelong overexpression of wild-type APP causes cognitive decline in subjects with DS, in a similar way to the amyloid cascade hypothesis used to describe subjects with AD. Down syndrome-related Alzheimer's Disease is characterized by the presence of brain neuropathological hallmarks of Alzheimer's Disease (including notably the accumulation of brain amyloid plaques and neurofibrillary tangles) which can lead, when the brain lesions are sufficiently developed, to the appearance of clinical symptoms like cognitive decline and functional impairment.

The amyloid-beta associated disease, disorder or condition is a neurological disorder such as Alzheimer's Disease (AD). Other examples of amyloid-beta associated diseases, disorders or conditions according to the invention include mild cognitive impairment (MCI), Down syndrome (DS), Down syndrome-related Alzheimer's Disease, cardiac amyloidosis, cerebral amyloid angiopathy (CAA), multiple sclerosis, Parkinson's disease (PD), Parkinson's Disease with Dementia (PDD), Dementia with Lewy Body, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure. Many of these conditions are characterized by, or associated with, loss of cognitive memory capacity. Conditions characterized by, or associated with, loss of cognitive memory capacity according to the invention therefore include AD, mild cognitive impairment (MCI), Down syndrome (DS), Down syndrome-related Alzheimer's Disease, cardiac amyloidosis, cerebral amyloid angiopathy (CAA), multiple sclerosis, Parkinson's disease, Parkinson's disease with Dementia (PDD), Dementia with Lewy body, ALS (amyotrophic lateral sclerosis), Myotonic dystrophy, hepatic dysfunction or failure and inclusion body myositis (IBM).

Currently, there are no clinically validated biomarkers of patient samples (e.g., blood, cerebral spinal fluid, urine, etc.) that can be used to specifically diagnose, stratify, or monitor the progression or regression of an amyloid-beta associated disease, disorder or condition in a subject. The majority of AD biomarker studies are focused on the quantitative changes in Tau and Aβ proteins and modifications of these protein levels in the cerebral spinal fluid from AD patients, with Abeta levels showing an earlier change (Buchhave et al., 2012). For the AD biomarker and diagnostic field, there is a great need for Aβ antibodies that can be engineered and optimized for a given biomarker use and a specific diagnostic technology platform.

The early diagnosis of AD and other conditions where Aβ plays a role in the pathology, and/or the Aβ level is altered in accessible body tissue as a marker of disease progress, is crucial for early intervention. The progression of biochemical alterations in AD is slow, with a relatively long time that passes before the first clinical signs of disease are visible. An even longer time passes before a reliable diagnosis of AD can be made and it can be distinguished from other forms of dementia (Knopman et al., 2001). With neurodegeneration starting 20 or more years before clinical symptoms with a reliable diagnosis is possible, symptoms of mild cognitive impairment (MCI) may be noted, suggestive of a prodromal stage of AD (Britt et al., 2011). Although MCI is a feature at the prodromal AD stage, it has highly variable natural history with fluctuation of cognitive status over time making it an unreliable predictor of progress towards AD clinical diagnosis (Gauthier et al., 2006). Alterations of biomarkers in accessible tissues serving as surrogates of disease progress are thought to occur early during the preclinical prodromal stage of AD (Robb et al., 2017). Here we describe an antibody generated for use in Aβ biomarker assays for early diagnosis and/or treatment of diseases, disorders or conditions associated with amyloid-beta.

### DESCRIPTION OF THE INVENTION

It is an object of the present invention to provide a binding molecule, in particular an amyloid-beta antibody or antigen-binding fragment thereof, that can be employed in uses related to amyloid-beta associated diseases, disorders or conditions, such as for example diagnosis. The binding molecule, in particular the amyloid-beta antibody or antigen-binding fragment thereof, of the invention may selectively bind to or capture any amyloid-beta peptides or species in solution, in particular in body fluid or buffer solution. It was surprisingly found that the amyloid-beta antibody or antigen-binding fragment thereof, of the invention may further selectively bind to or capture any amyloid-beta peptides or species in solution independently of the conformational state of the amyloid-beta peptides or species and/or no cross-reactivity to soluble amyloid precursor protein (APP), in particular with no cross-reactivity to soluble APP produced from cleavage by alpha-secretase (referred to herein as “soluble APP alpha” or “sAPPalpha”). Reference to selective binding or selective capture means that the binding molecule does not significantly bind to any other species present in solution (apart from amyloid-beta, irrespective of conformational state), such as a body fluid. This may be applicable in both an in vitro and an in vivo context. Selectivity is important for effective performance in various diagnostic applications as described herein. Thus, in preferred embodiments, the binding molecules of the invention do not significantly bind to other protein species found in plasma (which include apolipoproteins, immunoglobulins, albumin, fibrinogens etc.). That is to say, the binding molecules of the invention are specific for amyloid-beta, irrespective of conformational state, when bound or captured in a plasma sample. The extent of binding of a binding molecule, in particular an amyloid-beta antibody or an antigen-binding fragment thereof, of the invention to an unrelated, non-amyloid-beta protein may be less than about 10% of the binding of the antibody to amyloid-beta as measured, e.g., by a radioimmunoassay (RIA). When reference is made to “no cross-reactivity” to sAPPalpha this is intended to mean that there is no appreciable binding of the binding molecule to sAPPalpha. This may be measured for example by comparing the binding to sAPPalpha with binding to Abeta1-42. When normalized against Abeta1-42 binding, the level of binding to sAPPalpha may be less than 10%, preferably less than 5% or more preferably less than 3% of binding to Abeta1-42. Such comparison may be performed by ELISA, for example an indirect ELISA. A suitable assay is described in Example 3 with reference to FIG. 8A. These antibodies are particularly useful in the invention, in particular as capture and/or detection antibodies in a sandwich immunoassay format. The terms “capture” and “detection” are terms of art. The capture antibody binds to the antigen and captures it, typically to a solid surface (to which the capture antibody is attached). The detection antibody also binds the antigen, typically to a non-overlapping epitope as the capture and detection antibodies bind at the same time. The detection antibody generates the signal for the assay. Detection may be direct (e.g. via a directly labelled antibody, such as an enzyme or fluorescent label) or indirect (e.g. using a secondary antibody which is in turn labelled). It has been found that, provided one of the capture or detection antibodies is an antibody with no cross-reactivity to sAPPalpha the sandwich immunoassay will overall be specific and give no signal for sAPPalpha, even if the other antibody does react with sAPPalpha. Specific antibodies showing no cross-reactivity to sAPPalpha are shown in Table 1 (last column, “-” indicates no binding). In some aspects of the invention an antibody with no cross-reactivity to sAPPalpha is used as both capture and detection antibody. Such an assay provides sensitive detection of abeta without detecting sAPPalpha (i.e. good specificity as well).

Thus, some binding molecules of the invention may show low cross-reactivity with sAPPalpha. They are usefully employed in combination with a binding molecule of the invention showing no cross-reactivity. Again, low cross-reactivity may be measured for example by comparing the binding to sAPPalpha with binding to Abeta1-42. When normalized against Abeta1-42 binding, the level of binding to sAPPalpha may be between greater than 10 and 60% of binding to Abeta1-42, such as around 50%. Such comparison may be performed by ELISA, for example an indirect ELISA. A suitable assay is described in Example 3 with reference to FIG. 8A. Results are shown in Table 1 for the tested antibodies of the invention. It was determined that binding molecules with low levels of cross-reactivity to sAPPalpha may be particularly advantageous when used in combination with binding molecules with no cross-reactivity to sAPPalpha. Thus, the combination of a binding molecule with low levels of cross-reactivity to sAPPalpha and a binding molecule with no cross-reactivity to sAPPalpha may be utilized according to the invention for performing sandwich immunoassays. In particular, the binding molecule with low levels of cross-reactivity to sAPPalpha may be used as capture or detection antibody and the binding molecule with no cross-reactivity to sAPPalpha may be used as detection or capture antibody. A suitable assay of the invention of this type is described in Example 3 with reference to FIG. 8B. Thus, for example, antibody ACI-31-25B1-Ab2 may be used as capture antibody and ACI-24-41F12-Ab2 used as detection antibody.

Some binding molecules of the invention may show high cross-reactivity with sAPPalpha. Again, this may be measured for example by comparing the binding to sAPPalpha with binding to Abeta1-42. When normalized against Abeta1-42 binding, the level of binding to sAPPalpha may be greater than 60%, such as between 70 and 90%, such as around 80%, of binding to Abeta1-42. Such comparison may be performed by ELISA, for example an indirect ELISA. A suitable assay is described in Example 3 with reference to FIG. 8A. Results are shown in Table 1 for the tested antibodies of the invention. Binding molecules with high levels of cross-reactivity to sAPPalpha may be used in combination with binding molecules with no cross-reactivity to sAPPalpha. Thus, the combination of a binding molecule with high levels of cross-reactivity to sAPPalpha and a binding molecule with no cross-reactivity to sAPPalpha may be utilized according to the invention for performing sandwich immunoassays, giving high sensitivity for Abeta, and no detection of soluble APPalpha (i.e. high specificity). In such an assay, the selectivity is provided by the binding molecule of the invention which does not cross-react with sAPPalpha. In particular, the binding molecule with high levels of cross-reactivity to sAPPalpha may be used as capture or detection antibody and the binding molecule with no cross-reactivity to sAPPalpha may be used as detection or capture antibody. Antibodies of the invention with high cross-reactivity to sAPPalpha include ACI-8037-103H5-Ab2 and ACI-8037-109F4-Ab1. In some embodiments, the binding molecule, in particular the antibody or the antigen-binding fragment thereof, comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 13; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 13; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VH-CDR1 comprising the amino acid sequence of SEQ ID NO: 31; VH-CDR2 comprising the amino acid sequence of SEQ ID NO: 32; and VH-CDR3 comprising the amino acid sequence of SEQ ID NO: 33; VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37. Thus, the invention also provides an amyloid-beta binding molecule, in particular an amyloid-beta antibody or antigen-binding fragment thereof, which comprises VH-CDR1 comprising the amino acid sequence of SEQ ID NO: 31; VH-CDR2 comprising the amino acid sequence of SEQ ID NO: 32; and VH-CDR3 comprising the amino acid sequence of SEQ ID NO: 33; VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37.

In some embodiments, the binding molecule, in particular the antibody or the antigen-binding fragment thereof, comprises VH-CDR1 comprising the amino acid sequence of SEQ ID NO: 41; VH-CDR2 comprising the amino acid sequence of SEQ ID NO: 42; and VH-CDR3 comprising the amino acid sequence of SEQ ID NO: 43; VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47. Thus, the invention also provides an amyloid-beta binding molecule, in particular an amyloid-beta antibody or antigen-binding fragment thereof, which comprises VH-CDR1 comprising the amino acid sequence of SEQ ID NO: 41; VH-CDR2 comprising the amino acid sequence of SEQ ID NO: 42; and VH-CDR3 comprising the amino acid sequence of SEQ ID NO: 43; VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47.

In some embodiments, the binding molecule, in particular the antibody or the antigen-binding fragment thereof, comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 63; VL-CDR1 comprising the amino acid sequence of SEQ ID NO: 65; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 63; VL-CDR1 comprising the amino acid sequence of SEQ ID NO: 65; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 73; VL-CDR1 comprising the amino acid sequence of SEQ ID NO: 75; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 73; VL-CDR1 comprising the amino acid sequence of SEQ ID NO: 75; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 83; VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87. Thus, the invention also provides an amyloid-beta binding molecule, in particular an amyloid-beta antibody or antigen-binding fragment thereof, which comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 83; VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87.

In some embodiments, the binding molecule, in particular the antibody or the antigen-binding fragment thereof, comprises VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97. Thus, the invention also provides an amyloid-beta binding molecule, in particular an amyloid-beta antibody or antigen-binding fragment thereof, which comprises VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97.

In some embodiments, the binding molecule, in particular the antibody or the antigen-binding fragment thereof, comprises VH-CDR1 comprising the amino acid sequence of SEQ ID NO: 101; VH-CDR2 comprising the amino acid sequence of SEQ ID NO: 102; and VH-CDR3 comprising the amino acid sequence of SEQ ID NO: 103; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107. Thus, the invention also provides an amyloid-beta binding molecule, in particular an amyloid-beta antibody or antigen-binding fragment thereof, which comprises VH-CDR1 comprising the amino acid sequence of SEQ ID NO: 101; VH-CDR2 comprising the amino acid sequence of SEQ ID NO: 102; and VH-CDR3 comprising the amino acid sequence of SEQ ID NO: 103; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107.

In some embodiments, the binding molecule, in particular the antibody or the antigen-binding fragment thereof, comprises VH-CDR1 comprising the amino acid sequence of SEQ ID NO: 111; VH-CDR2 comprising the amino acid sequence of SEQ ID NO: 112; and VH-CDR3 comprising the amino acid sequence of SEQ ID NO: 113; VL-CDR1 comprising the amino acid sequence of SEQ ID NO: 115; VL-CDR2 comprising the amino acid sequence of SEQ ID NO: 116; and VL-CDR3 comprising the amino acid sequence of SEQ ID NO: 117. Thus, the invention also provides an amyloid-beta binding molecule, in particular an amyloid-beta antibody or antigen-binding fragment thereof, which comprises VH-CDR1 comprising the amino acid sequence of SEQ ID NO: 111; VH-CDR2 comprising the amino acid sequence of SEQ ID NO: 112; and VH-CDR3 comprising the amino acid sequence of SEQ ID NO: 113; VL-CDR1 comprising the amino acid sequence of SEQ ID NO: 115; VL-CDR2 comprising the amino acid sequence of SEQ ID NO: 116; and VL-CDR3 comprising the amino acid sequence of SEQ ID NO: 117.

In some embodiments, the binding molecule, in particular the antibody or the antigen-binding fragment thereof, comprises VH-CDR1 comprising the amino acid sequence of SEQ ID NO: 121; VH-CDR2 comprising the amino acid sequence of SEQ ID NO: 122; and VH-CDR3 comprising the amino acid sequence of SEQ ID NO: 123; VL-CDR1 comprising the amino acid sequence of SEQ ID NO: 125; VL-CDR2 comprising the amino acid sequence of SEQ ID NO: 126; and VL-CDR3 comprising the amino acid sequence of SEQ ID NO: 127. Thus, the invention also provides an amyloid-beta binding molecule, in particular an amyloid-beta antibody or antigen-binding fragment thereof, which comprises VH-CDR1 comprising the amino acid sequence of SEQ ID NO: 121; VH-CDR2 comprising the amino acid sequence of SEQ ID NO: 122; and VH-CDR3 comprising the amino acid sequence of SEQ ID NO: 123; VL-CDR1 comprising the amino acid sequence of SEQ ID NO: 125; VL-CDR2 comprising the amino acid sequence of SEQ ID NO: 126; and VL-CDR3 comprising the amino acid sequence of SEQ ID NO: 127.

In some embodiments, the binding molecule, in particular the antibody or the antigen-binding fragment thereof, comprises VH-CDR1 comprising the amino acid sequence of SEQ ID NO: 131; VH-CDR2 comprising the amino acid sequence of SEQ ID NO: 132; and VH-CDR3 comprising the amino acid sequence of SEQ ID NO: 133; VL-CDR1 comprising the amino acid sequence of SEQ ID NO: 135; VL-CDR2 comprising the amino acid sequence of SEQ ID NO: 16; and VL-CDR3 comprising the amino acid sequence of SEQ ID NO: 137. Thus, the invention also provides an amyloid-beta binding molecule, in particular an amyloid-beta antibody or antigen-binding fragment thereof, which comprises VH-CDR1 comprising the amino acid sequence of SEQ ID NO: 131; VH-CDR2 comprising the amino acid sequence of SEQ ID NO: 132; and VH-CDR3 comprising the amino acid sequence of SEQ ID NO: 133; VL-CDR1 comprising the amino acid sequence of SEQ ID NO: 135; VL-CDR2 comprising the amino acid sequence of SEQ ID NO: 16; and VL-CDR3 comprising the amino acid sequence of SEQ ID NO: 137.

In some embodiments, the binding molecule, in particular the antibody or the antigen-binding fragment thereof, comprises VH-CDR1 comprising the amino acid sequence of SEQ ID NO: 141; VH-CDR2 comprising the amino acid sequence of SEQ ID NO: 142; and VH-CDR3 comprising the amino acid sequence of SEQ ID NO: 133; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 137. Thus, the invention also provides an amyloid-beta binding molecule, in particular an amyloid-beta antibody or antigen-binding fragment thereof, which comprises VH-CDR1 comprising the amino acid sequence of SEQ ID NO: 141; VH-CDR2 comprising the amino acid sequence of SEQ ID NO: 142; and VH-CDR3 comprising the amino acid sequence of SEQ ID NO: 133; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 137.

In some embodiments, the binding molecule, in particular the antibody or the antigen-binding fragment thereof, comprises VH-CDR1 comprising the amino acid sequence of SEQ ID NO: 151; VH-CDR2 comprising the amino acid sequence of SEQ ID NO: 152; and VH-CDR3 comprising the amino acid sequence of SEQ ID NO: 153; VL-CDR1 comprising the amino acid sequence of SEQ ID NO: 155; VL-CDR2 comprising the amino acid sequence of SEQ ID NO: 156; and VL-CDR3 comprising the amino acid sequence of SEQ ID NO: 157. Thus, the invention also provides an amyloid-beta binding molecule, in particular an amyloid-beta antibody or antigen-binding fragment thereof, which comprises VH-CDR1 comprising the amino acid sequence of SEQ ID NO: 151; VH-CDR2 comprising the amino acid sequence of SEQ ID NO: 152; and VH-CDR3 comprising the amino acid sequence of SEQ ID NO: 153; VL-CDR1 comprising the amino acid sequence of SEQ ID NO: 155; VL-CDR2 comprising the amino acid sequence of SEQ ID NO: 156; and VL-CDR3 comprising the amino acid sequence of SEQ ID NO: 157. In some embodiments, the binding molecule, in particular the antibody or the antigen-binding fragment thereof, comprises VH-CDR1 comprising the amino acid sequence of SEQ ID NO: 161; VH-CDR2 comprising the amino acid sequence of SEQ ID NO: 162; and VH-CDR3 comprising the amino acid sequence of SEQ ID NO: 163; VL-CDR1 comprising the amino acid sequence of SEQ ID NO: 165; VL-CDR2 comprising the amino acid sequence of SEQ ID NO: 166; and VL-CDR3 comprising the amino acid sequence of SEQ ID NO: 167. Thus, the invention also provides an amyloid-beta binding molecule, in particular an amyloid-beta antibody or antigen-binding fragment thereof, which comprises VH-CDR1 comprising the amino acid sequence of SEQ ID NO: 161; VH-CDR2 comprising the amino acid sequence of SEQ ID NO: 162; and VH-CDR3 comprising the amino acid sequence of SEQ ID NO: 163; VL-CDR1 comprising the amino acid sequence of SEQ ID NO: 165; VL-CDR2 comprising the amino acid sequence of SEQ ID NO: 166; and VL-CDR3 comprising the amino acid sequence of SEQ ID NO: 167.

In some embodiments, the binding molecule, in particular the antibody or the antigen-binding fragment thereof, comprises VH-CDR1 comprising the amino acid sequence of SEQ ID NO: 171; VH-CDR2 comprising the amino acid sequence of SEQ ID NO: 172; and VH-CDR3 comprising the amino acid sequence of SEQ ID NO: 173; VL-CDR1 comprising the amino acid sequence of SEQ ID NO: 175; VL-CDR2 comprising the amino acid sequence of SEQ ID NO: 156; and VL-CDR3 comprising the amino acid sequence of SEQ ID NO: 157. Thus, the invention also provides an amyloid-beta binding molecule, in particular an amyloid-beta antibody or antigen-binding fragment thereof, which comprises VH-CDR1 comprising the amino acid sequence of SEQ ID NO: 171; VH-CDR2 comprising the amino acid sequence of SEQ ID NO: 172; and VH-CDR3 comprising the amino acid sequence of SEQ ID NO: 173; VL-CDR1 comprising the amino acid sequence of SEQ ID NO: 175; VL-CDR2 comprising the amino acid sequence of SEQ ID NO: 156; and VL-CDR3 comprising the amino acid sequence of SEQ ID NO: 157.

In some embodiments, the binding molecule, in particular the antibody or the antigen-binding fragment thereof, comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 187. Thus, the invention also provides an amyloid-beta binding molecule, in particular an amyloid-beta antibody or antigen-binding fragment thereof, which comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 187.

In some embodiments, the binding molecule, in particular the antibody or the antigen-binding fragment thereof, comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 195; VL-CDR2 comprising the amino acid sequence of SEQ ID NO: 196; and VL-CDR3 comprising the amino acid sequence of SEQ ID NO: 197. Thus, the invention also provides an amyloid-beta binding molecule, in particular an amyloid-beta antibody or antigen-binding fragment thereof, which comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 195; VL-CDR2 comprising the amino acid sequence of SEQ ID NO: 196; and VL-CDR3 comprising the amino acid sequence of SEQ ID NO: 197.

In some embodiments, the binding molecule, in particular the antibody or the antigen-binding fragment thereof, comprises a Heavy Chain Variable Region (VH) comprising the sequence of SEQ ID NO: 10 and a Light Chain Variable Region (VL) comprising the sequence of SEQ ID NO: 14. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 10 and a Light Chain Variable Region (VL) comprising the sequence of SEQ ID NO: 14.

In some embodiments, the binding molecule, in particular the antibody or the antigen-binding fragment thereof, comprises a Heavy Chain Variable Region (VH) comprising the sequence of SEQ ID NO: 30 and a Light Chain Variable Region (VL) comprising the sequence of SEQ ID NO: 34. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 30 and a Light Chain Variable Region (VL) comprising the sequence of SEQ ID NO: 34.

In some embodiments, the binding molecule, in particular the antibody or the antigen-binding fragment thereof, comprises a Heavy Chain Variable Region (VH) comprising the sequence of SEQ ID NO: 40 and a Light Chain Variable Region (VL) comprising the sequence of SEQ ID NO: 44. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 40 and a Light Chain Variable Region (VL) comprising the sequence of SEQ ID NO: 44.

In some embodiments, the binding molecule, in particular the antibody or the antigen-binding fragment thereof, comprises a Heavy Chain Variable Region (VH) comprising the sequence of SEQ ID NO: 60 and a Light Chain Variable Region (VL) comprising the sequence of SEQ ID NO: 64. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 60 and a Light Chain Variable Region (VL) comprising the sequence of SEQ ID NO: 64.

In some embodiments, the binding molecule, in particular the antibody or the antigen-binding fragment thereof, comprises a Heavy Chain Variable Region (VH) comprising the sequence of SEQ ID NO: 70 and a Light Chain Variable Region (VL) comprising the sequence of SEQ ID NO: 74. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 70 and a Light Chain Variable Region (VL) comprising the sequence of SEQ ID NO: 74.

In some embodiments, the binding molecule, in particular the antibody or the antigen-binding fragment thereof, comprises a Heavy Chain Variable Region (VH) comprising the sequence of SEQ ID NO: 80 and a Light Chain Variable Region (VL) comprising the sequence of SEQ ID NO: 84. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 80 and a Light Chain Variable Region (VL) comprising the sequence of SEQ ID NO: 84.

In some embodiments, the binding molecule, in particular the antibody or the antigen-binding fragment thereof, comprises a Heavy Chain Variable Region (VH) comprising the sequence of SEQ ID NO: 90 and a Light Chain Variable Region (VL) comprising the sequence of SEQ ID NO: 94. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 90 and a Light Chain Variable Region (VL) comprising the sequence of SEQ ID NO: 94.

In some embodiments, the binding molecule, in particular the antibody or the antigen-binding fragment thereof, comprises a Heavy Chain Variable Region (VH) comprising the sequence of SEQ ID NO: 100 and a Light Chain Variable Region (VL) comprising the sequence of SEQ ID NO: 104. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 100 and a Light Chain Variable Region (VL) comprising the sequence of SEQ ID NO: 104.

In some embodiments, the binding molecule, in particular the antibody or the antigen-binding fragment thereof, comprises a Heavy Chain Variable Region (VH) comprising the sequence of SEQ ID NO: 110 and a Light Chain Variable Region (VL) comprising the sequence of SEQ ID NO: 114. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 110 and a Light Chain Variable Region (VL) comprising the sequence of SEQ ID NO: 114.

In some embodiments, the binding molecule, in particular the antibody or the antigen-binding fragment thereof, comprises a Heavy Chain Variable Region (VH) comprising the sequence of SEQ ID NO: 120 and a Light Chain Variable Region (VL) comprising the sequence of SEQ ID NO: 124. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 120 and a Light Chain Variable Region (VL) comprising the sequence of SEQ ID NO: 124.

In some embodiments, the binding molecule, in particular the antibody or the antigen-binding fragment thereof, comprises a Heavy Chain Variable Region (VH) comprising the sequence of SEQ ID NO: 130 and a Light Chain Variable Region (VL) comprising the sequence of SEQ ID NO: 134. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 130 and a Light Chain Variable Region (VL) comprising the sequence of SEQ ID NO: 134.

In some embodiments, the binding molecule, in particular the antibody or the antigen-binding fragment thereof, comprises a Heavy Chain Variable Region (VH) comprising the sequence of SEQ ID NO: 140 and a Light Chain Variable Region (VL) comprising the sequence of SEQ ID NO: 144. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 140 and a Light Chain Variable Region (VL) comprising the sequence of SEQ ID NO: 144.

In some embodiments, the binding molecule, in particular the antibody or the antigen-binding fragment thereof, comprises a Heavy Chain Variable Region (VH) comprising the sequence of SEQ ID NO: 150 and a Light Chain Variable Region (VL) comprising the sequence of SEQ ID NO: 154. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 150 and a Light Chain Variable Region (VL) comprising the sequence of SEQ ID NO: 154.

In some embodiments, the binding molecule, in particular the antibody or the antigen-binding fragment thereof, comprises a Heavy Chain Variable Region (VH) comprising the sequence of SEQ ID NO: 160 and a Light Chain Variable Region (VL) comprising the sequence of SEQ ID NO: 164. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 160 and a Light Chain Variable Region (VL) comprising the sequence of SEQ ID NO: 164.

In some embodiments, the binding molecule, in particular the antibody or the antigen-binding fragment thereof, comprises a Heavy Chain Variable Region (VH) comprising the sequence of SEQ ID NO: 170 and a Light Chain Variable Region (VL) comprising the sequence of SEQ ID NO: 174. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 170 and a Light Chain Variable Region (VL) comprising the sequence of SEQ ID NO: 174.

In some embodiments, the binding molecule, in particular the antibody or the antigen-binding fragment thereof, comprises a Heavy Chain Variable Region (VH) comprising the sequence of SEQ ID NO: 180 and a Light Chain Variable Region (VL) comprising the sequence of SEQ ID NO: 184. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 180 and a Light Chain Variable Region (VL) comprising the sequence of SEQ ID NO: 184.

In some embodiments, the binding molecule, in particular the antibody or the antigen-binding fragment thereof, comprises a Heavy Chain Variable Region (VH) comprising the sequence of SEQ ID NO: 180 and a Light Chain Variable Region (VL) comprising the sequence of SEQ ID NO: 194. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 180 and a Light Chain Variable Region (VL) comprising the sequence of SEQ ID NO: 194.

In some embodiments, the binding molecule, in particular the antibody or the antigen-binding fragment thereof, comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14.

In some embodiments, the binding molecule, in particular the antibody or the antigen-binding fragment thereof, comprises a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34.

In some embodiments, the binding molecule, in particular the antibody or the antigen-binding fragment thereof, comprises a Heavy Chain Variable Region (VH) comprising the sequence of SEQ ID NO: 40 or a Heavy Chain Variable Region (VH) having at least 93%, 94%, 95%, 96%, 97%, 98%, or 99% sequence identity to the amino acid sequence of SEQ ID NO: 40; and a Light Chain Variable Region (VL) comprising the sequence of SEQ ID NO: 44 or a Light Chain Variable Region (VL) having at least 97%, 98%, or 99% sequence identity to the amino acid sequence of SEQ ID NO: 44. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 40 or a Heavy Chain Variable Region (VH) having at least 93%, 94%, 95%, 96%, 97%, 98%, or 99% sequence identity to the amino acid sequence of SEQ ID NO: 40; and a Light Chain Variable Region (VL) comprising the sequence of SEQ ID NO: 44 or a Light Chain Variable Region (VL) having at least 97%, 98%, or 99% sequence identity to the amino acid sequence of SEQ ID NO: 44.

In some embodiments, the binding molecule, in particular the antibody or the antigen-binding fragment thereof, comprises a Heavy Chain Variable Region (VH) comprising the sequence of SEQ ID NO: 60 or a Heavy Chain Variable Region (VH) having at least 98%, or 99% sequence identity to the amino acid sequence of SEQ ID NO: 60; and a Light Chain Variable Region (VL) comprising the sequence of SEQ ID NO: 64 or a Light Chain Variable Region (VL) having at least 98%, or 99% sequence identity to the amino acid sequence of SEQ ID NO: 64. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 60 or a Heavy Chain Variable Region (VH) having at least 98%, or 99% sequence identity to the amino acid sequence of SEQ ID NO: 60; and a Light Chain Variable Region (VL) comprising the sequence of SEQ ID NO: 64 or a Light Chain Variable Region (VL) having at least 98%, or 99% sequence identity to the amino acid sequence of SEQ ID NO: 64.

In some embodiments, the binding molecule, in particular the antibody or the antigen-binding fragment thereof, comprises a Heavy Chain Variable Region (VH) comprising the sequence of SEQ ID NO: 70 or a Heavy Chain Variable Region (VH) having at least 98%, or 99% sequence identity to the amino acid sequence of SEQ ID NO: 70; and a Light Chain Variable Region (VL) comprising the sequence of SEQ ID NO: 74 or a Light Chain Variable Region (VL) having at least 99% sequence identity to the amino acid sequence of SEQ ID NO: 74. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 70 or a Heavy Chain Variable Region (VH) having at least 98%, or 99% sequence identity to the amino acid sequence of SEQ ID NO: 70; and a Light Chain Variable Region (VL) comprising the sequence of SEQ ID NO: 74 or a Light Chain Variable Region (VL) having at least 99% sequence identity to the amino acid sequence of SEQ ID NO: 74.

In some embodiments, the binding molecule, in particular the antibody or the antigen-binding fragment thereof, comprises a Heavy Chain Variable Region (VH) comprising the sequence of SEQ ID NO: 80 or a Heavy Chain Variable Region (VH) having at least 99% sequence identity to the amino acid sequence of SEQ ID NO: 80; and a Light Chain Variable Region (VL) comprising the sequence of SEQ ID NO: 84. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 80 or a Heavy Chain Variable Region (VH) having at least 99% sequence identity to the amino acid sequence of SEQ ID NO: 80; and a Light Chain Variable Region (VL) comprising the sequence of SEQ ID NO: 84.

In some embodiments, the binding molecule, in particular the antibody or the antigen-binding fragment thereof, comprises a Heavy Chain Variable Region (VH) comprising the sequence of SEQ ID NO: 90 or a Heavy Chain Variable Region (VH) having at least 97%, 98%, or 99% sequence identity to the amino acid sequence of SEQ ID NO: 90; and a Light Chain Variable Region (VL) comprising the sequence of SEQ ID NO: 94 or a Light Chain Variable Region (VL) having at least 98%, or 99% sequence identity to the amino acid sequence of SEQ ID NO: 94. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 90 or a Heavy Chain Variable Region (VH) having at least 97%, 98%, or 99% sequence identity to the amino acid sequence of SEQ ID NO: 90; and a Light Chain Variable Region (VL) comprising the sequence of SEQ ID NO: 94 or a Light Chain Variable Region (VL) having at least 98%, or 99% sequence identity to the amino acid sequence of SEQ ID NO: 94.

In some embodiments, the binding molecule, in particular the antibody or the antigen-binding fragment thereof, comprises a Heavy Chain Variable Region (VH) comprising the sequence of SEQ ID NO: 100 or a Heavy Chain Variable Region (VH) having at least 99% sequence identity to the amino acid sequence of SEQ ID NO: 100; and a Light Chain Variable Region (VL) comprising the sequence of SEQ ID NO: 104. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 100 or a Heavy Chain Variable Region (VH) having at least 99% sequence identity to the amino acid sequence of SEQ ID NO: 100; and a Light Chain Variable Region (VL) comprising the sequence of SEQ ID NO: 104.

In some embodiments, the binding molecule, in particular the antibody or the antigen-binding fragment thereof, comprises a Heavy Chain Variable Region (VH) comprising the sequence of SEQ ID NO: 110 or a Heavy Chain Variable Region (VH) having at least 97%, 98%, or 99% sequence identity to the amino acid sequence of SEQ ID NO: 110; and a Light Chain Variable Region (VL) comprising the sequence of SEQ ID NO: 114 or a Light Chain Variable Region (VL) having at least 98%, or 99% sequence identity to the amino acid sequence of SEQ ID NO: 114. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 110 or a Heavy Chain Variable Region (VH) having at least 97%, 98%, or 99% sequence identity to the amino acid sequence of SEQ ID NO: 110; and a Light Chain Variable Region (VL) comprising the sequence of SEQ ID NO: 114 or a Light Chain Variable Region (VL) having at least 98%, or 99% sequence identity to the amino acid sequence of SEQ ID NO: 114.

In some embodiments, the binding molecule, in particular the antibody or the antigen-binding fragment thereof, comprises a Heavy Chain Variable Region (VH) comprising the sequence of SEQ ID NO: 120 or a Heavy Chain Variable Region (VH) having at least 95%, 96%, 97%, 98%, or 99% sequence identity to the amino acid sequence of SEQ ID NO: 120; and a Light Chain Variable Region (VL) comprising the sequence of SEQ ID NO: 124 or a Light Chain Variable Region (VL) having at least 99% sequence identity to the amino acid sequence of SEQ ID NO: 124. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 120 or a Heavy Chain Variable Region (VH) having at least 95%, 96%, 97%, 98%, or 99% sequence identity to the amino acid sequence of SEQ ID NO: 120; and a Light Chain Variable Region (VL) comprising the sequence of SEQ ID NO: 124 or a Light Chain Variable Region (VL) having at least 99% sequence identity to the amino acid sequence of SEQ ID NO: 124.

In some embodiments, the binding molecule, in particular the antibody or the antigen-binding fragment thereof, comprises a Heavy Chain Variable Region (VH) comprising the sequence of SEQ ID NO: 130 or a Heavy Chain Variable Region (VH) having at least 94%, 95%, 96%, 97%, 98%, or 99% sequence identity to the amino acid sequence of SEQ ID NO: 130; and a Light Chain Variable Region (VL) comprising the sequence of SEQ ID NO: 134. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 130 or a Heavy Chain Variable Region (VH) having at least 94%, 95%, 96%, 97%, 98%, or 99% sequence identity to the amino acid sequence of SEQ ID NO: 130; and a Light Chain Variable Region (VL) comprising the sequence of SEQ ID NO: 134.

In some embodiments, the binding molecule, in particular the antibody or the antigen-binding fragment thereof, comprises a Heavy Chain Variable Region (VH) comprising the sequence of SEQ ID NO: 140 or a Heavy Chain Variable Region (VH) having at least 96%, 97%, 98%, or 99% sequence identity to the amino acid sequence of SEQ ID NO: 140; and a Light Chain Variable Region (VL) comprising the sequence of SEQ ID NO:144. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 140 or a Heavy Chain Variable Region (VH) having at least 96%, 97%, 98%, or 99% sequence identity to the amino acid sequence of SEQ ID NO: 140; and a Light Chain Variable Region (VL) comprising the sequence of SEQ ID NO: 144.

In some embodiments, the binding molecule, in particular the antibody or the antigen-binding fragment thereof, comprises a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154.

In some embodiments, the binding molecule, in particular the antibody or the antigen-binding fragment thereof, comprises a Heavy Chain Variable Region (VH) comprising the sequence of SEQ ID NO: 160 or a Heavy Chain Variable Region (VH) having at least 97%, 98%, or 99% sequence identity to the amino acid sequence of SEQ ID NO: 160; and a Light Chain Variable Region (VL) comprising the sequence of SEQ ID NO: 164 or a Light Chain Variable Region (VL) having at least 99% sequence identity to the amino acid sequence of SEQ ID NO: 164. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 160 or a Heavy Chain Variable Region (VH) having at least 97%, 98%, or 99% sequence identity to the amino acid sequence of SEQ ID NO: 160; and a Light Chain Variable Region (VL) comprising the sequence of SEQ ID NO: 164 or a Light Chain Variable Region (VL) having at least 99% sequence identity to the amino acid sequence of SEQ ID NO: 164.

In some embodiments, the binding molecule, in particular the antibody or the antigen-binding fragment thereof, comprises a Heavy Chain Variable Region (VH) comprising the sequence of SEQ ID NO: 170 or a Heavy Chain Variable Region (VH) having at least 92%, 93%, 94%, 95%, 96%, 97%, 98%, or 99% sequence identity to the amino acid sequence of SEQ ID NO: 170; and a Light Chain Variable Region (VL) comprising the sequence of SEQ ID NO: 174. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 170 or a Heavy Chain Variable Region (VH) having at least 92%, 93%, 94%, 95%, 96%, 97%, 98%, or 99% sequence identity to the amino acid sequence of SEQ ID NO: 170; and a Light Chain Variable Region (VL) comprising the sequence of SEQ ID NO: 174.

In some embodiments, the binding molecule, in particular the antibody or the antigen-binding fragment thereof, comprises a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 184. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 184.

In some embodiments, the binding molecule, in particular the antibody or the antigen-binding fragment thereof, comprises a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 194. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 194.

In some embodiments, the binding molecule, in particular the antibody or the antigen-binding fragment thereof, comprises a Heavy Chain Variable Region (VH) comprising the sequence of SEQ ID NO: 10 or a Heavy Chain Variable Region (VH) having at least 96%, 97%, 98%, or 99%, sequence identity to the amino acid sequence of SEQ ID NO: 10. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 10 or a Heavy Chain Variable Region (VH) having at least 96%, 97%, 98%, or 99%, sequence identity to the amino acid sequence of SEQ ID NO: 10.

In some embodiments, the binding molecule, in particular the antibody or the antigen-binding fragment thereof, comprises a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99%, sequence identity to the amino acid sequence of SEQ ID NO: 30. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99%, sequence identity to the amino acid sequence of SEQ ID NO: 30.

In some embodiments, the binding molecule, in particular the antibody or the antigen-binding fragment thereof, comprises a Heavy Chain Variable Region (VH) comprising the sequence of SEQ ID NO: 40 or a Heavy Chain Variable Region (VH) having at least 93%, 94%, 95%, 96%, 97%, 98%, or 99%, sequence identity to the amino acid sequence of SEQ ID NO: 40. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 40 or a Heavy Chain Variable Region (VH) having at least 93%, 94%, 95%, 96%, 97%, 98%, or 99%, sequence identity to the amino acid sequence of SEQ ID NO: 40.

In some embodiments, the binding molecule, in particular the antibody or the antigen-binding fragment thereof, comprises a Heavy Chain Variable Region (VH) comprising the sequence of SEQ ID NO: 60 or a Heavy Chain Variable Region (VH) having at least 98%, or 99%, sequence identity to the amino acid sequence of SEQ ID NO: 60. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 60 or a Heavy Chain Variable Region (VH) having at least 98%, or 99%, sequence identity to the amino acid sequence of SEQ ID NO: 60.

In some embodiments, the binding molecule, in particular the antibody or the antigen-binding fragment thereof, comprises a Heavy Chain Variable Region (VH) comprising the sequence of SEQ ID NO: 70 or a Heavy Chain Variable Region (VH) having at least 98%, or 99%, sequence identity to the amino acid sequence of SEQ ID NO: 70. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 70 or a Heavy Chain Variable Region (VH) having at least 98%, or 99%, sequence identity to the amino acid sequence of SEQ ID NO: 70.

In some embodiments, the binding molecule, in particular the antibody or the antigen-binding fragment thereof, comprises a Heavy Chain Variable Region (VH) comprising the sequence of SEQ ID NO: 80 or a Heavy Chain Variable Region (VH) having at least 99%, sequence identity to the amino acid sequence of SEQ ID NO: 80. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 80 or a Heavy Chain Variable Region (VH) having at least 99%, sequence identity to the amino acid sequence of SEQ ID NO: 80.

In some embodiments, the binding molecule, in particular the antibody or the antigen-binding fragment thereof, comprises a Heavy Chain Variable Region (VH) comprising the sequence of SEQ ID NO: 90 or a Heavy Chain Variable Region (VH) having at least 97%, 98%, or 99%, sequence identity to the amino acid sequence of SEQ ID NO: 90. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 90 or a Heavy Chain Variable Region (VH) having at least 97%, 98%, or 99%, sequence identity to the amino acid sequence of SEQ ID NO: 90.

In some embodiments, the binding molecule, in particular the antibody or the antigen-binding fragment thereof, comprises a Heavy Chain Variable Region (VH) comprising the sequence of SEQ ID NO: 100 or a Heavy Chain Variable Region (VH) having at least 99%, sequence identity to the amino acid sequence of SEQ ID NO: 100. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 100 or a Heavy Chain Variable Region (VH) having at least 99%, sequence identity to the amino acid sequence of SEQ ID NO: 100.

In some embodiments, the binding molecule, in particular the antibody or the antigen-binding fragment thereof, comprises a Heavy Chain Variable Region (VH) comprising the sequence of SEQ ID NO: 110 or a Heavy Chain Variable Region (VH) having at least 97%, 98%, or 99%, sequence identity to the amino acid sequence of SEQ ID NO: 110. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 110 or a Heavy Chain Variable Region (VH) having at least 97%, 98%, or 99%, sequence identity to the amino acid sequence of SEQ ID NO: 110.

In some embodiments, the binding molecule, in particular the antibody or the antigen-binding fragment thereof, comprises a Heavy Chain Variable Region (VH) comprising the sequence of SEQ ID NO: 120 or a Heavy Chain Variable Region (VH) having at least 95%, 96%, 97%, 98%, or 99%, sequence identity to the amino acid sequence of SEQ ID NO: 120. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 120 or a Heavy Chain Variable Region (VH) having at least 95%, 96%, 97%, 98%, or 99%, sequence identity to the amino acid sequence of SEQ ID NO: 120.

In some embodiments, the binding molecule, in particular the antibody or the antigen-binding fragment thereof, comprises a Heavy Chain Variable Region (VH) comprising the sequence of SEQ ID NO: 130 or a Heavy Chain Variable Region (VH) having at least 94%, 95%, 96%, 97%, 98%, or 99%, sequence identity to the amino acid sequence of SEQ ID NO: 130. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 130 or a Heavy Chain Variable Region (VH) having at least 94%, 95%, 96%, 97%, 98%, or 99%, sequence identity to the amino acid sequence of SEQ ID NO: 130.

In some embodiments, the binding molecule, in particular the antibody or the antigen-binding fragment thereof, comprises a Heavy Chain Variable Region (VH) comprising the sequence of SEQ ID NO: 140 or a Heavy Chain Variable Region (VH) having at least 96%, 97%, 98%, or 99%, sequence identity to the amino acid sequence of SEQ ID NO: 140. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 140 or a Heavy Chain Variable Region (VH) having at least 96%, 97%, 98%, or 99%, sequence identity to the amino acid sequence of SEQ ID NO: 140.

In some embodiments, the binding molecule, in particular the antibody or the antigen-binding fragment thereof, comprises a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99%, sequence identity to the amino acid sequence of SEQ ID NO: 150. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99%, sequence identity to the amino acid sequence of SEQ ID NO: 150.

In some embodiments, the binding molecule, in particular the antibody or the antigen-binding fragment thereof, comprises a Heavy Chain Variable Region (VH) comprising the sequence of SEQ ID NO: 160 or a Heavy Chain Variable Region (VH) having at least 97%, 98%, or 99%, sequence identity to the amino acid sequence of SEQ ID NO: 160. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 160 or a Heavy Chain Variable Region (VH) having at least 97%, 98%, or 99%, sequence identity to the amino acid sequence of SEQ ID NO: 160.

In some embodiments, the binding molecule, in particular the antibody or the antigen-binding fragment thereof, comprises a Heavy Chain Variable Region (VH) comprising the sequence of SEQ ID NO: 170 or a Heavy Chain Variable Region (VH) having at least 92%, 93%, 94%, 95%, 96%, 97%, 98%, or 99%, sequence identity to the amino acid sequence of SEQ ID NO: 170. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 170 or a Heavy Chain Variable Region (VH) having at least 92%, 93%, 94%, 95%, 96%, 97%, 98%, or 99%, sequence identity to the amino acid sequence of SEQ ID NO: 170.

In some embodiments, the binding molecule, in particular the antibody or the antigen-binding fragment thereof, comprises a Heavy Chain Variable Region (VH) comprising the sequence of SEQ ID NO: 180 or a Heavy Chain Variable Region (VH) having at least 94%, 95%, 96%, 97%, 98%, or 99%, sequence identity to the amino acid sequence of SEQ ID NO: 180. Thus, the invention also provides an amyloid-beta binding molecule, in particular an amyloid-beta antibody or antigen-binding fragment thereof, which comprises a Heavy Chain Variable Region (VH) comprising the sequence of SEQ ID NO: 180 or a Heavy Chain Variable Region (VH) having at least 94%, 95%, 96%, 97%, 98%, or 99%, sequence identity to the amino acid sequence of SEQ ID NO: 180.

In some embodiments, the binding molecule, in particular the antibody or the antigen-binding fragment thereof, comprises a Light Chain Variable Region (VL) comprising the sequence of SEQ ID NO: 14. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 14.

In some embodiments, the binding molecule, in particular the antibody or the antigen-binding fragment thereof, comprises a Light Chain Variable Region (VL) comprising the sequence of SEQ ID NO: 34 or a Light Chain Variable Region (VL) having at least 96%, 97%, 98%, or 99%, sequence identity to the amino acid sequence of SEQ ID NO: 34. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 34 or a Light Chain Variable Region (VL) having at least 96%, 97%, 98%, or 99%, sequence identity to the amino acid sequence of SEQ ID NO: 34.

In some embodiments, the binding molecule, in particular the antibody or the antigen-binding fragment thereof, comprises a Light Chain Variable Region (VL) comprising the sequence of SEQ ID NO: 44 or a Light Chain Variable Region (VL) having at least 97%, 98%, or 99%, sequence identity to the amino acid sequence of SEQ ID NO: 44. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 44 or a Light Chain Variable Region (VL) having at least 97%, 98%, or 99%, sequence identity to the amino acid sequence of SEQ ID NO: 44.

In some embodiments, the binding molecule, in particular the antibody or the antigen-binding fragment thereof, comprises a Light Chain Variable Region (VL) comprising the sequence of SEQ ID NO: 64 or a Light Chain Variable Region (VL) having at least 98%, or 99%, sequence identity to the amino acid sequence of SEQ ID NO: 64. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 64 or a Light Chain Variable Region (VL) having at least 98%, or 99%, sequence identity to the amino acid sequence of SEQ ID NO: 64.

In some embodiments, the binding molecule, in particular the antibody or the antigen-binding fragment thereof, comprises a Light Chain Variable Region (VL) comprising the sequence of SEQ ID NO: 74 or a Light Chain Variable Region (VL) having at least 99%, sequence identity to the amino acid sequence of SEQ ID NO: 74. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 74 or a Light Chain Variable Region (VL) having at least 99%, sequence identity to the amino acid sequence of SEQ ID NO: 74.

In some embodiments, the binding molecule, in particular the antibody or the antigen-binding fragment thereof, comprises a Light Chain Variable Region (VL) comprising the sequence of SEQ ID NO: 84. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 84.

In some embodiments, the binding molecule, in particular the antibody or the antigen-binding fragment thereof, comprises a Light Chain Variable Region (VL) comprising the sequence of SEQ ID NO: 94 or a Light Chain Variable Region (VL) having at least 98%, or 99%, sequence identity to the amino acid sequence of SEQ ID NO: 94. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 94 or a Light Chain Variable Region (VL) having at least 98%, or 99%, sequence identity to the amino acid sequence of SEQ ID NO: 94.

In some embodiments, the binding molecule, in particular the antibody or the antigen-binding fragment thereof, comprises a Light Chain Variable Region (VL) comprising the sequence of SEQ ID NO: 104. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 104.

In some embodiments, the binding molecule, in particular the antibody or the antigen-binding fragment thereof, comprises a Light Chain Variable Region (VL) comprising the sequence of SEQ ID NO: 114 or a Light Chain Variable Region (VL) having at least 98%, or 99%, sequence identity to the amino acid sequence of SEQ ID NO: 114. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 114 or a Light Chain Variable Region (VL) having at least 98%, or 99%, sequence identity to the amino acid sequence of SEQ ID NO: 114.

In some embodiments, the binding molecule, in particular the antibody or the antigen-binding fragment thereof, comprises a Light Chain Variable Region (VL) comprising the sequence of SEQ ID NO: 124 or a Light Chain Variable Region (VL) having at least 99%, sequence identity to the amino acid sequence of SEQ ID NO: 124. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 124 or a Light Chain Variable Region (VL) having at least 99%, sequence identity to the amino acid sequence of SEQ ID NO: 124.

In some embodiments, the binding molecule, in particular the antibody or the antigen-binding fragment thereof, comprises a Light Chain Variable Region (VL) comprising the sequence of SEQ ID NO: 134. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 134.

In some embodiments, the binding molecule, in particular the antibody or the antigen-binding fragment thereof, comprises a Light Chain Variable Region (VL) comprising the sequence of SEQ ID NO: 144. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 144.

In some embodiments, the binding molecule, in particular the antibody or the antigen-binding fragment thereof, comprises a Light Chain Variable Region (VL) comprising the sequence of SEQ ID NO: 154 or a Light Chain Variable Region (VL) having at least 98%, or 99%, sequence identity to the amino acid sequence of SEQ ID NO: 154. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 154 or a Light Chain Variable Region (VL) having at least 98%, or 99%, sequence identity to the amino acid sequence of SEQ ID NO: 154.

In some embodiments, the binding molecule, in particular the antibody or the antigen-binding fragment thereof, comprises a Light Chain Variable Region (VL) comprising the sequence of SEQ ID NO: 164 or a Light Chain Variable Region (VL) having at least 99%, sequence identity to the amino acid sequence of SEQ ID NO: 164. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 164 or a Light Chain Variable Region (VL) having at least 99%, sequence identity to the amino acid sequence of SEQ ID NO: 164.

In some embodiments, the binding molecule, in particular the antibody or the antigen-binding fragment thereof, comprises a Light Chain Variable Region (VL) comprising the sequence of SEQ ID NO: 174. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 174.

In some embodiments, the binding molecule, in particular the antibody or the antigen-binding fragment thereof, comprises a Light Chain Variable Region (VL) comprising the sequence of SEQ ID NO: 184. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 184.

In some embodiments, the binding molecule, in particular the antibody or the antigen-binding fragment thereof, comprises a Light Chain Variable Region (VL) comprising the sequence of SEQ ID NO: 194. Thus, the invention also provides an amyloid-beta binding molecule, in particular an amyloid-beta antibody or antigen-binding fragment thereof, which comprises a Light Chain Variable Region (VL) comprising the sequence of SEQ ID NO: 194.

In some embodiments, the binding molecule, in particular the antibody or the antigen-binding fragment thereof, comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 13. Thus, the invention also provides an amyloid-beta binding molecule, in particular an amyloid-beta antibody or antigen-binding fragment thereof, which comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 13.

In some embodiments, the binding molecule, in particular the antibody or the antigen-binding fragment thereof, comprises VH-CDR1 comprising the amino acid sequence of SEQ ID NO: 31; VH-CDR2 comprising the amino acid sequence of SEQ ID NO: 32; and VH-CDR3 comprising the amino acid sequence of SEQ ID NO: 33. Thus, the invention also provides an amyloid-beta binding molecule, in particular an amyloid-beta antibody or antigen-binding fragment thereof, which comprises VH-CDR1 comprising the amino acid sequence of SEQ ID NO: 31; VH-CDR2 comprising the amino acid sequence of SEQ ID NO: 32; and VH-CDR3 comprising the amino acid sequence of SEQ ID NO: 33.

In some embodiments, the binding molecule, in particular the antibody or the antigen-binding fragment thereof, comprises VH-CDR1 comprising the amino acid sequence of SEQ ID NO: 41; VH-CDR2 comprising the amino acid sequence of SEQ ID NO: 42; and VH-CDR3 comprising the amino acid sequence of SEQ ID NO: 43. Thus, the invention also provides an amyloid-beta binding molecule, in particular an amyloid-beta antibody or antigen-binding fragment thereof, which comprises VH-CDR1 comprising the amino acid sequence of SEQ ID NO: 41; VH-CDR2 comprising the amino acid sequence of SEQ ID NO: 42; and VH-CDR3 comprising the amino acid sequence of SEQ ID NO: 43.

In some embodiments, the binding molecule, in particular the antibody or the antigen-binding fragment thereof, comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 63. Thus, the invention also provides an amyloid-beta binding molecule, in particular an amyloid-beta antibody or antigen-binding fragment thereof, which comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 63.

In some embodiments, the binding molecule, in particular the antibody or the antigen-binding fragment thereof, comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 73. Thus, the invention also provides an amyloid-beta binding molecule, in particular an amyloid-beta antibody or antigen-binding fragment thereof, which comprises VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 73.

In some embodiments, the binding molecule, in particular the antibody or the antigen-binding fragment thereof, comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 83. Thus, the invention also provides an amyloid-beta binding molecule, in particular an amyloid-beta antibody or antigen-binding fragment thereof, which comprises VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 83.

In some embodiments, the binding molecule, in particular the antibody or the antigen-binding fragment thereof, comprises VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93. Thus, the invention also provides an amyloid-beta binding molecule, in particular an amyloid-beta antibody or antigen-binding fragment thereof, which comprises VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93.

In some embodiments, the binding molecule, in particular the antibody or the antigen-binding fragment thereof, comprises VH-CDR1 comprising the amino acid sequence of SEQ ID NO: 101; VH-CDR2 comprising the amino acid sequence of SEQ ID NO: 102; and VH-CDR3 comprising the amino acid sequence of SEQ ID NO: 103. Thus, the invention also provides an amyloid-beta binding molecule, in particular an amyloid-beta antibody or antigen-binding fragment thereof, which comprises VH-CDR1 comprising the amino acid sequence of SEQ ID NO: 101; VH-CDR2 comprising the amino acid sequence of SEQ ID NO: 102; and VH-CDR3 comprising the amino acid sequence of SEQ ID NO: 103.

In some embodiments, the binding molecule, in particular the antibody or the antigen-binding fragment thereof, comprises VH-CDR1 comprising the amino acid sequence of SEQ ID NO: 111; VH-CDR2 comprising the amino acid sequence of SEQ ID NO: 112; and VH-CDR3 comprising the amino acid sequence of SEQ ID NO: 113. Thus, the invention also provides an amyloid-beta binding molecule, in particular an amyloid-beta antibody or antigen-binding fragment thereof, which comprises VH-CDR1 comprising the amino acid sequence of SEQ ID NO: 111; VH-CDR2 comprising the amino acid sequence of SEQ ID NO: 112; and VH-CDR3 comprising the amino acid sequence of SEQ ID NO: 113.

In some embodiments, the binding molecule, in particular the antibody or the antigen-binding fragment thereof, comprises VH-CDR1 comprising the amino acid sequence of SEQ ID NO: 121; VH-CDR2 comprising the amino acid sequence of SEQ ID NO: 122; and VH-CDR3 comprising the amino acid sequence of SEQ ID NO: 123. Thus, the invention also provides an amyloid-beta binding molecule, in particular an amyloid-beta antibody or antigen-binding fragment thereof, which comprises VH-CDR1 comprising the amino acid sequence of SEQ ID NO: 121; VH-CDR2 comprising the amino acid sequence of SEQ ID NO: 122; and VH-CDR3 comprising the amino acid sequence of SEQ ID NO: 123.

In some embodiments, the binding molecule, in particular the antibody or the antigen-binding fragment thereof, comprises VH-CDR1 comprising the amino acid sequence of SEQ ID NO: 131; VH-CDR2 comprising the amino acid sequence of SEQ ID NO: 132; and VH-CDR3 comprising the amino acid sequence of SEQ ID NO: 133. Thus, the invention also provides an amyloid-beta binding molecule, in particular an amyloid-beta antibody or antigen-binding fragment thereof, which comprises VH-CDR1 comprising the amino acid sequence of SEQ ID NO: 131; VH-CDR2 comprising the amino acid sequence of SEQ ID NO: 132; and VH-CDR3 comprising the amino acid sequence of SEQ ID NO: 133.

In some embodiments, the binding molecule, in particular the antibody or the antigen-binding fragment thereof, comprises VH-CDR1 comprising the amino acid sequence of SEQ ID NO: 141; VH-CDR2 comprising the amino acid sequence of SEQ ID NO: 142; and VH-CDR3 comprising the amino acid sequence of SEQ ID NO: 133. Thus, the invention also provides an amyloid-beta binding molecule, in particular an amyloid-beta antibody or antigen-binding fragment thereof, which comprises VH-CDR1 comprising the amino acid sequence of SEQ ID NO: 141; VH-CDR2 comprising the amino acid sequence of SEQ ID NO: 142; and VH-CDR3 comprising the amino acid sequence of SEQ ID NO: 133.

In some embodiments, the binding molecule, in particular the antibody or the antigen-binding fragment thereof, comprises VH-CDR1 comprising the amino acid sequence of SEQ ID NO: 151; VH-CDR2 comprising the amino acid sequence of SEQ ID NO: 152; and VH-CDR3 comprising the amino acid sequence of SEQ ID NO: 153. Thus, the invention also provides an amyloid-beta binding molecule, in particular an amyloid-beta antibody or antigen-binding fragment thereof, which comprises VH-CDR1 comprising the amino acid sequence of SEQ ID NO: 151; VH-CDR2 comprising the amino acid sequence of SEQ ID NO: 152; and VH-CDR3 comprising the amino acid sequence of SEQ ID NO: 153.

In some embodiments, the binding molecule, in particular the antibody or the antigen-binding fragment thereof, comprises VH-CDR1 comprising the amino acid sequence of SEQ ID NO: 161; VH-CDR2 comprising the amino acid sequence of SEQ ID NO: 162; and VH-CDR3 comprising the amino acid sequence of SEQ ID NO: 163. Thus, the invention also provides an amyloid-beta binding molecule, in particular an amyloid-beta antibody or antigen-binding fragment thereof, which comprises VH-CDR1 comprising the amino acid sequence of SEQ ID NO: 161; VH-CDR2 comprising the amino acid sequence of SEQ ID NO: 162; and VH-CDR3 comprising the amino acid sequence of SEQ ID NO: 163.

In some embodiments, the binding molecule, in particular the antibody or the antigen-binding fragment thereof, comprises VH-CDR1 comprising the amino acid sequence of SEQ ID NO: 171; VH-CDR2 comprising the amino acid sequence of SEQ ID NO: 172; and VH-CDR3 comprising the amino acid sequence of SEQ ID NO: 173. Thus, the invention also provides an amyloid-beta binding molecule, in particular an amyloid-beta antibody or antigen-binding fragment thereof, which comprises VH-CDR1 comprising the amino acid sequence of SEQ ID NO: 171; VH-CDR2 comprising the amino acid sequence of SEQ ID NO: 172; and VH-CDR3 comprising the amino acid sequence of SEQ ID NO: 173.

In some embodiments, the binding molecule, in particular the antibody or the antigen-binding fragment thereof, comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183. Thus, the invention also provides an amyloid-beta binding molecule, in particular an amyloid-beta antibody or antigen-binding fragment thereof, which comprises VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183.

In some embodiments, the binding molecule, in particular the antibody or the antigen-binding fragment thereof, comprises VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37. Thus, the invention also provides an amyloid-beta binding molecule, in particular an amyloid-beta antibody or antigen-binding fragment thereof, which comprises VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37.

In some embodiments, the binding molecule, in particular the antibody or the antigen-binding fragment thereof, comprises VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47. Thus, the invention also provides an amyloid-beta binding molecule, in particular an amyloid-beta antibody or antigen-binding fragment thereof, which comprises VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47.

In some embodiments, the binding molecule, in particular the antibody or the antigen-binding fragment thereof, comprises VL-CDR1 comprising the amino acid sequence of SEQ ID NO: 65; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VL-CDR1 comprising the amino acid sequence of SEQ ID NO: 65; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VL-CDR1 comprising the amino acid sequence of SEQ ID NO: 75; VL-CDR2 comprising the amino acid sequence of SEQ ID NO: 16; and VL-CDR3 comprising the amino acid sequence of SEQ ID NO: 17. Thus, the invention also provides an amyloid-beta binding molecule, in particular an amyloid-beta antibody or antigen-binding fragment thereof, which comprises VL-CDR1 comprising the amino acid sequence of SEQ ID NO: 75; VL-CDR2 comprising the amino acid sequence of SEQ ID NO: 16; and VL-CDR3 comprising the amino acid sequence of SEQ ID NO: 17.

In some embodiments, the binding molecule, in particular the antibody or the antigen-binding fragment thereof, comprises VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87. Thus, the invention also provides an amyloid-beta binding molecule, in particular an amyloid-beta antibody or antigen-binding fragment thereof, which comprises VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87.

In some embodiments, the binding molecule, in particular the antibody or the antigen-binding fragment thereof, comprises VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97. Thus, the invention also provides an amyloid-beta binding molecule, in particular an amyloid-beta antibody or antigen-binding fragment thereof, which comprises VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97.

In some embodiments, the binding molecule, in particular the antibody or the antigen-binding fragment thereof, comprises VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107. Thus, the invention also provides an amyloid-beta binding molecule, in particular an amyloid-beta antibody or antigen-binding fragment thereof, which comprises VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107.

In some embodiments, the binding molecule, in particular the antibody or the antigen-binding fragment thereof, comprises VL-CDR1 comprising the amino acid sequence of SEQ ID NO: 115; VL-CDR2 comprising the amino acid sequence of SEQ ID NO: 116; and VL-CDR3 comprising the amino acid sequence of SEQ ID NO: 117. Thus, the invention also provides an amyloid-beta binding molecule, in particular an amyloid-beta antibody or antigen-binding fragment thereof, which comprises VL-CDR1 comprising the amino acid sequence of SEQ ID NO: 115; VL-CDR2 comprising the amino acid sequence of SEQ ID NO: 116; and VL-CDR3 comprising the amino acid sequence of SEQ ID NO: 117.

In some embodiments, the binding molecule, in particular the antibody or the antigen-binding fragment thereof, comprises VL-CDR1 comprising the amino acid sequence of SEQ ID NO: 125; VL-CDR2 comprising the amino acid sequence of SEQ ID NO: 126; and VL-CDR3 comprising the amino acid sequence of SEQ ID NO: 127. Thus, the invention also provides an amyloid-beta binding molecule, in particular an amyloid-beta antibody or antigen-binding fragment thereof, which comprises VL-CDR1 comprising the amino acid sequence of SEQ ID NO: 125; VL-CDR2 comprising the amino acid sequence of SEQ ID NO: 126; and VL-CDR3 comprising the amino acid sequence of SEQ ID NO: 127.

In some embodiments, the binding molecule, in particular the antibody or the antigen-binding fragment thereof, comprises VL-CDR1 comprising the amino acid sequence of SEQ ID NO: 135; VL-CDR2 comprising the amino acid sequence of SEQ ID NO: 16; and VL-CDR3 comprising the amino acid sequence of SEQ ID NO: 137. Thus, the invention also provides an amyloid-beta binding molecule, in particular an amyloid-beta antibody or antigen-binding fragment thereof, which comprises VL-CDR1 comprising the amino acid sequence of SEQ ID NO: 135; VL-CDR2 comprising the amino acid sequence of SEQ ID NO: 16; and VL-CDR3 comprising the amino acid sequence of SEQ ID NO: 137.

In some embodiments, the binding molecule, in particular the antibody or the antigen-binding fragment thereof, comprises VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 137. Thus, the invention also provides an amyloid-beta binding molecule, in particular an amyloid-beta antibody or antigen-binding fragment thereof, which comprises VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 137.

In some embodiments, the binding molecule, in particular the antibody or the antigen-binding fragment thereof, comprises VL-CDR1 comprising the amino acid sequence of SEQ ID NO: 155; VL-CDR2 comprising the amino acid sequence of SEQ ID NO: 156; and VL-CDR3 comprising the amino acid sequence of SEQ ID NO: 157. Thus, the invention also provides an amyloid-beta binding molecule, in particular an amyloid-beta antibody or antigen-binding fragment thereof, which comprises VL-CDR1 comprising the amino acid sequence of SEQ ID NO: 155; VL-CDR2 comprising the amino acid sequence of SEQ ID NO: 156; and VL-CDR3 comprising the amino acid sequence of SEQ ID NO: 157.

In some embodiments, the binding molecule, in particular the antibody or the antigen-binding fragment thereof, comprises VL-CDR1 comprising the amino acid sequence of SEQ ID NO: 175; VL-CDR2 comprising the amino acid sequence of SEQ ID NO: 156; and VL-CDR3 comprising the amino acid sequence of SEQ ID NO: 157. Thus, the invention also provides an amyloid-beta binding molecule, in particular an amyloid-beta antibody or antigen-binding fragment thereof, which comprises VL-CDR1 comprising the amino acid sequence of SEQ ID NO: 175; VL-CDR2 comprising the amino acid sequence of SEQ ID NO: 156; and VL-CDR3 comprising the amino acid sequence of SEQ ID NO: 157.

In some embodiments, the binding molecule, in particular the antibody or the antigen-binding fragment thereof, comprises VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 187. Thus, the invention also provides an amyloid-beta binding molecule, in particular an amyloid-beta antibody or antigen-binding fragment thereof, which comprises VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 187.

In some embodiments, the binding molecule, in particular the antibody or the antigen-binding fragment thereof, comprises VL-CDR1 comprising the amino acid sequence of SEQ ID NO: 195; VL-CDR2 comprising the amino acid sequence of SEQ ID NO: 196; and VL-CDR3 comprising the amino acid sequence of SEQ ID NO: 197. Thus, the invention also provides an amyloid-beta binding molecule, in particular an amyloid-beta antibody or antigen-binding fragment thereof, which comprises VL-CDR1 comprising the amino acid sequence of SEQ ID NO: 195; VL-CDR2 comprising the amino acid sequence of SEQ ID NO:196; and VL-CDR3 comprising the amino acid sequence of SEQ ID NO: 197.

In some embodiments, the binding molecule, in particular the antibody or the antigen-binding fragment thereof, comprises a Heavy Chain (HC) comprising the amino sequence of SEQ ID NO: 20. In some embodiments, the binding molecule, in particular the antibody or the antigen-binding fragment thereof, comprises a Heavy Chain (HC) comprising the amino sequence of SEQ ID NO: 22. In some embodiments, the binding molecule, in particular the antibody or the antigen-binding fragment thereof, comprises a Heavy Chain (HC) comprising the amino sequence of SEQ ID NO: 24. In some embodiments, the binding molecule, in particular the antibody or the antigen-binding fragment thereof, comprises a Heavy Chain (HC) comprising the amino sequence of SEQ ID NO: 27. In some embodiments, the binding molecule, in particular the antibody or the antigen-binding fragment thereof, comprises a Heavy Chain (HC) comprising the amino sequence of SEQ ID NO: 38. In some embodiments, the binding molecule, in particular the antibody or the antigen-binding fragment thereof, comprises a Heavy Chain (HC) comprising the amino sequence of SEQ ID NO: 48. In some embodiments, the binding molecule, in particular the antibody or the antigen-binding fragment thereof, comprises a Heavy Chain (HC) comprising the amino sequence of SEQ ID NO: 50. In some embodiments, the binding molecule, in particular the antibody or the antigen-binding fragment thereof, comprises a Heavy Chain (HC) comprising the amino sequence of SEQ ID NO: 56. In some embodiments, the binding molecule, in particular the antibody or the antigen-binding fragment thereof, comprises a Heavy Chain (HC) comprising the amino sequence of SEQ ID NO: 58.

In some embodiments, the binding molecule, in particular the antibody or the antigen-binding fragment thereof, comprises a Light Chain (LC) comprising the amino sequence of SEQ ID NO: 21. In some embodiments, the binding molecule, in particular the antibody, or the antigen-binding fragment thereof, comprises a Light Chain (LC) comprising the amino sequence of SEQ ID NO: 23. In some embodiments, the binding molecule, in particular the antibody or the antigen-binding fragment thereof, comprises a Light Chain (LC) comprising the amino sequence of SEQ ID NO: 25. In some embodiments, the binding molecule, in particular the antibody or the antigen-binding fragment thereof, comprises a Light Chain (LC) comprising the amino sequence of SEQ ID NO: 28. In some embodiments, the binding molecule, in particular the antibody or the antigen-binding fragment thereof, comprises a Light Chain (LC) comprising the amino sequence of SEQ ID NO: 39. In some embodiments, the binding molecule, in particular the antibody or the antigen-binding fragment thereof, comprises a Light Chain (LC) comprising the amino sequence of SEQ ID NO: 49. In some embodiments, the binding molecule, in particular the antibody or the antigen-binding fragment thereof, comprises a Light Chain (LC) comprising the amino sequence of SEQ ID NO: 51. In some embodiments, the binding molecule, in particular the antibody or the antigen-binding fragment thereof, comprises a Light Chain (LC) comprising the amino sequence of SEQ ID NO: 57. In some embodiments, the binding molecule, in particular the antibody or the antigen-binding fragment thereof, comprises a Light Chain (LC) comprising the amino sequence of SEQ ID NO: 59.

In some embodiments, the binding molecule, in particular the antibody or the antigen-binding fragment thereof, comprises a Heavy Chain (HC) comprising the amino sequence of SEQ ID NO: 20 and comprises a Light Chain (LC) comprising the amino sequence of SEQ ID NO: 21. In some embodiments, the binding molecule, in particular the antibody, or the antigen-binding fragment thereof, comprises a Heavy Chain (HC) comprising the amino sequence of SEQ ID NO: 22 and comprises a Light Chain (LC) comprising the amino sequence of SEQ ID NO: 23. In some embodiments, the binding molecule, in particular the antibody or the antigen-binding fragment thereof, comprises a Heavy Chain (HC) comprising the amino sequence of SEQ ID NO: 24 and comprises a Light Chain (LC) comprising the amino sequence of SEQ ID NO: 25. In some embodiments, the binding molecule, in particular the antibody or the antigen-binding fragment thereof, comprises a Heavy Chain (HC) comprising the amino sequence of SEQ ID NO: 27 and comprises a Light Chain (LC) comprising the amino sequence of SEQ ID NO: 28. In some embodiments, the binding molecule, in particular the antibody or the antigen-binding fragment thereof, comprises a Heavy Chain (HC) comprising the amino sequence of SEQ ID NO: 38 and comprises a Light Chain (LC) comprising the amino sequence of SEQ ID NO: 39. In some embodiments, the binding molecule, in particular the antibody or the antigen-binding fragment thereof, comprises a Heavy Chain (HC) comprising the amino sequence of SEQ ID NO: 48 and comprises a Light Chain (LC) comprising the amino sequence of SEQ ID NO: 49. In some embodiments, the binding molecule, in particular the antibody or the antigen-binding fragment thereof, comprises a Heavy Chain (HC) comprising the amino sequence of SEQ ID NO: 50 and comprises a Light Chain (LC) comprising the amino sequence of SEQ ID NO: 51. In some embodiments, the binding molecule, in particular the antibody or the antigen-binding fragment thereof, comprises a Heavy Chain (HC) comprising the amino sequence of SEQ ID NO: 56 and comprises a Light Chain (LC) comprising the amino sequence of SEQ ID NO: 57. In some embodiments, the binding molecule, in particular the antibody or the antigen-binding fragment thereof, comprises a Heavy Chain (HC) comprising the amino sequence of SEQ ID NO: 58 and comprises a Light Chain (LC) comprising the amino sequence of SEQ ID NO: 59.

The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 13; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 17. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 31; VH-CDR2 comprising the amino acid sequence of SEQ ID NO: 32; and VH-CDR3 comprising the amino acid sequence of SEQ ID NO: 33; VL-CDR1 comprising the amino acid sequence of SEQ ID NO: 35; VL-CDR2 comprising the amino acid sequence of SEQ ID NO: 36; and VL-CDR3 comprising the amino acid sequence of SEQ ID NO: 37. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 41; VH-CDR2 comprising the amino acid sequence of SEQ ID NO: 42; and VH-CDR3 comprising the amino acid sequence of SEQ ID NO: 43; VL-CDR1 comprising the amino acid sequence of SEQ ID NO: 45; VL-CDR2 comprising the amino acid sequence of SEQ ID NO: 46; and VL-CDR3 comprising the amino acid sequence of SEQ ID NO: 47. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 63; VL-CDR1 comprising the amino acid sequence of SEQ ID NO: 65; VL-CDR2 comprising the amino acid sequence of SEQ ID NO: 16; and VL-CDR3 comprising the amino acid sequence of SEQ ID NO: 17. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 11; VH-CDR2 comprising the amino acid sequence of SEQ ID NO: 12; and VH-CDR3 comprising the amino acid sequence of SEQ ID NO: 73; VL-CDR1 comprising the amino acid sequence of SEQ ID NO: 75; VL-CDR2 comprising the amino acid sequence of SEQ ID NO: 16; and VL-CDR3 comprising the amino acid sequence of SEQ ID NO: 17. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 61; VH-CDR2 comprising the amino acid sequence of SEQ ID NO: 62; and VH-CDR3 comprising the amino acid sequence of SEQ ID NO: 83; VL-CDR1 comprising the amino acid sequence of SEQ ID NO: 85; VL-CDR2 comprising the amino acid sequence of SEQ ID NO: 16; and VL-CDR3 comprising the amino acid sequence of SEQ ID NO: 87. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 91; VH-CDR2 comprising the amino acid sequence of SEQ ID NO: 92; and VH-CDR3 comprising the amino acid sequence of SEQ ID NO: 93; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 97. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 101; VH-CDR2 comprising the amino acid sequence of SEQ ID NO: 102; and VH-CDR3 comprising the amino acid sequence of SEQ ID NO: 103; VL-CDR1 comprising the amino acid sequence of SEQ ID NO: 105; VL-CDR2 comprising the amino acid sequence of SEQ ID NO: 106; and VL-CDR3 comprising the amino acid sequence of SEQ ID NO: 107. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 111; VH-CDR2 comprising the amino acid sequence of SEQ ID NO: 112; and VH-CDR3 comprising the amino acid sequence of SEQ ID NO: 113; VL-CDR1 comprising the amino acid sequence of SEQ ID NO: 115; VL-CDR2 comprising the amino acid sequence of SEQ ID NO: 116; and VL-CDR3 comprising the amino acid sequence of SEQ ID NO: 117. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 121; VH-CDR2 comprising the amino acid sequence of SEQ ID NO: 122; and VH-CDR3 comprising the amino acid sequence of SEQ ID NO: 123; VL-CDR1 comprising the amino acid sequence of SEQ ID NO: 125; VL-CDR2 comprising the amino acid sequence of SEQ ID NO: 126; and VL-CDR3 comprising the amino acid sequence of SEQ ID NO: 127. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 131; VH-CDR2 comprising the amino acid sequence of SEQ ID NO: 132; and VH-CDR3 comprising the amino acid sequence of SEQ ID NO: 133; VL-CDR1 comprising the amino acid sequence of SEQ ID NO: 135; VL-CDR2 comprising the amino acid sequence of SEQ ID NO: 16; and VL-CDR3 comprising the amino acid sequence of SEQ ID NO: 137. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 141; VH-CDR2 comprising the amino acid sequence of SEQ ID NO: 142; and VH-CDR3 comprising the amino acid sequence of SEQ ID NO: 133; VL-CDR1 comprising the amino acid sequence of SEQ ID NO: 15; VL-CDR2 comprising the amino acid sequence of SEQ ID NO: 16; and VL-CDR3 comprising the amino acid sequence of SEQ ID NO: 137. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 151; VH-CDR2 comprising the amino acid sequence of SEQ ID NO: 152; and VH-CDR3 comprising the amino acid sequence of SEQ ID NO: 153; VL-CDR1 comprising the amino acid sequence of SEQ ID NO: 155; VL-CDR2 comprising the amino acid sequence of SEQ ID NO: 156; and VL-CDR3 comprising the amino acid sequence of SEQ ID NO: 157. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 161; VH-CDR2 comprising the amino acid sequence of SEQ ID NO: 162; and VH-CDR3 comprising the amino acid sequence of SEQ ID NO: 163; VL-CDR1 comprising the amino acid sequence of SEQ ID NO: 165; VL-CDR2 comprising the amino acid sequence of SEQ ID NO: 166; and VL-CDR3 comprising the amino acid sequence of SEQ ID NO: 167. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 171; VH-CDR2 comprising the amino acid sequence of SEQ ID NO: 172; and VH-CDR3 comprising the amino acid sequence of SEQ ID NO: 173; VL-CDR1 comprising the amino acid sequence of SEQ ID NO: 175; VL-CDR2 comprising the amino acid sequence of SEQ ID NO: 156; and VL-CDR3 comprising the amino acid sequence of SEQ ID NO: 157. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 95; VL-CDR2 comprising the amino acid sequence of SEQ ID NO: 96; and VL-CDR3 comprising the amino acid sequence of SEQ ID NO: 187. The binding molecules, antibodies or antigen-binding fragments thereof, may additionally or alternatively comprise amino acid changes in the CDR sequences comprised therein and as compared to the CDR sequences provided in VH-CDR1 comprising the amino acid sequence of SEQ ID NO: 181; VH-CDR2 comprising the amino acid sequence of SEQ ID NO: 182; and VH-CDR3 comprising the amino acid sequence of SEQ ID NO: 183; VL-CDR1 comprising the amino acid sequence of SEQ ID NO: 195; VL-CDR2 comprising the amino acid sequence of SEQ ID NO: 196; and VL-CDR3 comprising the amino acid sequence of SEQ ID NO: 197. In particular, the CDR sequences comprised in the binding molecules, antibodies or antigen-binding fragments thereof, may comprise one, two or three amino acid changes with respect to the CDR sequences provided herein. An “amino acid change” may relate to a mutation, deletion or addition of an amino acid. It may also relate to the chemical alteration of an amino acid, such as for example the formation of a non-natural amino acid.

Amino acid sequence variants of the binding molecules, in particular antibodies or antigen-binding fragments thereof provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In certain embodiments, antibody variants having one or more amino acid substitutions are provided. Sites of interest for substitutional mutagenesis include the CDRs and framework regions (FRs). Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding or improved selectivity.

TABLE A

| Original | | Preferred |
|---|---|---|
| Residue | Exemplary Substitutions | Substitutions |
| Ala (A) | Val; Leu; Ile | Val |
| Arg (R) | Lys; Gin; Asn | Lys |
| Asn (N) | Gin; His; Asp, Lys; Arg | Gin |
| Asp (D) | Glu; Asn | Glu |
| Cys (C) | Ser; Ala | Ser |
| Gin (Q) | Asn; Glu | Asn |
| Glu (E) | Asp; Gin | Asp |
| Gly (G) | Ala | Ala |
| His (H) | Asn; Gin; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gin; Asn | Arg |
| Met (M) | Leu; Phe; Ile | Leu |
| Phe (F) | Trp; Leu; Val; He; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant VH or VL being tested for binding affinity. Affinity maturation by constructing and reselecting from secondary libraries has been described, e.g., in Hoogenboom et al., in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may be outside of CDR “hotspots” or SDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244: 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex is used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme or other label (e.g. HRP or AP) or a polypeptide which increases the serum half-life of the antibody.

Within the scope of the present invention, amyloid-beta may have the sequence of abeta1-43 (SEQ ID NO: 3), abeta1-42 (SEQ ID NO: 1), abeta1-41 (SEQ ID NO: 4), abeta1-40 (SEQ ID NO: 5), or abeta1-39 (SEQ ID NO: 6) or fragment thereof.

The term “amyloid-beta peptides or species” refers to amyloid-beta produced by cleavage of amyloid precursor protein (APP) by beta- and gamma-secretase activity. Thus, “amyloid-beta peptides or species” refers to amyloid-beta produced by the amyloidogenic pathway. However, the term is intended to also encompass synthetically produced peptides. Those peptides will have the same amino acid sequence but are not produced by the process that occurs in vivo. Methods of peptide synthesis are well known in the art and commercially available. The amyloid-beta peptides or species may mainly comprise amyloid-beta 1-42 (Abeta1-42) or 1-43 (Abeta1-43) and (shorter) fragments thereof. The amyloid-beta peptides or species may incorporate post-translational modifications (PTM), including, but not limited to, phosphorylation (such as phosphorylation of serine at amino acid residue 8 of amyloid-beta (pS8-Abeta)), glycosylation, oligomerization and/or proteolytic cleavage. A synthetic process may be used in order to add some PTMs. This may involve natural components, such as kinases, used in an in vitro environment for example. The amyloid-beta peptides or species may include (but are not limited to) the following amyloid-beta peptides: Abeta1-38, Abeta1-39, Abeta1-40, pS8-Abeta1-40, Abeta1-42, pS8-Abeta1-42, and Abeta1-43 and (shorter) fragments thereof. Abeta peptide fragments will include the epitope recognized by the binding molecule. The amyloid-beta peptides or species may adopt different conformational states, such as monomers, oligomers or fibrillar structures.

The amyloid precursor protein (APP), an integral membrane protein, undergoes proteolytic processing to generate multiple peptide fragments some of which are released and are present in sample of body fluids (which may be referred to herein as “biofluids”). The most prominent APP peptide fragments found in biofluids include peptides in the Abeta family (Abeta1-43, Abeta1-42, Abeta1-41, Abeta1-40, and Abeta1-39) released by beta- and gamma-secretase activities of the amyloidogenic pathway. Another cleavage product of APP that is found in biofluids is the soluble APP alpha (sAPPalpha), released by the alpha-secretases of the non-amyloidogenic pathway. The binding molecules of the invention with no cross reactivity to sAPPalpha when used in a combination, such as when paired with a second Abeta antibody in a sandwich assay, advantageously do not detect sAPPalpha.

Thus, a binding molecule, in particular an antibody or antigen-binding fragment thereof, of the invention may selectively bind or capture any amyloid-beta peptides or species in solution, independently of the conformational state of the amyloid-beta peptides or species, with no cross-reactivity to soluble APP alpha. Some binding molecules, in particular an antibody or antigen-binding fragment thereof, of the invention may selectively bind or capture any amyloid-beta peptides or species in solution, independently of the conformational state of the amyloid-beta peptides or species, with low cross-reactivity to soluble APP alpha. Some binding molecules of the invention may show higher cross-reactivity with sAPPalpha. The binding molecules demonstrating no, low or high cross-reactivity to soluble APP alpha may be combined, for use in sandwich immunoassays, with the proviso that one of the binding molecules of the inventions is employed demonstrating no cross reactivity to sAPPalpha. In this event, the assay displays an overall pair specific signal for Abeta and no signal for sAPPalpha. Preferably, the solution is a body fluid, as described herein, or a buffer solution. More preferably, the solution is a body fluid. Body fluid sample is defined as saliva, urine, nasal secretion, blood, brain and/or CSF, brain and/or ISF, more particularly a blood, brain and/or CSF or brain and/or ISF. Blood samples may be whole blood, serum or plasma samples for example, but are preferably plasma samples.

Binding molecules, in particular antibodies or antigen-binding fragments thereof, of the invention bind to an epitope within the amino acid sequence of SEQ ID NO: 1. SEQ ID NO: 1 is the amino acid sequence of human Abeta 1-42. It will be appreciated that equivalent binding regions exist in non-human Abeta. Thus, for example, the mouse Abeta 1-42 amino acid sequence is 93% (39/42 residues) identical with the human sequence. The invention encompasses binding molecules, in particular antibodies or antigen-binding fragments thereof, that bind to equivalent regions/peptides to those specified above with reference to SEQ ID NO: 1 in non-human Abeta, especially murine Abeta. The Abeta 1-42 amino acid sequence is identical between mouse and rat. In certain embodiments, binding molecules, in particular antibodies or antigen-binding fragments thereof, of the invention bind to an epitope within amino acids residues 1-8 (SEQ ID NO: 2) of SEQ ID NO: 1. In certain embodiments, binding molecules, in particular antibodies or antigen-binding fragments, of the invention bind to an epitope within amino acids residues 1-8 (SEQ ID NO: 2), 1-5 (SEQ ID NO: 7), 17-23 (SEQ ID NO: 9), 22-35 (SEQ ID NO: 29) or 26-34 (SEQ ID NO: 8) of SEQ ID NO: 1. In some embodiments, amino acids 1 and 2 of Abeta 1-42 are essential to antibody binding, in particular for antibody ACI-24-41F12-Ab2 and related (e.g. by common CDR and/or VH/VL sequences) as described herein (see SEQ ID Nos 10-25 and associated embodiments). Without wishing to be bound by theory, this antibody is thought to bind to an epitope that is inaccessible in soluble APPalpha. Soluble APPalpha includes amino acids 1-5 and 1-8 of Abeta1-42 but this antibody does not bind to sAPPalpha with high affinity.

In certain embodiments, a binding molecule, in particular an antibody or antigen-binding fragment thereof, of the invention and as provided herein has a dissociation constant (KD) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M), in particular with respect to binding abeta1-42. In some embodiments, a binding molecule, in particular an antibody or antigen-binding fragment thereof, that binds human amyloid-beta is provided, wherein the binding molecule, in particular the antibody or antigen-binding fragment thereof, binds abeta with a KD of less than 100 nM, less than 10 nM, less than 1 nM, less than 200 pM, or less than 100 pM. Within the present invention, it is preferred that the KD is determined using ELISA, such as described in Example 3 provided herein.

The terms “binding molecule”, “amyloid-beta antibody”, “anti-amyloid-beta antibody”, “abeta antibody” or simply “antibody” as used herein refer to a binding molecule, more particularly an antibody that is capable of binding Aβ monomer and/or soluble Aβ oligomers with sufficient affinity such that the binding molecule, antibody or antigen-binding fragment thereof, is useful as a diagnostic and/or therapeutic agent in targeting amyloid-beta. In one embodiment, the extent of binding of a binding molecule, in particular an amyloid-beta antibody or an antigen-binding fragment thereof, of the invention to an unrelated, non-amyloid-beta protein is less than about 10% of the binding of the antibody to amyloid-beta as measured, e.g., by a radioimmunoassay (RIA).

In general, the term “antibody” is used herein in the broadest sense and encompasses various antibody structures, including but not limited to monoclonal antibodies, polyclonal antibodies, multispecific antibodies (e.g., bispecific, biparatopic antibodies), fully-human antibodies and antibody fragments so long as they exhibit the desired antigen-binding activity. Antibodies within the present invention may also be chimeric antibodies, recombinant antibodies, antigen-binding fragments of recombinant antibodies, humanized antibodies or antibodies displayed upon the surface of a phage or displayed upon the surface of a chimeric antigen receptor (CAR) T-cell.

An “antigen-binding fragment” of an antibody refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

The term “monoclonal antibody” as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modified “monoclonal” indicates the character of the antibody as being amongst a substantially homogeneous population of antibodies and is not to be construed as requiring production of the antibody by any particular method. The monoclonal antibodies to be used in accordance with the present invention may be made by the hybridoma method described by Kohler, Nature 256 (1975), 495.

Accordingly, in context of the present invention, the term “antibody” relates to full immunoglobulin molecules as well as to parts of such immunoglobulin molecules (i.e., “antigen-binding fragment thereof”). Furthermore, the term relates, as discussed above, to modified and/or altered antibody molecules. The term also relates to recombinantly or synthetically generated/synthesized antibodies. The term also relates to intact antibodies as well as to antibody fragments thereof, like, separated light and heavy chains, Fab, Fv, Fab′, Fab′-SH, F(ab′)2. The term antibody also comprises but is not limited to fully human antibodies, chimeric antibodies, humanized antibodies, CDR-grafted antibodies and antibody constructs, like single chain Fvs (scFv) or antibody-fusion proteins.

The term “CDR” as employed herein relates to “complementary determining region”, which is well known in the art. The CDRs are parts of immunoglobulins that determine the specificity of said molecules and make contact with a specific ligand. The CDRs are the most variable part of the molecule and contribute to the diversity of these molecules. There are three CDR regions CDR1, CDR2 and CDR3 in each V domain. VH-CDR, or CDR-H depicts a CDR region of a Variable Heavy Chain and VL-CDR or CDR-L relates to a CDR region of a Variable Light Chain. VH means the Variable Heavy Chain and VL means the Variable Light Chain. The CDR regions of an Ig-derived region may be determined as described in Kabat “Sequences of Proteins of Immunological Interest”, 5th edit. NIH Publication no. 91-3242 U.S. Department of Health and Human Services (1991); Chothia J., Mol. Biol. 196 (1987), 901-917 or Chothia, Nature 342 (1989), 877-883. CDR sequences provided herein are defined according to Kabat. However, it will be understood by the skilled person that the invention is intended to encompass binding molecules in which the CDR sequences are defined according to any useful identification/numbering scheme. For example, Chothia (Canonical structures for the hypervariable regions of immunoglobulins. Chothia C, Lesk A M. J Mol Biol. 1987 Aug. 20; 196(4):901-17), IMGT (IMGT, the international ImMunoGeneTics database. Giudicelli V, Chaume D, Bodmer J, Müller W, Busin C, Marsh S, Bontrop R, Marc L, Malik A, Lefranc M P. Nucleic Acids Res. 1997 Jan. 1; 25(1):206-11 and Unique database numbering system for immunogenetic analysis. Lefranc M P. Immunol Today. 1997 November; 18(11):509), MacCallum (MacCallum R M, Martin A C, Thornton J M, J Mol Biol. 1996 Oct. 11; 262(5):732-45) and Martin (Abhinandan K R, Martin A C R. Analysis and improvements to Kabat and structurally correct numbering of antibody variable domains. Mol Immunol. (2008) 45:3832-9. 10.1016/j.molimm.2008.05.022) numbering schemes may be adopted in order to define the CDRs.

An “Fc” region contains two heavy chain fragments comprising the CH2 and CH3 domains of an antibody. The two heavy chain fragments are held together by two or more disulfide bonds and by hydrophobic interactions of the CH3 domains.

The “Fv region” comprises the variable regions from both the heavy and light chains, but lacks the constant regions.

An “antibody that binds to an epitope” within a defined region of a protein is an antibody that requires the presence of one or more of the amino acids within that region for binding to the protein.

In certain embodiments, one or more amino acid modifications may be introduced into the Fc region of an antibody provided herein, thereby generating an Fc region variant. The Fc region variant may comprise a murine Fc region sequence, an antibody isotype or class (e.g. IgG1, IgG2a, IgG2b or IgG2c) comprising an amino acid modification (e.g. substitution) at one or more amino acid positions. The Fc region variant may comprise a human Fc region sequence (an antibody isotype or class, e.g., a human IgM, IgG1, IgG2, IgG3 or IgG4 Fc region) comprising an amino acid modification (e.g. a substitution) at one or more amino acid positions. An antibody isotype or class refers to the type of constant domain, region, or sequence contained within its heavy chain. There are five major classes of antibodies: IgA, IgD, IgE, IgG, and IgM, and several of these may be further divided into subclasses or isotypes, e.g., IgA1, IgA2, IgG1, IgG2, IgG2a (for murine only), IgG2b (for murine only), IgG2c (for murine only), IgG3, and IgG4. The heavy chain constant domains that correspond to the different classes of immunoglobulins are called α, δ, ε, γ, and μ, respectively. The antibody light chain constant domain, region, or sequence can be one of two major classes; kappa (κ) or lambda (λ). When the light chain class is not specified it is assumed to be the more common κ light chain. In some embodiments, the antibody or the antigen-binding fragment thereof, of the invention comprises murine IgG2a or chimeric (mouse/human) IgG1 isotypes.

In some embodiments, an antibody or an antigen-binding fragment thereof, of the invention may be produced recombinantly. In some embodiments, a(n isolated) nucleic acid is provided, wherein the (isolated) nucleic acid encodes a binding molecule, in particular an antibody or an antigen-binding fragment thereof of the invention. The term “nucleic acid” as used herein refers to a nucleic acid molecule. In some embodiments, a host cell or a cell-free expression system is provided, wherein the host cell or the cell-free expression system comprises a(n isolated) nucleic acid that encodes a binding molecule, in particular an antibody or an antigen-binding fragment thereof of the invention. In some embodiments, a host cell is transfected with a vector comprising a(n isolated) nucleic acid encoding a binding molecule, in particular an antibody or an antigen-binding fragment thereof, of the invention. In some embodiments, a host cell is transfected with a vector comprising a(n isolated) nucleic acid encoding the Heavy Chain (HC) and the Light Chain (LC) of a binding molecule, in particular an antibody or an antigen-binding fragment thereof, of the invention. In some embodiments, a host cell is transfected with a) a first vector comprising a(n isolated) nucleic acid encoding the Heavy Chain (HC) of a binding molecule, in particular an antibody or an antigen-binding fragment thereof, of the invention, and with b) a second vector comprising a(n isolated) nucleic acid encoding the Light Chain (LC) of a binding molecule, in particular an antibody or an antigen-binding fragment thereof, of the invention. In some embodiments, the host cell can be, but is not limited to, a Chinese Hamster Ovary (CHO) cell. Suitable host cells may be prokaryote, yeast, or higher eukaryote cells, specifically mammalian cells. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen. Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); mouse myeloma cells SP2/0-AG14 (ATCC CRL 1581; ATCC CRL 8287) or NSO (HPA culture collections no. 85110503); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), as well as DSM's PERC-6 cell line. Expression vectors suitable for use in each of these host cells are also generally known in the art. The term “host cell” generally refers to a cultured cell line. Accordingly, whole human beings into which an expression vector encoding an antigen binding polypeptide according to the invention has been introduced are explicitly excluded from the definition of a “host cell”. Cell-free expression systems may be based on use of cell lysates or extracts, such as CHO cell lysates (Stech, M., Nikolaeva, O., Thoring, L. et al. Cell-free synthesis of functional antibodies using a coupled in vitro transcription-translation system based on CHO cell lysates. Sci Rep 7, 12030 (2017)).

In some embodiments, a(n isolated) nucleic acid is provided, wherein the (isolated) nucleic acid encodes a binding molecule, in particular an antibody or an antigen-binding fragment thereof, of the invention as provided herein. In some embodiments, a host cell is provided, wherein the host cell comprises a(n isolated) nucleic acid that encodes a binding molecule, in particular an antibody or an antigen-binding fragment thereof, of the invention as provided herein. In some embodiments, a method of producing a binding molecule, in particular an antibody or an antigen-binding fragment thereof, of the invention as provided herein, comprising culturing the host cell or a cell-free expression system under conditions suitable for producing the binding molecule, in particular the antibody or the antigen-binding fragment thereof. In some embodiments, a method for producing a(n isolated) binding molecule, in particular an antibody or an antigen-binding fragment thereof, of the invention as provided herein, comprising the steps of: a) culturing the host cell or the cell-free expression system under conditions suitable for producing the binding molecule, in particular antibody or antigen-binding fragment thereof, and b) recovering the binding molecule, in particular antibody or antigen-binding fragment thereof. Recovery of the binding molecule, in particular antibody or antigen-binding fragment thereof may be performed by any suitable means as would be understood by one skilled in the art. Recovery may comprise purification to a desired level of purity. Recovery may comprise isolation of the antibody from the culture. Recovery may involve use of chromatography for example. Such production methods may be performed at scale and thus may enable large-scale production of the binding molecules of the invention. A binding molecule produced according to this method is also provided, which may be considered an isolated binding molecule.

As used herein, the term “isolated” means that the molecule, e.g. the nucleic acid or antibody, has been separated and/or recovered from its natural environment. Within the present invention, the molecule is preferably chemically synthesized, or synthesized in a cellular system different from the cell from which it naturally originates, and is thus “isolated” from its naturally associated components. The molecule may be isolated from its natural environment by e.g. purification or produced by means of a technical process (including but not limited to gene synthesis, polymerase chain reaction (PCR), vector purification and protein (antibody) purification). Such molecules may be, in particular, a nucleic acid, such as DNA-, RNA-, or cDNA-sequence, or a peptide, antibody or protein.

The present invention is not limited to an isolated antibody or an isolated nucleic acid in accordance with the above definition, but also relates to an antibody or nucleic acid as such irrespective of its origin.

The same applies to peptides, nucleic acids, DNA, RNA and/or cDNA sequences provided by the present invention, which are encompassed in isolated form, as defined above, or in any other form. Different techniques known in the art, including (but not limited to) SDS-polyacrylamide gel electrophoresis (SDS-PAGE), column chromatography, filtration, isoelectrofocusing, dialysis, recrystallization, ultrafiltration, salting-out, solvent precipitation, solvent extraction, distillation and immunoprecipitation, can be used to purify, isolate or separate the binding molecules, in particular antibodies or antigen-binding fragments or derivatives thereof, of the present invention from inside the cells or from outside the cells (e.g. medium).

In some embodiments, a(n isolated) nucleic acid is provided, wherein the (isolated) nucleic acid comprises SEQ ID NO: 18 encoding an abeta antibody or antigen-binding fragment thereof. In some embodiments, a(n isolated) nucleic acid is provided, wherein the (isolated) nucleic acid comprises SEQ ID NO: 19 encoding an abeta antibody or antigen-binding fragment thereof. In some embodiments, a(n isolated) nucleic acid is provided, wherein the (isolated) nucleic acid comprises SEQ ID NO: 52 encoding an abeta antibody or antigen-binding fragment thereof. In some embodiments, a(n isolated) nucleic acid is provided, wherein the (isolated) nucleic acid comprises SEQ ID NO: 53 encoding an abeta antibody or antigen-binding fragment thereof. In some embodiments, a(n isolated) nucleic acid is provided, wherein the (isolated) nucleic acid comprises SEQ ID NO: 54 encoding an abeta antibody or antigen-binding fragment thereof. In some embodiments, a(n isolated) nucleic acid is provided, wherein the (isolated) nucleic acid comprises SEQ ID NO: 55 encoding an abeta antibody or antigen-binding fragment thereof. In some embodiments, a(n isolated) nucleic acid is provided, wherein the (isolated) nucleic acid comprises SEQ ID NO: 68 encoding an abeta antibody or antigen-binding fragment thereof. In some embodiments, a(n isolated) nucleic acid is provided, wherein the (isolated) nucleic acid comprises SEQ ID NO: 69 encoding an abeta antibody or antigen-binding fragment thereof. In some embodiments, a(n isolated) nucleic acid is provided, wherein the (isolated) nucleic acid comprises SEQ ID NO: 78 encoding an abeta antibody or antigen-binding fragment thereof. In some embodiments, a(n isolated) nucleic acid is provided, wherein the (isolated) nucleic acid comprises SEQ ID NO: 79 encoding an abeta antibody or antigen-binding fragment thereof. In some embodiments, a(n isolated) nucleic acid is provided, wherein the (isolated) nucleic acid comprises SEQ ID NO: 88 encoding an abeta antibody or antigen-binding fragment thereof. In some embodiments, a(n isolated) nucleic acid is provided, wherein the (isolated) nucleic acid comprises SEQ ID NO: 89 encoding an abeta antibody or antigen-binding fragment thereof. In some embodiments, a(n isolated) nucleic acid is provided, wherein the (isolated) nucleic acid comprises SEQ ID NO: 98 encoding an abeta antibody or antigen-binding fragment thereof. In some embodiments, a(n isolated) nucleic acid is provided, wherein the (isolated) nucleic acid comprises SEQ ID NO: 99 encoding an abeta antibody or antigen-binding fragment thereof. In some embodiments, a(n isolated) nucleic acid is provided, wherein the (isolated) nucleic acid comprises SEQ ID NO: 108 encoding an abeta antibody or antigen-binding fragment thereof. In some embodiments, a(n isolated) nucleic acid is provided, wherein the (isolated) nucleic acid comprises SEQ ID NO: 109 encoding an abeta antibody or antigen-binding fragment thereof. In some embodiments, a(n isolated) nucleic acid is provided, wherein the (isolated) nucleic acid comprises SEQ ID NO: 118 encoding an abeta antibody or antigen-binding fragment thereof. In some embodiments, a(n isolated) nucleic acid is provided, wherein the (isolated) nucleic acid comprises SEQ ID NO: 119 encoding an abeta antibody or antigen-binding fragment thereof. In some embodiments, a(n isolated) nucleic acid is provided, wherein the (isolated) nucleic acid comprises SEQ ID NO: 128 encoding an abeta antibody or antigen-binding fragment thereof. In some embodiments, a(n isolated) nucleic acid is provided, wherein the (isolated) nucleic acid comprises SEQ ID NO: 129 encoding an abeta antibody or antigen-binding fragment thereof. In some embodiments, a(n isolated) nucleic acid is provided, wherein the (isolated) nucleic acid comprises SEQ ID NO: 138 encoding an abeta antibody or antigen-binding fragment thereof. In some embodiments, a(n isolated) nucleic acid is provided, wherein the (isolated) nucleic acid comprises SEQ ID NO: 139 encoding an abeta antibody or antigen-binding fragment thereof. In some embodiments, a(n isolated) nucleic acid is provided, wherein the (isolated) nucleic acid comprises SEQ ID NO: 148 encoding an abeta antibody or antigen-binding fragment thereof. In some embodiments, a(n isolated) nucleic acid is provided, wherein the (isolated) nucleic acid comprises SEQ ID NO: 149 encoding an abeta antibody or antigen-binding fragment thereof. In some embodiments, a(n isolated) nucleic acid is provided, wherein the (isolated) nucleic acid comprises SEQ ID NO: 158 encoding an abeta antibody or antigen-binding fragment thereof. In some embodiments, a(n isolated) nucleic acid is provided, wherein the (isolated) nucleic acid comprises SEQ ID NO: 159 encoding an abeta antibody or antigen-binding fragment thereof. In some embodiments, a(n isolated) nucleic acid is provided, wherein the (isolated) nucleic acid comprises SEQ ID NO: 168 encoding an abeta antibody or antigen-binding fragment thereof. In some embodiments, a(n isolated) nucleic acid is provided, wherein the (isolated) nucleic acid comprises SEQ ID NO: 169 encoding an abeta antibody or antigen-binding fragment thereof. In some embodiments, a(n isolated) nucleic acid is provided, wherein the (isolated) nucleic acid comprises SEQ ID NO: 178 encoding an abeta antibody or antigen-binding fragment thereof. In some embodiments, a(n isolated) nucleic acid is provided, wherein the (isolated) nucleic acid comprises SEQ ID NO: 179 encoding an abeta antibody or antigen-binding fragment thereof. In some embodiments, a(n isolated) nucleic acid is provided, wherein the (isolated) nucleic acid comprises SEQ ID NO: 188 encoding an abeta antibody or antigen-binding fragment thereof. In some embodiments, a(n isolated) nucleic acid is provided, wherein the (isolated) nucleic acid comprises SEQ ID NO: 189 encoding an abeta antibody or antigen-binding fragment thereof. In some embodiments, a(n isolated) nucleic acid is provided, wherein the (isolated) nucleic acid comprises SEQ ID NO: 199 encoding an abeta antibody or antigen-binding fragment thereof.

In some embodiments, a diagnostic composition is provided, comprising a binding molecule, in particular an antibody or antigen-binding fragment thereof, of the invention (and as provided herein) and an acceptable carrier and/or excipient. For example, the binding molecule, in particular the antibody or antigen-binding fragment thereof, may be combined, as appropriate, with acceptable carriers or media such as sterilized water or saline solution, vegetable oils, emulsifiers, suspensions, surfactants, stabilizers, flavoring agents, excipients, vehicles, preservatives, and binders, for example, and formulated into a diagnostic preparation. Preferred diagnostic compositions of the invention are compatible with use of body fluids as the test sample. Thus, the diagnostic compositions may permit direct use with body fluid samples without requiring further processing of the body fluid sample. For cellular and/or tissue applications, the diagnostic compositions may be identically, or differently, formulated in some embodiments to facilitate use with such samples.

In some embodiments, a diagnostic composition is provided, comprising at least two amyloid-beta binding antibodies or antigen-binding fragments thereof of the invention and an acceptable carrier and/or excipient.

In some embodiments, a diagnostic composition is provided comprising:
a. a first amyloid-beta binding antibody or antigen-binding fragment thereof that selectively binds to any amyloid-beta peptides or species in solution independently of the conformational state of the amyloid-beta peptides or species, and
b. a second amyloid-beta binding antibody or antigen-binding fragment thereof that selectively binds to any amyloid-beta peptides or species in solution independently of the conformational state of the amyloid-beta peptides or species
wherein at least one, or both, of the first and second antibodies displays no cross-reactivity to soluble amyloid precursor protein (APP), in particular with no cross-reactivity to soluble APP alpha.

One or both antibodies may be an antibody of the invention. Either antibody may be used as capture antibody. It may, therefore, be provided for attachment, or attached, to a solid support. The other antibody may be used as detection antibody. It may, therefore, be conjugated with a suitable label. Conjugated antibodies are described herein, which description applies mutatis mutandis.

In some embodiments, both, of the first and second antibodies display no cross-reactivity to soluble amyloid precursor protein (APP), in particular with no cross-reactivity to soluble APP alpha. In other embodiments, one of the first and second antibody or antigen-binding fragment thereof displays cross-reactivity to soluble amyloid precursor protein (APP). The cross-reactivity may be low or high. The other antibody or antigen-binding fragment thereof displays no cross-reactivity to soluble amyloid precursor protein (APP). In some embodiments, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected from:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and/or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; and/or
c) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

In specific embodiments, a diagnostic composition is provided comprising a) and b) above or a) and c) above.

In a more preferred embodiment, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected from:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37.

In some embodiments, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected form:
a) a Heavy Chain Variable Region (VH) comprising the sequence of SEQ ID NO: 10 and a Light Chain Variable Region (VL) comprising the sequence of SEQ ID NO: 14; and/or
b) a Heavy Chain Variable Region (VH) comprising the sequence of SEQ ID NO: 30 and a Light Chain Variable Region (VL) comprising the sequence of SEQ ID NO: 34; and/or
c) a Heavy Chain Variable Region (VH) comprising the sequence of SEQ ID NO: 150 and a Light Chain Variable Region (VL) comprising the sequence of SEQ ID NO: 154.

In specific embodiments, a diagnostic composition is provided comprising a) and b) above or a) and c) above.

In a more preferred embodiments, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected form:
a) a Heavy Chain Variable Region (VH) comprising the sequence of SEQ ID NO: 10 and a Light Chain Variable Region (VL) comprising the sequence of SEQ ID NO: 14; and
b) a Heavy Chain Variable Region (VH) comprising the sequence of SEQ ID NO: 30 and a Light Chain Variable Region (VL) comprising the sequence of SEQ ID NO: 34.

In some embodiments, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected form:
a) a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14; and/or
b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; and/or
c) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154.

In specific embodiments, a diagnostic composition is provided comprising a) and b) above or a) and c) above.

In some embodiments, a diagnostic composition is provided, comprising at least two amyloid-beta binding antibodies or antigen-binding fragments thereof and an acceptable carrier and/or excipient, wherein the amyloid-beta binding antibodies or antigen-binding fragments are selected form:
a) a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14; and
b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34

In some embodiments, a diagnostic composition is provided, comprising at least one amyloid-beta binding antibody or antigen-binding fragment thereof of the invention, in particular an antibody or antigen-binding fragment thereof that displays no cross-reactivity to soluble amyloid precursor protein (APP), in combination with at least one amyloid-beta antibody known in the art and an acceptable carrier and/or excipient. In some embodiments, the amyloid-beta antibody known in the art is a commercially available abeta binding antibody. In some embodiments, the antibody is selected form: NAB228 (Invitrogen), 6E10 (Covance), 2C8 (Invitrogen), 4G8 (Chemicon), WO-2 (Merck) and DE2 (Chemicon). Such antibodies may be used as capture antibody and the antibody of the invention used as detection antibody (or vice versa).

In some embodiments, a conjugated binding molecule, in particular antibody or antigen-binding fragment thereof, is provided, comprising a binding molecule, in particular an antibody or antigen-binding fragment thereof, described herein and a conjugated molecule. Conjugates of the invention may be referred to as immunoconjugates. Any suitable conjugated molecule may be employed according to the invention. Suitable examples include, but are not limited to enzymes (e.g. alkaline phosphatase or horseradish peroxidase), avidin, streptavidin, biotin, Protein A/G, magnetic beads, fluorophores, radioactive isotopes (i.e., radioconjugates), nucleic acid molecules, detectable labels, therapeutic agents, toxins and blood brain barrier penetration moieties. Conjugation methods are well known in the art and several technologies are commercially available for conjugating antibodies to a label or other molecule. Conjugation is typically through amino acid residues contained within the binding molecules of the invention (such as lysine, histidine or cysteine). They may rely upon methods such as the NHS (Succinimidyl) ester method, isothiocyanate method, carbodiimide method and periodate method. Conjugation may be achieved through creation of fusion proteins for example. This is appropriate where the binding molecule is conjugated with another protein molecule. Thus, suitable genetic constructs may be formed that permit the expression of a fusion of the binding molecule of the invention with the label or other molecule. Conjugation may be via a suitable linker moiety to ensure suitable spatial separation of the antibody and conjugated molecule, such as detectable label. However, a linker may not be required in all instances.

In some embodiments, a diagnostic composition is provided, comprising an isolated binding molecule, in particular an antibody or antigen-binding fragment thereof, described herein and an acceptable carrier and/or excipient.

The binding molecules, in particular amyloid-beta binding antibody or antigen-binding fragment thereof, of the invention have many uses. They may be employed for research use, in particular as an analytical tool or reference molecule. They may be used in detecting amyloid-beta aggregates, including plaques, in vitro or in vivo. The binding molecules may be used to stain amyloid-beta aggregates. For example, the binding molecules may be used for histochemical detection in postmortem brain tissue. The binding molecules are typically labelled and may be directly or indirectly labelled as discussed herein. Such uses may be performed in relation to any suitable animal, in particular mammal. Preferred subjects are human and mice.

In some embodiments, a method of diagnosing an amyloid-beta associated disease, disorder or condition, such as Alzheimer's Disease (AD), mild cognitive impairment (MCI), Down syndrome (DS), Down syndrome-related Alzheimer's Disease, cardiac amyloidosis, cerebral amyloid angiopathy (CAA), multiple sclerosis, Parkinson's disease (PD), Parkinson's Disease with Dementia (PDD), Lewy body dementia, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure is provided. The method comprises administering a binding molecule, in particular an antibody or an antigen-binding fragment thereof, of the invention or a diagnostic composition of the invention to the subject. More particularly in some embodiments, a method of diagnosing an amyloid-beta associated disease, disorder or condition is provided, wherein the amyloid-beta associated disease, disorder or condition is selected from the group consisting of Alzheimer's Disease (AD), Down syndrome (DS), Down syndrome-related Alzheimer's Disease, cerebral amyloid angiopathy (CAA), Myotonic dystrophy and Lewy body dementia. The subject is typically a mammalian subject, preferably a human. For in vivo methods, the binding molecule is typically labelled with a suitable label for visualization purposes.

In certain embodiments, a binding molecule, in particular an antibody or antigen-binding fragment thereof, of the invention and as provided herein is useful for detecting the presence of abeta in a biological sample. In certain embodiments, a binding molecule, in particular an antibody or antigen-binding fragment thereof, of the invention and as provided herein is useful for detecting the presence of abeta in a body fluid sample. The term “body fluid sample” includes samples derived from a body fluid by further processing, such as concentration, centrifugation and addition of further substances such as buffers and preservatives. In particular embodiments, a binding molecule, in particular an antibody or antigen-binding fragment thereof, of the invention as provided herein is useful for detecting the presence of pathological abeta in a biological sample. The term “detecting” as used herein encompasses quantitative and/or qualitative detection. The biological sample is typically obtained from a mammal, in particular a human. The biological sample is typically a clinical sample from a subject suspected of, or being screened for, an amyloid-beta associated disease, disorder or condition according to any of the methods described herein. The obtaining of a sample does not form an essential step of the methods of the invention, which may therefore begin with an already isolated sample. Obtaining a sample may be performed according to any suitable technique, determined by the sample in question. In certain embodiments, a biological sample comprises a cell, tissue and/or a body fluid (nasal secretions, urine, blood, etc.) from the subject. Examples of suitable biological samples include cerebrospinal fluid (CSF), interstitial fluid (ISF), a cell or tissue of the brain (e.g., brain cortex or hippocampus). Examples of suitable body fluids include cerebrospinal fluid (CSF), interstitial fluid (ISF), blood (to include whole blood and derivatives such as serum and plasma), urine and nasal secretions. In some embodiments, a biological sample is cerebrospinal fluid (CSF) or blood. In some embodiments, the biological sample is plasma.

In certain embodiments, a method for detecting abeta in a biological sample is provided, wherein the method comprises contacting the biological sample with a binding molecule, in particular an abeta antibody or antigen-binding fragment thereof, of the invention as provided herein under conditions permissive for binding of the binding molecule, in particular the abeta antibody or antigen-binding fragment thereof, to abeta, and detecting whether a complex is formed between the binding molecule, in particular the abeta antibody or antigen-binding fragment thereof, and abeta. If formation of a complex is detected, it may be determined that the sample contains abeta. In the absence of complex formation or complex formation below a pre-determined threshold, it may be determined that the sample is free of abeta. Such method may be an in vitro or in vivo method. Further, the complex formed between the binding molecule, the abeta antibody or the antigen-binding fragment thereof, and abeta in a test biological sample can be compared to the complex formed in a control biological sample (e.g., a biological sample from a healthy subject or subjects). The amount of the complex formed between the binding molecule, the abeta antibody or the antigen-binding fragment thereof, and abeta in a test biological sample can also be quantified and compared to the amount of the complex formed in a control biological sample (e.g., a biological sample from a healthy subject or subjects) or to the average amount of the complex known to be formed in healthy subjects.

In certain embodiments, a binding molecule, in particular an antibody or antigen-binding fragment thereof, of the invention and as provided herein is useful for detecting the presence of abeta in a biological sample. In particular embodiments, the binding molecule, in particular an antibody or antigen-binding fragment thereof, of the invention and as provided herein is useful as an assay reagent, positive control, biomarker detection reagent and/or calibrator for an immunoassay, (including, but not limited to an ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) or any other assay technology or kit that relies on antibodies for target immunocapture and/or detection). As such, the binding molecule, in particular antibody or antigen-binding fragments thereof, may be used in assays for validating/screening binding molecules, abeta or abeta fragments, abeta antibodies or antigen-binding fragments thereof. The binding molecules, in particular an antibody or antigen-binding fragment thereof, of the invention may be used as detection tools and/or positive controls as they bind to all abeta species in the sample in selective fashion. In one embodiment, at least two amyloid-beta antibodies or antigen-binding fragments thereof may be used as assay reagents, positive controls, biomarker detection reagent and/or calibrators for an immunoassay as listed above. In one embodiment, at least two amyloid-beta antibodies or antigen-binding fragments thereof may be used in an immunoassay, preferably a sandwich assay (ELISA), resulting in a sandwich pairing of antibodies, wherein preferably the antibodies are selected from ACI-24-41F12-Ab3, ACI-31-25B1-Ab2 and ACI-8041-5A2D4-Ab1, even more preferably the antibodies are ACI-24-41F12-Ab3 and ACI-31-25B1-Ab2. In one embodiment, the at least two amyloid-beta antibodies or antigen-binding fragments thereof may be used in a sandwich assay (ELISA), resulting in a sandwich pairing of antibodies, wherein one antibody or antigen-binding fragment thereof is used for capturing the target (including but not limited to abeta), preferably ACI-31-25B1-Ab2, and wherein another antibody or antigen-binding fragment thereof is used for detecting the captured target, preferably ACI-24-41F12-Ab3, more preferably ACI-24-41F12-Ab3 that is conjugated to a detectable label.

The invention relates to a method of detecting amyloid-beta in a sample obtained from a subject, the method comprising:
a. capturing amyloid beta with an amyloid-beta binding antibody or antigen-binding fragment thereof that selectively binds to any amyloid-beta peptides or species in solution independently of the conformational state of the amyloid-beta peptides or species, and
b. detecting captured amyloid beta with an amyloid-beta binding antibody or antigen-binding fragment thereof that selectively binds to any amyloid-beta peptides or species in solution independently of the conformational state of the amyloid-beta peptides or species
   wherein at least one, or both, of the capture (a) and detection (b) antibody or antigen-binding fragment thereof displays no cross-reactivity to soluble amyloid precursor protein (APP), in particular with no cross-reactivity to soluble APP alpha. One or both of the capture and detection antibodies may be an antibody of the invention. The capture antibody may be provided for attachment, or attached, to a solid support. The detection antibody may be conjugated with a suitable label. Conjugated antibodies are described herein, which description applies mutatis mutandis.

In some embodiments, both the capture and detection antibodies display no cross-reactivity to soluble amyloid precursor protein (APP), in particular with no cross-reactivity to soluble APP alpha. In other embodiments, one of the capture and detection antibody or antigen-binding fragment thereof displays cross-reactivity to soluble amyloid precursor protein (APP). The cross-reactivity may be low or high. The other antibody or antigen-binding fragment thereof displays no cross-reactivity to soluble amyloid precursor protein (APP).

In some embodiments, the capture antibody or antigen-binding fragment is immobilized on a solid support, such as on the surface of a well (e.g. in a multiwell plate). In some embodiments, the detection antibody or antigen-binding fragment is labelled with a detectable label.

In some embodiments, the capture or detection (preferably detection) amyloid-beta binding antibody or antigen-binding fragment comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17. In some embodiments, the capture or detection (preferably detection) amyloid-beta binding antibody or antigen-binding fragment comprises a Heavy Chain Variable Region (VH) comprising the sequence of SEQ ID NO: 10 and a Light Chain Variable Region (VL) comprising the sequence of SEQ ID NO: 14. In some embodiments, the capture or detection (preferably detection) amyloid-beta binding antibody or antigen-binding fragment comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14

In some embodiments, the capture or detection (preferably capture) amyloid-beta binding antibody or antigen-binding fragment comprises:
a. a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
b. a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

The preferred capture amyloid-beta binding antibody or antigen-binding fragment comprises a) above.

In some embodiments, the capture or detection (preferably capture) amyloid-beta binding antibody or antigen-binding fragment comprises:
a) a Heavy Chain Variable Region (VH) comprising the sequence of SEQ ID NO: 30 and a Light Chain Variable Region (VL) comprising the sequence of SEQ ID NO: 34; or
b) a Heavy Chain Variable Region (VH) comprising the sequence of SEQ ID NO: 150 and a Light Chain Variable Region (VL) comprising the sequence of SEQ ID NO: 154.

In some embodiments, the capture or detection (preferably capture) amyloid-beta binding antibody or antigen-binding fragment comprises:
a) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; and/or
b) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154.

The invention also relates to a method of detecting amyloid-beta in a sample obtained from a subject, the method comprising:
a. capturing amyloid beta with an amyloid-beta binding antibody or antigen-binding fragment thereof that selectively binds to any amyloid-beta peptides or species in solution independently of the conformational state of the amyloid-beta peptides or species and displays no cross-reactivity to soluble amyloid precursor protein (APP), in particular with high cross-reactivity to soluble APP alpha; and
b. detecting captured amyloid beta with an amyloid-beta binding antibody or antigen-binding fragment thereof that selectively binds to any amyloid-beta peptides or species in solution independently of the conformational state of the amyloid-beta peptides or species and displays cross-reactivity (low or high) to soluble amyloid precursor protein (APP), in particular with no cross-reactivity to soluble APP alpha.

In some embodiments, the capture antibody or antigen-binding fragment is immobilized on a solid support, such as on the surface of a well (e.g. in a multiwell plate). In some embodiments, the capture and/or detection antibody or antigen-binding fragment has high affinity for abeta. In some embodiments, the detection antibody or antigen-binding fragment is labelled with a detectable label. In some embodiments, the capture amyloid-beta binding antibody or antigen-binding fragment comprises:
a. a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
b. a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

The preferred capture amyloid-beta binding antibody or antigen-binding fragment comprises a) above.

In some embodiments, the capture amyloid-beta binding antibody or antigen-binding fragment comprises:
a) a Heavy Chain Variable Region (VH) comprising the sequence of SEQ ID NO: 30 and a Light Chain Variable Region (VL) comprising the sequence of SEQ ID NO: 34; or
b) a Heavy Chain Variable Region (VH) comprising the sequence of SEQ ID NO: 150 and a Light Chain Variable Region (VL) comprising the sequence of SEQ ID NO: 154.

In some embodiments, the capture amyloid-beta binding antibody or antigen-binding fragment comprises:
a) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; and/or
   a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154. In some embodiments, the detection amyloid-beta binding antibody or antigen-binding fragment comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17. In some embodiments, the detection amyloid-beta binding antibody or antigen-binding fragment comprises a Heavy Chain Variable Region (VH) comprising the sequence of SEQ ID NO: 10 and a Light Chain Variable Region (VL) comprising the sequence of SEQ ID NO: 14. In some embodiments, the detection amyloid-beta binding antibody or antigen-binding fragment comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14.

In some embodiments, the invention comprises a method of detecting amyloid-beta in a sample obtained from a subject, the method comprising the step of capturing amyloid beta with a first amyloid-beta binding antibody or fragment thereof and the step of detecting captured amyloid-beta in the sample with a second amyloid-beta binding antibody or fragment thereof, wherein each amyloid-beta binding antibody or antigen-binding fragment thereof is independently selected from: ACI-24-41F12-Ab3, ACI-31-25B1-Ab2 and ACI-8041-5A2D4-Ab1, preferably the antibodies are ACI-24-41F12-Ab3 and ACI-31-25B1-Ab2, more preferably ACI-24-41F12-Ab3 that is conjugated to a detectable label. In another embodiment of the invention, the step of capturing amyloid beta is performed with a first amyloid-beta binding antibody or fragment thereof selected from ACI-24-41F12-Ab3, ACI-31-25B1-Ab2 and ACI-8041-5A2D4-Ab1, preferably the antibodies are ACI-24-41F12-Ab3 and ACI-31-25B1-Ab2, more preferably ACI-31-25B1-Ab2. In another embodiment of the invention, the step of detecting the captured amyloid beta is performed with a second amyloid-beta binding antibody or fragment thereof that is conjugated to a detectable label and selected from ACI-24-41F12-Ab3, ACI-31-25B1-Ab2 and ACI-8041-5A2D4-Ab1, preferably the antibodies are ACI-24-41F12-Ab3 and ACI-31-25B1-Ab2, more preferably ACI-24-41F12-Ab3. In some embodiments, the step of detecting captured amyloid-beta in the sample is performed with a second amyloid-beta binding antibody or fragment thereof, wherein each amyloid-beta binding antibody or antigen-binding fragment thereof is conjugated to a detectable label known in the art.

The invention therefore provides a method of detecting amyloid-beta in a sample obtained from a subject, the method comprising contacting the sample with a binding molecule, in particular an antibody or antigen-binding fragment of the invention and detecting binding of the antibody or antigen-binding fragment thereof in order to detect amyloid-beta in the sample. Similarly, the invention provides a method of quantifying amyloid-beta in a sample obtained from a subject, the method comprising contacting the sample with a binding molecule, in particular an antibody or antigen-binding fragment of the invention and performing quantification based on the binding of the binding molecule to amyloid-beta. This method may comprise comparing the amyloid-beta levels in the sample to those in a control sample or samples. The levels in control samples represent known levels against which the levels in the test sample may be determined. The control samples are not, therefore, necessarily tested at the same time as the method of quantification is performed. However, in some embodiments, reference levels are determined in parallel with the test sample. For example, a quantitative ELISA, ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020) may be performed. A standard curve may be generated to permit quantification based on a dilution series (serial dilution) of abeta. Diagnostic compositions of the invention may be used in such methods. The sandwich immunoassays described herein, incorporating a suitable capture and detection antibody or antigen binding fragment thereof, may be used in the methods of quantifying amyloid-beta in a sample obtained from a subject.

The invention also provides a method for diagnosing a disease, disorder and/or condition associated with amyloid-beta comprising contacting the sample with a binding molecule, in particular an antibody or antigen-binding fragment of the invention and comparing the amyloid-beta levels in the sample to those in a control sample or samples. Higher levels of amyloid-beta in the sample compared with a control level based on healthy subjects are indicative of a disease, disorder and/or condition associated with amyloid-beta. Additionally or alternatively similar or higher levels of amyloid-beta in the sample compared with a diseased control (i.e. one or more samples from a subject having the disease, disorder and/or condition associated with amyloid-beta) are indicative of a disease, disorder and/or condition associated with amyloid-beta. Diagnostic compositions of the invention may be used in such methods. The sandwich immunoassays described herein, incorporating a suitable capture and detection antibody or antigen binding fragment thereof, may be used in the methods of diagnosing a disease, disorder and/or condition associated with amyloid-beta.

The binding molecules of the invention are also useful in classification methods, for example, to indicate the relative stage of the disease, disorder and/or condition associated with amyloid-beta. The invention therefore also provides a method for classifying a disease, disorder and/or condition associated with amyloid-beta comprising contacting a sample from a subject with a binding molecule, in particular an antibody or antigen-binding fragment of the invention and comparing the amyloid-beta levels in the sample to those in a control sample or samples in order to classify the disease. A range of controls representative of different classes of disease may be employed in order to classify the sample. The test sample may be classified based on the best match to the control samples. Higher levels of amyloid-beta in the sample compared with a control level based on healthy subjects are indicative of a disease, disorder and/or condition associated with amyloid-beta. Similar or higher levels of amyloid-beta in the sample compared with a diseased control at a certain stage of disease are indicative of that stage of disease, disorder and/or condition associated with amyloid-beta. Such methods may be performed in relation to subjects known to have the disease, disorder and/or condition associated with amyloid-beta and/or in relation to subjects not already known to have the disease, disorder and/or condition associated with amyloid-beta. Diagnostic compositions of the invention may be used in such methods. The sandwich immunoassays described herein, incorporating a suitable capture and detection antibody or antigen binding fragment thereof, may be used in the classification methods of the invention.

The invention also provides a method for monitoring a disease, disorder and/or condition associated with amyloid-beta at two or more time points using samples from a subject, the method comprising contacting the samples with a binding molecule, in particular an antibody or antigen-binding fragment of the invention and comparing the amyloid-beta levels in the samples, wherein higher levels of amyloid-beta in the later sample compared with one or more earlier samples are indicative of progression of a disease, disorder and/or condition associated with amyloid-beta. Similarly, the invention provides a method for monitoring a disease, disorder and/or condition associated with amyloid-beta at two or more time points using samples from a subject, the method comprising contacting the samples with a binding molecule, in particular an antibody or antigen-binding fragment of the invention and comparing the amyloid-beta levels in the samples, wherein lower levels of amyloid-beta in the later sample compared with one or more earlier samples are indicative of regression of a disease, disorder and/or condition associated with amyloid-beta. These methods also permit a lack of progression of the disease to be monitored, where there is no significant change in levels of amyloid-beta in the later sample compared with one or more earlier samples. Such methods are typically performed in relation to subjects known to have the disease, disorder and/or condition associated with amyloid-beta. Diagnostic compositions of the invention may be used in such methods. The sandwich immunoassays described herein, incorporating a suitable capture and detection antibody or antigen binding fragment thereof, may be used in the monitoring methods of the invention.

Monitoring methods are useful to determine whether a particular therapy is successful or otherwise. The invention therefore also provides a method for monitoring a disease, disorder and/or condition associated with amyloid-beta at two or more time points using samples from a subject, the method comprising contacting the samples with a binding molecule, in particular an antibody or antigen-binding fragment of the invention, wherein lower levels of amyloid-beta in the later sample compared with one or more earlier samples are indicative of successful treatment of a disease, disorder and/or condition associated with amyloid-beta. The therapy may be any suitable candidate therapeutic agent, such as an antibody or small molecule therapeutic. These methods also permit a lack of progression of the disease to be monitored, where there is no significant change in levels of amyloid-beta in the later sample compared with one or more earlier samples. This may also be considered successful treatment in some circumstances. Indeed, a decline in the rate of increase of abeta levels between samples, compared with the rate of increase prior to therapy, may also be considered indicative of successful treatment. Such methods are typically performed in relation to subjects known to have the disease, disorder and/or condition associated with amyloid-beta. Unsuccessful treatment may be determined where the treatment provides no decline in the rate of increase of abeta levels between samples, compared with the rate of increase prior to therapy. Diagnostic compositions of the invention may be used in such methods. The sandwich immunoassays described herein, incorporating a suitable capture and detection antibody or antigen binding fragment thereof, may be used in the methods of monitoring therapy of the invention.

The binding molecules of the invention may also be used to assist with therapy selection. Thus, the invention provides a method for selecting a therapy for treatment of a disease, disorder and/or condition associated with amyloid-beta, the method comprising contacting samples taken before and after treatment with a binding molecule, in particular an antibody or antigen-binding fragment of the invention, wherein lower levels of amyloid-beta in the sample taken after treatment compared with the sample taken before treatment are indicative of successful treatment of a disease, disorder and/or condition associated with amyloid-beta and thus the therapy is selected for treatment. The therapy may be any suitable candidate therapeutic agent, such as an antibody or small molecule therapeutic. A therapy halting progression of the disease may also be selected, where there is no significant change in levels of amyloid-beta in the later sample compared with one or more earlier samples. This may also be considered successful treatment in some circumstances. Indeed, a decline in the rate of increase of abeta levels between samples, compared with the rate of increase prior to therapy, may also be considered indicative of successful treatment and therefore result in selection of the particular therapy. Such methods are typically performed in relation to subjects known to have the disease, disorder and/or condition associated with amyloid-beta. Unsuccessful treatment may be determined where the treatment provides no decline in the rate of increase of abeta levels between samples, compared with the rate of increase prior to therapy. Such therapy is not selected for treatment. Alternatively, higher levels of amyloid-beta in the sample taken after treatment compared with the sample taken before may be indicative of unsuccessful treatment of a disease, disorder and/or condition associated with amyloid-beta and thus the therapy is not selected for treatment. Diagnostic compositions of the invention may be used in such methods. The sandwich immunoassays described herein, incorporating a suitable capture and detection antibody or antigen binding fragment thereof, may be used in the therapy selection methods of the invention (as applied to individual subjects).

Methods of the invention are also useful to determine whether a particular therapy is successful or otherwise in the context of a larger, controlled study, such as a clinical trial. Thus, these methods are typically applied to a treatment group of subjects that is compared with a group of subjects not treated with the therapy. In such a context, control samples not treated with the therapy are also available for comparative purposed (placebo group). The invention therefore also provides a method for assessing a candidate therapy for a disease, disorder and/or condition associated with amyloid-beta, the method comprising, following treatment of one or more subjects, contacting samples from the one or more treated subjects with a binding molecule, in particular an antibody or antigen-binding fragment of the invention, wherein lower levels of amyloid-beta in the samples compared with levels in corresponding samples from subjects not treated with the therapy are indicative of successful treatment of a disease, disorder and/or condition associated with amyloid-beta. The methods are typically performed in relation to a plurality (i.e. at least two) treated subjects and a plurality of control subjects. The treated and control groups may or may not be of the same size. They may each comprise 3 or more, 4 or more, 5 or more, 10 or more, 20 or more, 50 or more subjects in some embodiments. The therapy may be any suitable candidate therapeutic agent, such as a biologic, in particular an antibody, a vaccine or small molecule therapeutic. The methods may be performed at multiple time points in matched samples between the treatment and placebo groups in order to monitor the effectiveness of the candidate therapy over a defined time period. An initial pre-therapy sample is typically also taken. Thus, the methods may comprise contacting samples from the one or more treated subjects and the subjects not treated with the therapy with a binding molecule, in particular an antibody or antigen-binding fragment of the invention prior to treatment to determine base levels of amyloid-beta. “Prior to treatment” means prior to administration of the therapy or the placebo depending upon the subject group. The binding molecules of the invention may therefore also be used to assist with assessment of candidate therapies in the context of clinical trials. Candidate therapies providing successful treatment may be selected and, ultimately, approved for marketing. Diagnostic compositions of the invention may be used in such methods. The sandwich immunoassays described herein, incorporating a suitable capture and detection antibody or antigen binding fragment thereof, may be used in the therapy selection methods of the invention (as applied to clinical trials).

According to all of the relevant methods, the disease, disorder and/or condition associated with amyloid-beta may be selected from the group consisting of Alzheimer's Disease (AD), mild cognitive impairment (MCI), Down syndrome (DS), Down syndrome-related Alzheimer's Disease, cardiac amyloidosis, cerebral amyloid angiopathy (CAA), multiple sclerosis, Parkinson's disease, Parkinson's Disease with Dementia (PDD), Lewy body dementia, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure. In specific embodiments, the amyloid-beta associated disease, disorder or condition is Alzheimer's Disease (AD), Down syndrome (DS), Down syndrome-related Alzheimer's Disease, cerebral amyloid angiopathy (CAA), Myotonic dystrophy, or Lewy body dementia.

Suitable samples employed with the various methods are typically biological samples, particularly body fluids as discussed herein. Suitable immunoassay formats are known to the skilled person including, but not limited to an ELISA, MSD (Meso Scale Discovery Inc., USA), Luminex (Luminex Corp., USA), Alphalisa (PerkinElmer, Inc., USA), Gyrolab (Gyros Protein Technologies AB, Sweden), Simoa (Quanterix Corp., USA), Gyros™ (Given et al., 2012), Singulex Erenna (EMD Millipore, Corp., USA), iR-SENSE/Immuno-InfraRed assay (Nabers et al, 2016), MITOMI (Piraino et al, 2016), Immunoprecipitation combined with liquid chromatography mass spectrometry (IP LC-MS/MS; Shimadzu, Germany), Surface plasmon resonance (SPR; Cytiva Europe, Switzerland), Atomic force microscope (AFM) (Kiio and Park, 2020).

In some embodiments, a binding molecule, an abeta antibody or antigen-binding fragment thereof, of the invention is part of a diagnostic kit comprising such a binding molecule, abeta antibody or antigen-binding fragment thereof. Such kits may comprise all necessary components for performing the herein provided methods and/or assays, such as, for example, buffers, detectable dyes, laboratory equipment, reaction containers, instructions and the like. The binding molecules, in particular antibodies or antigen-binding fragment thereof, may be conjugated with a label or other molecule as discussed herein. They may be labelled or expressed with dyes or tags, such as fluorophores or fluorescent dyes, or be coupled to an enzyme or to a solid support (such as an assay chip), as required for each corresponding assay technology. In a further embodiment, a binding molecule, in particular an antibody or antigen-binding fragment thereof, of the invention may be conjugated to radioactive isotopes (i.e., radioconjugates), nucleic acid molecules, detectable labels, therapeutic agents, toxins or to blood brain barrier penetration moieties, forming immunoconjugates.

The kits may be used to perform any of the methods of the invention and thus may incorporate any suitable components, including reagents, necessary to perform those methods. The invention is further directed to a kit comprising an abeta antibody or antigen-binding fragment thereof of the invention. The kits typically contain the abeta antibody or antigen-binding fragment thereof of the invention in a suitable container. The abeta antibody or antigen-binding fragment thereof may be provided in the form of a diagnostic composition ready for application or it may be provided in the kit with suitable agents for reconstitution of the product, such as saline solution. The kit may be provided with instructions for use. The discussion of the methods of the invention therefore applies mutatis mutandis. Kits for performing the sandwich immunoassays of the invention are also provided. They incorporate a suitable capture and detection antibody or antigen binding fragment thereof. Thus, the invention relates to a kit for detecting amyloid-beta in a sample obtained from a subject, the kit comprising:
a. a capture amyloid-beta binding antibody or antigen-binding fragment thereof that selectively binds to any amyloid-beta peptides or species in solution independently of the conformational state of the amyloid-beta peptides or species; and
b. a detection amyloid-beta binding antibody or antigen-binding fragment thereof that selectively binds to any amyloid-beta peptides or species in solution independently of the conformational state of the amyloid-beta peptides or species
wherein at least one of the capture and detection antibody or antigen-binding fragment thereof displays no cross-reactivity to soluble amyloid precursor protein (APP), in particular with no cross-reactivity to soluble APP alpha.

One or both antibodies may be an antibody of the invention.

In some embodiments, the capture antibody or antigen-binding fragment is immobilized on a solid support, such as on the surface of a well (e.g. in a multiwell plate) or is provided with a solid support and/or means for immobilizing the capture antibody or antigen-binding fragment on a solid support. In some embodiments, the detection antibody or antigen-binding fragment is labelled with a detectable label or is provided with a label and means for conjugating the antibody to the label, either directly or indirectly.

In some embodiments, both of the capture and detection antibodies display no cross-reactivity to soluble amyloid precursor protein (APP), in particular with no cross-reactivity to soluble APP alpha. In other embodiments, one of the capture and detection antibody or antigen-binding fragment thereof displays cross-reactivity to soluble amyloid precursor protein (APP). The cross-reactivity may be low or high. The other antibody or antigen-binding fragment thereof (either capture or detection antibody) displays no cross-reactivity to soluble amyloid precursor protein (APP).

In some embodiments, the capture or detection (preferably detection) amyloid-beta binding antibody or antigen-binding fragment comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17. In some embodiments, the capture or detection (preferably detection) amyloid-beta binding antibody or antigen-binding fragment comprises a Heavy Chain Variable Region (VH) comprising the sequence of SEQ ID NO: 10 and a Light Chain Variable Region (VL) comprising the sequence of SEQ ID NO: 14. In some embodiments, the capture or detection (preferably detection) amyloid-beta binding antibody or antigen-binding fragment comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14.

In some embodiments, the capture or detection (preferably capture) amyloid-beta binding antibody or antigen-binding fragment comprises:
a. a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
b. a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

The preferred capture amyloid-beta binding antibody or antigen-binding fragment comprises a) above.

In some embodiments, the capture or detection (preferably capture) amyloid-beta binding antibody or antigen-binding fragment comprises:
a) a Heavy Chain Variable Region (VH) comprising the sequence of SEQ ID NO: 30 and a Light Chain Variable Region (VL) comprising the sequence of SEQ ID NO: 34; or
b) a Heavy Chain Variable Region (VH) comprising the sequence of SEQ ID NO: 150 and a Light Chain Variable Region (VL) comprising the sequence of SEQ ID NO: 154.

In some embodiments, the capture or detection (preferably capture) amyloid-beta binding antibody or antigen-binding fragment comprises:
a) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; and/or
b) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154.

The invention also relates to a kit for detecting amyloid-beta in a sample obtained from a subject, the kit comprising:
a. a capture amyloid-beta binding antibody or antigen-binding fragment thereof that selectively binds to any amyloid-beta peptides or species in solution independently of the conformational state of the amyloid-beta peptides or species and displays no cross-reactivity to soluble amyloid precursor protein (APP), in particular with high cross-reactivity to soluble APP alpha; and
b. A detection amyloid-beta binding antibody or antigen-binding fragment thereof that selectively binds to any amyloid-beta peptides or species in solution independently of the conformational state of the amyloid-beta peptides or species and displays cross-reactivity (low or high) to soluble amyloid precursor protein (APP), in particular with no cross-reactivity to soluble APP alpha.

In some embodiments, the capture antibody or antigen-binding fragment is immobilized on a solid support, such as on the surface of a well (e.g. in a multiwell plate) or is provided with a solid support and/or means for immobilizing the capture antibody or antigen-binding fragment on a solid support. In some embodiments, the detection antibody or antigen-binding fragment has high affinity for abeta. In some embodiments, the detection antibody or antigen-binding fragment is labelled with a detectable label or is provided with a label and means for conjugating the antibody to the label, either directly or indirectly. In some embodiments, the capture amyloid-beta binding antibody or antigen-binding fragment comprises:
a. a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
b. a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

The preferred capture amyloid-beta binding antibody or antigen-binding fragment comprises a) above.

In some embodiments, the capture amyloid-beta binding antibody or antigen-binding fragment comprises:
a) a Heavy Chain Variable Region (VH) comprising the sequence of SEQ ID NO: 30 and a Light Chain Variable Region (VL) comprising the sequence of SEQ ID NO: 34; or
b) a Heavy Chain Variable Region (VH) comprising the sequence of SEQ ID NO: 150 and a Light Chain Variable Region (VL) comprising the sequence of SEQ ID NO: 154.

In some embodiments, the capture amyloid-beta binding antibody or antigen-binding fragment comprises:
a) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; and/or
b) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154.

In some embodiments, the detection amyloid-beta binding antibody or antigen-binding fragment comprises a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17. In some embodiments, the detection amyloid-beta binding antibody or antigen-binding fragment comprises a Heavy Chain Variable Region (VH) comprising the sequence of SEQ ID NO: 10 and a Light Chain Variable Region (VL) comprising the sequence of SEQ ID NO: 14. In some embodiments, the detection amyloid-beta binding antibody or antigen-binding fragment comprises a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14.

The invention may be further defined in the following numbered clauses:
1. An antibody or antigen-binding fragment thereof comprising a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17, wherein the antibody or antigen-binding fragment thereof binds to amyloid-beta.
2. An antibody or antigen-binding fragment thereof of clause 1, wherein the antibody or antigen-binding fragment binds to amino acids residues 1-8 (SEQ ID NO: 2) of human amyloid beta SEQ ID NO: 1.
3. The antibody or antigen-binding fragment thereof of clauses 1-2, wherein the antibody comprises a Heavy Chain Variable Region (VH) comprising the sequence of SEQ ID NO: 10 or a heavy chain variable region (VH) having at least 96%, 97%, 98%, or 99%, sequence identity to the amino acid sequence of SEQ ID NO: 10.
4. The antibody or antigen-binding fragment thereof of any of the preceding clauses, wherein the antibody comprises a Heavy Chain Variable Region (VH) comprising the sequence of SEQ ID NO: 10 or a heavy chain variable region (VH) having at least 96%, 97%, 98%, or 99% sequence identity to the amino acid sequence of SEQ ID NO: 10; and a Light Chain Variable Region (VL) comprising the sequence of SEQ ID NO: 14.
5. The antibody or antigen-binding fragment thereof of any of the preceding clauses, wherein the antibody comprises a Heavy Chain Variable Region (VH) comprising the sequence of SEQ ID NO: 10 and a Light Chain Variable Region (VL) comprising the sequence of SEQ ID NO: 14.
6. The antibody or antigen-binding fragment thereof of any of the preceding clauses, wherein the antibody comprises a Heavy Chain (HC) comprising the amino sequence of SEQ ID NO: 20, a Light Chain (LC) comprising the amino sequence of SEQ ID NO: 21.
7. The antibody or antigen-binding fragment thereof of any of the preceding clauses, wherein the antibody comprises a Heavy Chain (HC) comprising the amino sequence of SEQ ID NO: 22 and a Light Chain (LC) comprising the amino sequence of SEQ ID NO: 23.
8. The antibody or antigen-binding fragment thereof of any of the preceding clauses, wherein the antibody comprises a Heavy Chain (HC) comprising the amino sequence of SEQ ID NO: 24 a Light Chain (LC) comprising the amino sequence of SEQ ID NO: 25.
9. The antibody or antigen-binding fragment thereof of any of the preceding clauses, for use in the diagnosis of an amyloid-beta associated disease or condition in a subject.
10. The antibody or antigen-binding fragment thereof of any of the preceding clauses, for use in the detection of amyloid-beta in a sample.
11. The antibody or antigen-binding fragment thereof of any of the preceding clauses, for use in the detection of amyloid-beta in a sample, wherein the sample is a saliva, urine, blood, brain and/or CSF sample, more particularly a blood and/or CSF sample.
12. The antibody or antigen-binding fragment thereof of any of the preceding clauses, for use in the diagnosis of an amyloid-beta associated disease or condition wherein the amyloid-beta associated disease or condition is selected from the group consisting of Alzheimer's Disease (AD), mild cognitive impairment (MCI), Down syndrome, Down syndrome-related Alzheimer's Disease, cardiac amyloidosis, cerebral amyloid angiopathy (CAA), multiple sclerosis, Parkinson's disease, Lewy body dementia, ALS (amyotrophic lateral sclerosis), Adult Onset Diabetes, inclusion body myositis (IBM), ocular amyloidosis, glaucoma, macular degeneration, lattice dystrophy and optic neuritis.
13. The antibody or antigen-binding fragment thereof of any of the preceding clauses, for use in the diagnosis of an amyloid-beta associated disease or condition wherein the amyloid-beta associated disease or condition is Alzheimer's Disease (AD), Down syndrome, Down syndrome-related Alzheimer's Disease, cerebral amyloid angiopathy (CAA), Myotonic dystrophy, Lewy body dementia.
14. The antibody or antigen-binding fragment thereof of any of the preceding clauses, for use in the diagnosis of an amyloid-beta associated disease or condition wherein the amyloid-beta associated disease or condition is Alzheimer's Disease (AD).
15. The antibody or antigen-binding fragment thereof of any of the clauses 1 to 13, for use in the diagnosis of an amyloid-beta associated disease or condition wherein the amyloid-beta associated disease or condition is Down Syndrome.
16. A diagnostic composition comprising an isolated antibody or antigen-binding fragment thereof of any of the preceding clauses and an acceptable carrier and/or excipient.
17. An isolated nucleic acid encoding an antibody of any of the clauses 1-15.
18. A nucleic acid comprising a nucleotide sequence as provided in SEQ ID NO: 18 or SEQ ID NO: 19.
19. A recombinant vector comprising the nucleic acid of clauses 17 or 18.
20. A host cell comprising the nucleic acid of clauses 17 or 18 and/or the vector of clause 19.
21. An isolated host cell that expresses the antibody or antigen-binding fragment of any of the clauses 1-15.
22. A method for producing an isolated binding molecule, in particular an antibody comprising the steps of: a) culturing the host cell of clauses 20 or 21 under conditions suitable for producing the antibody or antigen-binding fragment thereof, and b) isolating the antibody or antigen-binding fragment thereof.

The invention may be further defined in the following numbered clauses:
1. An amyloid-beta binding antibody or antigen-binding fragment thereof comprising:
   a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
   b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
   c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47.
2. The amyloid-beta binding antibody or antigen-binding fragment thereof of clause 1, wherein the antibody or the antigen-binding fragment thereof comprises:
   a) a Heavy Chain Variable Region (VH) comprising the sequence of SEQ ID NO: 10 or a Heavy Chain Variable Region (VH) having at least 96%, 97%, 98%, or 99%, sequence identity to the amino acid sequence of SEQ ID NO: 10; or
   b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99%, sequence identity to the amino acid sequence of SEQ ID NO: 30; or
   c) a Heavy Chain Variable Region (VH) comprising the sequence of SEQ ID NO: 40 or a Heavy Chain Variable Region (VH) having at least 93%, 94%, 95%, 96%, 97%, 98%, or 99%, sequence identity to the amino acid sequence of SEQ ID NO: 40.
3. The amyloid-beta binding antibody or antigen-binding fragment thereof of any of the preceding clauses, wherein the antibody or the antigen-binding fragment thereof comprises:
   a) a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14; or
   b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; or
   c) a Heavy Chain Variable Region (VH) comprising the sequence of SEQ ID NO: 40 or a Heavy Chain Variable Region (VH) having at least 93%, 94%, 95%, 96%, 97%, 98%, or 99% sequence identity to the amino acid sequence of SEQ ID NO: 40; and a Light Chain Variable Region (VL) comprising the sequence of SEQ ID NO: 44 or a Light Chain Variable Region (VL) having at least 97%, 98%, or 99% sequence identity to the amino acid sequence of SEQ ID NO: 44.
4. The amyloid-beta binding antibody or antigen-binding fragment thereof of any of the preceding clauses, wherein the antibody or the antigen-binding fragment thereof comprises:
   a) a Heavy Chain Variable Region (VH) comprising the sequence of SEQ ID NO: 10 and a Light Chain Variable Region (VL) comprising the sequence of SEQ ID NO: 14; or
   b) a Heavy Chain Variable Region (VH) comprising the sequence of SEQ ID NO: 30 and a Light Chain Variable Region (VL) comprising the sequence of SEQ ID NO: 34; or
   c) a Heavy Chain Variable Region (VH) comprising the sequence of SEQ ID NO: 40 and a Light Chain Variable Region (VL) comprising the sequence of SEQ ID NO: 44.
5. The amyloid-beta binding antibody or antigen-binding fragment thereof of any of the preceding clauses, wherein the antibody comprises:
   a) a Heavy Chain (HC) comprising the amino sequence of SEQ ID NO: 20 and a Light Chain (LC) comprising the amino sequence of SEQ ID NO: 21; or
   b) a Heavy Chain (HC) comprising the amino sequence of SEQ ID NO: 22 and a Light Chain (LC) comprising the amino sequence of SEQ ID NO:23; or
   c) a Heavy Chain (HC) comprising the amino sequence of SEQ ID NO: 24 and a Light Chain (LC) comprising the amino sequence of SEQ ID NO: 25; or
   d) a Heavy Chain (HC) comprising the amino sequence of SEQ ID NO: 27 and a Light Chain (LC) comprising the amino sequence of SEQ ID NO: 28; or
   e) a Heavy Chain (HC) comprising the amino sequence of SEQ ID NO: 38 and a Light Chain (LC) comprising the amino sequence of SEQ ID NO: 39; or
   f) a Heavy Chain (HC) comprising the amino sequence of SEQ ID NO: 48 and a Light Chain (LC) comprising the amino sequence of SEQ ID NO: 49; or
   g) a Heavy Chain (HC) comprising the amino sequence of SEQ ID NO: 50 and a Light Chain (LC) comprising the amino sequence of SEQ ID NO: 51.
6. The amyloid-beta binding antibody or antigen-binding fragment thereof of any of the preceding clauses, which binds to an epitope within amino acids residues 1-5 (SEQ ID NO: 7), 1-8 (SEQ ID NO: 2), 22-35 (SEQ ID NO: 29) or 26-34 (SEQ ID NO: 8) of SEQ ID NO: 1 or to an equivalent epitope in non-human amyloid-beta.
7. The amyloid-beta binding antibody or antigen-binding fragment thereof of any of the preceding clauses, which is a murine, a chimeric, a humanized or a human antibody or an antigen-binding fragment thereof.
8. The amyloid-beta binding antibody or antigen-binding fragment thereof of any one of the preceding clauses, which is an IgM, IgG1, IgG2, IgG2a, IgG2b, IgG3 or IgG4 antibody or antigen-binding fragment thereof.
9. The amyloid-beta binding antibody or antigen-binding fragment thereof of any one of the preceding clauses, which is conjugated to another molecule, in particular a detectable label.
10. The amyloid-beta binding antibody or antigen-binding fragment thereof of any of the preceding clauses, for use in the diagnosis of an amyloid-beta associated disease, disorder or condition in a subject.
11. The amyloid-beta binding antibody or antigen-binding fragment thereof of any of the preceding clauses, for use in the detection of amyloid-beta in a biological sample.
12. The amyloid-beta binding antibody or antigen-binding fragment thereof of clause 11 wherein the biological sample is a body fluid sample or a buffer solution.
13. The amyloid-beta binding antibody or antigen-binding fragment thereof of any of clause 1 to 12, for use in the detection of amyloid-beta in a body fluid sample, wherein the body fluid sample is saliva, urine, nasal secretion, blood (including whole blood, plasma and serum, preferably plasma), brain and/or CSF sample, brain and/or ISF sample, more particularly blood, brain, CSF and/or ISF sample.
14. The amyloid-beta binding antibody or antigen-binding fragment thereof of any of the preceding clauses, for use in the diagnosis of an amyloid-beta associated disease, disorder or condition wherein the amyloid-beta associated disease, disorder or condition is selected from the group consisting of Alzheimer's Disease (AD), mild cognitive impairment (MCI), Down syndrome (DS), Down syndrome-related Alzheimer's Disease, cardiac amyloidosis, cerebral amyloid angiopathy (CAA), multiple sclerosis, Parkinson's disease, Parkinson's Disease with Dementia (PDD), Lewy body dementia, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure.
15. The amyloid-beta binding antibody or antigen-binding fragment thereof of any of the preceding clauses, for use in the diagnosis of an amyloid-beta associated disease, disorder or condition wherein the amyloid-beta associated disease, disorder or condition is Alzheimer's Disease (AD), Down syndrome (DS), Down syndrome-related Alzheimer's Disease, cerebral amyloid angiopathy (CAA), Myotonic dystrophy, or Lewy body dementia.
16. The amyloid-beta binding antibody or antigen-binding fragment thereof of any of the preceding clauses, for use in the diagnosis of an amyloid-beta associated disease, disorder or condition wherein the amyloid-beta associated disease, disorder or condition is Alzheimer's Disease (AD).
17. The amyloid-beta binding antibody or antigen-binding fragment thereof of any of the clauses 1 to 15 for use in the diagnosis of an amyloid-beta associated disease, disorder or condition wherein the amyloid-beta associated disease, disorder or condition is Down Syndrome (DS).
18. The amyloid-beta binding antibody or antigen-binding fragment thereof of any of the clauses 1 to 15 for use in the diagnosis of an amyloid-beta associated disease, disorder or condition wherein the amyloid-beta associated disease, disorder or condition is Down Syndrome-related Alzheimer's disease.
19. A method of detecting amyloid-beta in a sample obtained from a subject, the method comprising contacting the sample with the amyloid-beta binding antibody or antigen-binding fragment thereof of any of the preceding clauses and detecting binding of the antibody or antigen-binding fragment thereof in order to detect amyloid-beta in the sample.
20. A method of quantifying amyloid-beta in a sample obtained from a subject, the method comprising contacting the sample with the amyloid-beta binding antibody or antigen-binding fragment thereof of any of clauses 1-18 and quantifying amyloid-beta in a sample based on the level of binding of the antibody or antigen-binding fragment thereof to amyloid-beta.
21. A method for diagnosing a disease, disorder and/or condition associated with amyloid-beta comprising performing the method of clauses 19 or 20 wherein higher levels of amyloid-beta in the sample compared with a control level based on healthy subjects are indicative of a disease, disorder and/or condition associated with amyloid-beta.
22. A method for diagnosing a disease, disorder and/or condition associated with amyloid-beta comprising performing the method of clause 19 or 20 wherein similar or higher levels of amyloid-beta in the sample compared with a diseased control level are indicative of a disease, disorder and/or condition associated with amyloid-beta.
23. A method for classifying a disease, disorder and/or condition associated with amyloid-beta comprising:
   a. performing the method of clause 21 and/or 22,
   b. classifying the disease, disorder and/or condition associated with amyloid-beta.
24. A method for monitoring a disease, disorder and/or condition associated with amyloid-beta at two or more time points using samples from a subject comprising contacting the samples with an amyloid-beta binding antibody or antigen-binding fragment thereof of any of clauses 1-18, wherein;
   a. higher levels of amyloid-beta in the later sample compared with one or more earlier samples are indicative of progression of a disease, disorder and/or condition associated with amyloid-beta;
   b. lower levels of amyloid-beta in the later sample compared with one or more earlier samples are indicative of regression of a disease, disorder and/or condition associated with amyloid-beta; and/or
   c. no significant change of levels of amyloid-beta in the later sample compared with one or more earlier samples are indicative of lack of progression of a disease, disorder and/or condition associated with amyloid-beta.
25. A method for selecting a therapy for treatment of a disease, disorder and/or condition associated with amyloid-beta comprising contacting samples taken before and after treatment with the therapy with an amyloid-beta binding antibody or antigen-binding fragment thereof of any of clauses 1-18, wherein;
   a. lower levels of amyloid-beta in the sample taken after treatment compared with the sample taken before treatment are indicative of successful treatment of a disease, disorder and/or condition associated with amyloid-beta and thus the therapy is selected for treatment;
   b. no significant change of levels of amyloid-beta in the sample taken after treatment compared with the sample taken before treatment are indicative of successful treatment of a disease, disorder and/or condition associated with amyloid-beta and thus the therapy is selected for treatment;
   c. a decline in the rate of increase of levels of amyloid-beta between samples taken during treatment compared with samples taken before treatment are indicative of successful treatment of a disease, disorder and/or condition associated with amyloid-beta and thus the therapy is selected for treatment;
   d. higher levels of amyloid-beta in the sample taken after treatment compared with the sample taken before treatment are indicative of unsuccessful treatment of a disease, disorder and/or condition associated with amyloid-beta and thus the therapy is not selected for treatment; or
   e. no decline in the rate of increase of levels of amyloid-beta between samples taken during treatment compared with samples taken before treatment are indicative of unsuccessful treatment of a disease, disorder and/or condition associated with amyloid-beta and thus the therapy is not selected for treatment.
26. A method for assessing a candidate therapy for a disease, disorder and/or condition associated with amyloid-beta, the method comprising, following treatment of one or more subjects, contacting samples from the one or more treated subjects with an antibody or antigen-binding fragment of any of clauses 1-18, wherein lower levels of amyloid-beta in the samples compared with levels in corresponding samples from subjects not treated with the therapy are indicative of successful treatment of a disease, disorder and/or condition associated with amyloid-beta.
27. The method of clause 26 performed at multiple time points in matched samples between the treatment and placebo groups in order to monitor the effectiveness of the candidate therapy over a defined time period.
28. The method of clause 26 or 27 which comprises contacting samples from the one or more treated subjects and the subjects not treated with the therapy with an antibody or antigen-binding fragment of any of clauses 1-18 prior to treatment, with the therapy or placebo respectively, to determine base levels of amyloid-beta.
29. The method according to any one of clauses 19 to 28 wherein the disease, disorder and/or condition associated with amyloid-beta is selected from the group consisting of Alzheimer's Disease (AD), mild cognitive impairment (MCI), Down syndrome (DS), Down syndrome-related Alzheimer's Disease, cardiac amyloidosis, cerebral amyloid angiopathy (CAA), multiple sclerosis, Parkinson's disease, Parkinson's Disease with Dementia (PDD), Lewy body dementia, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure.
30. The method according to clause 29 wherein the amyloid-beta associated disease, disorder or condition is Alzheimer's Disease (AD), Down syndrome (DS), Down syndrome-related Alzheimer's Disease, cerebral amyloid angiopathy (CAA), Myotonic dystrophy, or Lewy body dementia.
31. The method according to clause 29 or 30 wherein the amyloid-beta associated disease, disorder or condition is Alzheimer's Disease (AD).
32. The method according to clause 29 or 30 wherein the amyloid-beta associated disease, disorder or condition is Down Syndrome (DS).
33. A diagnostic composition comprising the amyloid-beta binding antibody or antigen-binding fragment thereof of any one of clauses 1 to 18 and an acceptable carrier and/or excipient.
34. A nucleic acid encoding the amyloid-beta binding antibody or antigen-binding fragment thereof of any one of clauses 1 to 18.
35. A nucleic acid comprising a nucleotide sequence as provided in SEQ ID NO: 18, SEQ ID NO: 19, SEQ ID NO: 52, SEQ ID NO: 53, SEQ ID NO: 54 or SEQ ID NO: 55.
36. A recombinant vector comprising the nucleic acid of clause 34 or 35.
37. A host cell comprising the nucleic acid of clause 34 or 35 and/or the vector of clause 36.
38. An isolated host cell that expresses the amyloid-beta binding antibody or antigen-binding fragment of any one of clauses 1 to 18.
39. A method for producing an amyloid-beta binding antibody or antigen-binding fragment thereof, comprising the steps of:
   a. culturing the host cell of clause 37 or 38 under conditions suitable for producing the amyloid-beta binding antibody or antigen-binding fragment thereof, and
   b. recovering the amyloid-beta binding antibody or antigen-binding fragment thereof.
40. A kit for diagnosis of a disease, disorder or condition associated with amyloid-beta, or a kit for use in a method of any one of clauses 19 to 32, comprising the amyloid-beta binding antibody or antigen-binding fragment thereof according to any one of clauses 1 to 18.
41. The amyloid-beta binding antibody or antigen-binding fragment thereof of any one of clauses 1-18 for research use, in particular as an analytical tool or reference molecule.
42. The amyloid-beta binding antibody or antigen-binding fragment thereof of any one of clauses 1-18 for use in detecting amyloid-beta aggregates, including plaques, in vitro or in vivo.
43. The amyloid-beta binding antibody or antigen-binding fragment thereof for use of clause 42 which is used for histochemical detection in brain tissue.

### BRIEF DESCRIPTION OF FIGURES AND TABLES

FIG. 1. ACI-24-41F12-Ab3 (murine IgG2a isotype) binds Abeta1-42 with a Kd of 66 pM. No binding was observed to BSA protein. Data are expressed as O.D., and shown as mean±SD of a single assay duplicate.

FIG. 2. Antibody ACI-31-25B1-Ab2 (murine IgG2a isotype) binds to Abeta1-42 with a Kd of 41 pM. No binding was observed to BSA protein. Data are expressed as O.D.

FIG. 3. Antibody ACI-31-30C11-Ab1 (murine IgG2a isotype) binds to Abeta1-42 with a Kd of 196 pM. No binding was observed to BSA protein. Data are expressed as O.D.

FIG. 4. Antibody ACI-31-25B1-Ab2 (murine IgG2a isotype) captures soluble Abeta1-42 with a Kd of 312 pM. Data are expressed as O.D.

FIG. 5. Antibody ACI-8041-1F11B5-Ab1 (a murine IgG2a isotype) captures soluble Abeta1-42 with a Kd of 343 pM. Data are expressed as O.D.

FIG. 6. Antibody ACI-8041-9D7E1-Ab1 (a murine IgG2a isotype) captures soluble Abeta1-42 with a Kd of 1.1 nM. Data are expressed as O.D.

FIG. 7. Antibody ACI-8041-10H3B11-Ab1 (a murine IgG2a isotype) captures soluble Abeta1-42 with a Kd of 2.6 nM. Data are expressed as O.D.

FIG. 8. A) An indirect ELISA comparing the binding of individual antibodies ACI-24-41F12-Ab3 (a murine IgG2a isotype) and ACI-31-25B1-Ab2 (a murine IgG2a/lambda isotype) to sAPPalpha, normalized to Abeta1-42 binding. Scrambled Abeta1-42 was used as a control, showing no binding. Antibody ACI-24-41F12-Ab3 shows binding to sAPPalpha whereas no binding is observed for antibody ACI-31-25B1-Ab2. B) A sandwich pairing of antibodies ACI-24-41F12-Ab3 (detecting) and ACI-31-25B1-Ab2 (capturing) detects soluble Abeta1-42 with a Kd of 9.7 nM with selectivity for Abeta1-42 over scrambled Abeta1-42 or sAPPalpha. Data are expressed as O.D. or as % of Abeta1-42 binding.

Table 1. Abeta1-42 binding regions and epitopes, and interaction with sAPPalpha.

Table 2. Nucleotide sequences of the Heavy Chain and Light Chain Variable domains (VH and VL).

Table 3. Amino acid sequences of the Heavy Chain and Light Chain Variable domains (VH and VL).

Table 4. Amino acid sequences of Heavy Chain (HC) and Light Chain (LC) variable and constant regions.

Table 5. Amino acid sequences of Heavy Chain (HC) and Light Chain (LC) constant regions.

The invention is further described with reference to the following non-limiting examples:

### Methods Example 1. Antibody Generation

1.1. Preparation of an Abeta Liposomal Vaccine Composition

The liposome-based antigenic constructs were prepared according to the protocols published in WO2012/055933. The liposomal vaccine with tetrapalmitoylated human Abeta1-15 peptide as antigen was used for generating antibodies ACI-24-41F12-Ab2 (murine IgG2b) and ACI-24-41F12-Ab3 (murine IgG2a isotype and a chimeric mouse/human IgG1 isotype), or with tetrapalmitoylated human Abeta22-35 peptide as antigen for generating ACI-31-25B1-Ab2 (murine IgG2b/lambda, murine IgG2a/lambda and a chimeric mouse/human IgG1 isotype),) and ACI-31-30C11-Ab1 (murine IgG2b), or with tetrapalmitoylated human Abeta1-23 peptide as antigen for generating ACI-8037-103H5-Ab2 (murine IgG2a) and ACI-8037-109F4-Ab1 (murine IgG2a), or with tetrapalmitoylated human Abeta5-23 peptide as antigen for generating ACI-8039-306H5-Ab1 (murine IgG2a), ACI-8039-307F7-Ab2 (murine IgG2a), and ACI-8039-309D9-Ab2 (murine IgG2a), or with tetrapalmitoylated human Abeta10-29 peptide as antigen for generating ACI-8041-1F11B5-Ab1 (murine IgG2a), ACI-8041-2B9H5-Ab1 (murine IgG2a), ACI-8041-3G11B3-Ab1 (murine IgG2a), ACI-8041-4G6D2-Ab1 (murine IgG2a), ACI-8041-5A2D4-Ab1 (murine IgG2a), ACI-8041-5A6H9-Ab1 (murine IgG2a), ACI-8041-6C3C4-Ab1 (murine IgG2a), ACI-8041-9D7E1-Ab1 (murine IgG2a), and ACI-8041-10H3B11-Ab1 (murine IgG2a). Mice (C57BL/6, 9 weeks old, female) were injected with the vaccine by subcutaneous (s.c.) injections on three occasions with a two-week interval between each immunization (at days 0, 14, and 28). Blood samples were collected and plasma were prepared 7 days before the first immunization and seven days after each immunization (at days 7, 21, and 35). After the last bleeding, mice were selected based on vaccine response and splenocytes used for fusions with mouse myeloma cells, 65 days after study initiation. Hybridomas binding to the target were selected, sub-cloned to obtain monoclonal antibodies, and then ranked for target-binding properties to select hybridoma clones for sequencing and recombinant antibody production. The data below describe the properties of ACI-24-41F12-Ab2, a hybridoma-derived murine IgG2b/kappa antibody, and a recombinant murine IgG2a/kappa antibody (hereafter referred as ACI-24-41F12-Ab3 (IgG2a isotype)) and a recombinant chimeric mouse/human IgG1 isotype (hereafter referred as ACI-24-41F12-Ab3 (hIgG1 isotype)) having the same variable heavy (VH) and light (VL) chain sequences, and binding properties (data not shown), as the ACI-24-41F12-Ab2 antibody. The data below further describe some properties of ACI-31-25B1-Ab2 in the form of a murine IgG2a/lambda, and ACI-31-30C11-Ab1, ACI-8037-103H5-Ab2, ACI-8037-109F4-Ab1, ACI-8039-306H5-Ab1, ACI-8039-307F7-Ab2, ACI-8039-309D9-Ab2, ACI-8041-1F11B5-Ab1, ACI-8041-2B9H5-Ab1, ACI-8041-3G11B3-Ab1, ACI-8041-4G6D2-Ab1, ACI-8041-5A2D4-Ab1, ACI-8041-5A6H9-Ab1, ACI-8041-6C3C4-Ab1, ACI-8041-9D7E1-Ab1, and ACI-8041-10H3B11-Ab1, with a murine IgG2a isotype constant region heavy chain and kappa constant region light chain (Table 5).

### Example 2. Preparation of Abeta1-42 for Assays

Abeta1-42 lyophilized powder (Bachem) was reconstituted in hexafluoroisopropanol (HFIP, Sigma) to 1 mM. The peptide solution was sonicated for 15 min at room temperature, agitated overnight, and aliquots made into non-siliconized microcentrifuge tubes. The HFIP was then evaporated under a stream of argon. The resulting peptide film was vacuum dried for 10 min and stored at −80° C. until use. Alternatively, to assay for target capture in solution, a biotinylated version of the Abeta1-42 peptide was used. This was prepared in a similar manner to that described above.

### Example 3. Target Binding

Antibody target binding was evaluated by an enzyme-linked immunosorbent assay (ELISA). Briefly, Nunc MaxiSorp 96-well plates (Nunc) were coated for 18 h at 4° C. with 50 μL of 10 pg/mL Abeta1-42 or bovine serum albumin (BSA, Sigma) diluted in PBS. In selected assays, the full-length sAPPalpha fragment (Sigma) was additionally included by coating wells as described above, but at 4 pg/mL (0.04 μM). Plates were then washed 4-times with 300 μL of PBS containing 0.05% Tween20 (Millipore). To block non-specific binding to the plate, freshly prepared solution of PBS containing 0.05% Tween20 and 1% BSA, was added to each well (100 μL/well) and the plates were incubated for 1 h at 37° C. Primary antibodies were diluted to 100 nM in PBS containing 1% BSA and 0.05% Tween20, followed by 3-fold serial dilutions in duplicates. Solutions were added to plates at 50 μL/well and incubated for 1 h at 37° C. Subsequently, the plates were washed 4-times with 300 μL of PBS containing 0.05% Tween20. Plates were then incubated with horseradish peroxidase (HRP) conjugated goat anti-mouse secondary IgG (Abcam) for 1 h at 37° C. After final wash step (4-times with 300 μL of PBS containing 0.05% Tween20), plates were incubated with 3,3′,5,5′-tetramethylbenzidine (TMB) substrate (BD Biosciences) and reaction stopped with a Stop Solution (Sigma). Finally, optical densities (O.D.) were read at 450 nm absorption wavelength using a Tecan Infinite plate reader. Measured values are expressed as mean O.D. from a duplicate run ±1 standard deviation of the mean (S.D.).

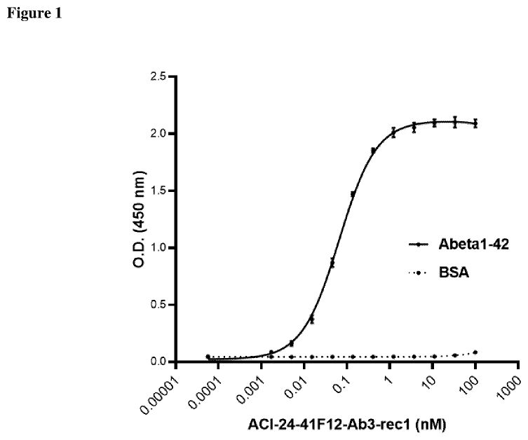

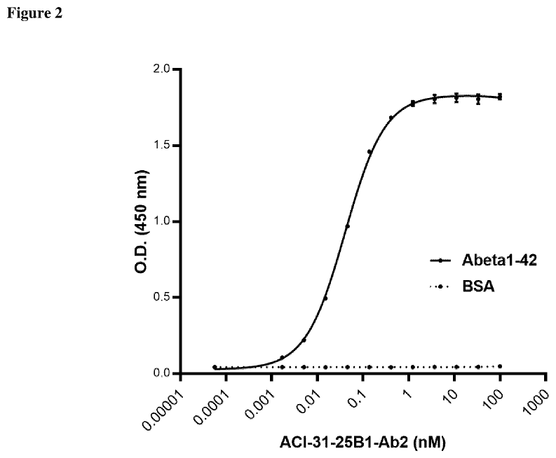

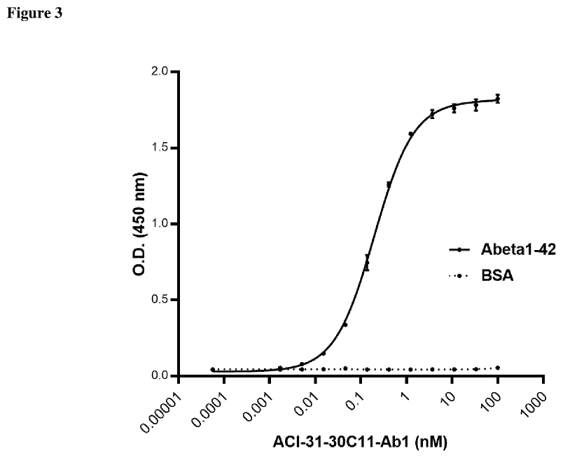

An ELISA assay was used to evaluate binding of antibody ACI-24-41F12-Ab3 (a recombinant murine IgG2a isotype, also known as ACI-24-41F12-Ab3-rec1), antibody ACI-31-25B1-Ab2 (a murine IgG2a/lambda isotype), and antibody ACI-31-30C11-Ab1 (a murine IgG2a isotype) to Abeta1-42. Antibody ACI-24-41F12-Ab3 bound to Abeta1-42 with a Kd of 66 pM, and no cross-reactivity to BSA at identical protein concentration was observed (FIG. 1). The assay was repeated at least four independent times with comparable results. Antibody ACI-31-25B1-Ab2 bound to Abeta1-42 with a Kd of 41 pM (FIG. 2), and antibody ACI-31-30C11-Ab1 with a Kd of 196 pM (FIG. 3), with no cross-reactivity to BSA.

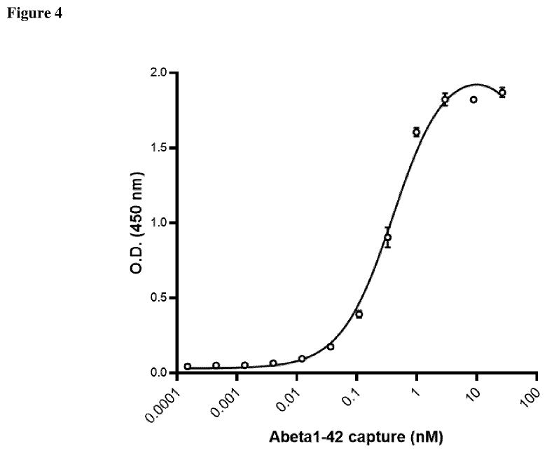

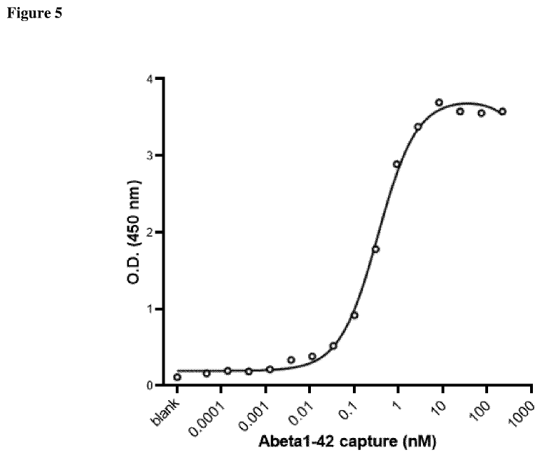

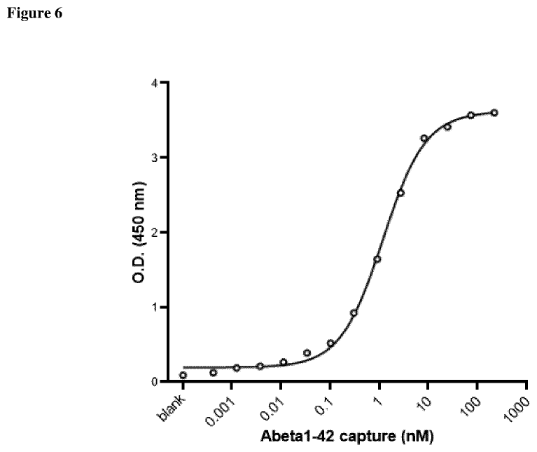

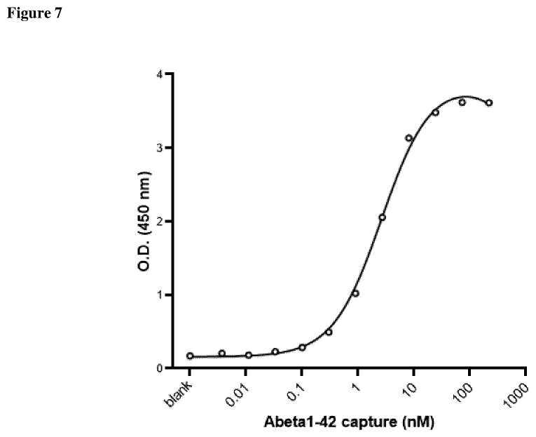

A target capture assay by ELISA was used to further evaluate binding of antibodies ACI-31-25B1-Ab2, ACI-8041-1F11B5-Ab1, ACI-8041-9D7E1-Ab1 and ACI-8041-10H3B11-Ab1 to Abeta1-42 in solution as an indication of soluble target capture in biological fluids. Briefly, a Nunc MaxiSorp 96-well plate (Nunc) was coated for 18 h at 4° C. with 50 μL of 5 pg/mL goat anti-mouse IgG, Fc-gamma fragment specific antibody (Jackson Immune Research) diluted in carbonate-bicarbonate buffer. The plate was then washed 4-times with 300 μL of PBS containing 0.05% Tween20 (Millipore). To block non-specific binding to the plate, freshly prepared solution of PBS containing 0.05% Tween20 and 1% BSA, was added to each well (100 μL/well) and the plate was incubated for 1 h at 37° C. Primary antibody was diluted at 5 μg/mL (33.3 nM) in PBS containing 1% BSA and 0.05% Tween20, 50 μL/well was added to each well and the plate was incubated for 1 h at 37° C. Subsequently, the plate was washed 4-times with 300 μL of PBS containing 0.05% Tween20. Human Abeta1-42 biotinylated at the peptide C-terminus was diluted to 10 μg/mL (2.2 μM) in PBS containing 1% BSA and 0.05% Tween20, followed by 3-fold serial dilutions, 50 μL was added to each well and the plate was incubated for 1 h at 37° C. After washing 4-times with 300 μL of PBS containing 0.05% Tween20, the plate was incubated for 45 min at ambient temperature with 50 μL/well of streptavidin-HRP (R&D systems) diluted 1:200 in PBS containing 1% BSA and 0.05% Tween20. The plate was again washed 4-times with 300 μL of PBS containing 0.05% Tween20 before incubating at ambient temperature for 20 or 30 min with 50 or 100 μL/well of 3,3′,5,5′-tetramethylbenzidine (TMB) enzyme substrate (BD Bioscience). Reaction was stopped with a 1.2 M HCl (Sigma) solution. Finally, optical densities (O.D.) were read at 450 nm absorption wavelength using a Tecan Infinite plate reader. Measured values are expressed as O.D. Antibody ACI-31-25B1-Ab2 captured Abeta1-42 in solution with a Kd of 312 pM (FIG. 4), antibody ACI-8041-1F11B5-Ab1 captured Abeta1-42 in solution with a Kd of 343 pM (FIG. 5), antibody ACI-8041-9D7E1-Ab1 captured Abeta1-42 in solution with a Kd of 1.1 nM (FIG. 6), and antibody ACI-8041-10H3B11-Ab1 captured Abeta1-42 in solution with a Kd of 2.6 nM (FIG. 7). Antibodies ACI-24-41F12-Ab3, ACI-31-30C11-Ab1, ACI-8037-103H5-Ab2, ACI-8037-109F4-Ab1, ACI-8039-306H5-Ab1, ACI-8039-307F7-Ab2, ACI-8039-309D9-Ab2, ACI-8041-2B9H5-Ab1, ACI-8041-3G11B3-Ab1, ACI-8041-4G6D2-Ab1, ACI-8041-5A2D4-Ab1, ACI-8041-5A6H9-Ab1, and ACI-8041-6C3C4-Ab1 also captured Abeta1-42 in solution (data not shown).

Furthermore, selected antibodies were assayed to verify binding and selectivity for Abeta1-42 by using a pairing or sandwich ELISA. Briefly, a Nunc MaxiSorp 96-well plate (Nunc) was coated for 18 h at 4° C. with 50 μL of capture antibody at 5 μg/mL, diluted in carbonate-bicarbonate buffer. The plate was then washed and blocked as described in the preceding paragraph. Subsequently, analyte proteins were diluted to 666 nM for Abeta1-42 (Bachem) and scrambled Abeta1-42 (Anaspec), or 165 nM for sAPPalpha (Sigma) in PBS containing 1% BSA and 0.05% Tween20, followed by 3-fold serial dilutions and 50 μL added to each well. The plate was incubated for 2 h at 37° C. and then washed as previously described and then incubated for 1 h at 37° C. with 50 μL/well of biotinylated detection antibody, diluted to 2 μg/mL in PBS containing 1% BSA and 0.05% Tween20. The subsequent steps are the same as described in the preceding paragraph for Abeta1-42 target capture, except that incubation with TMB was only done with 100 μL/well for 30 min.

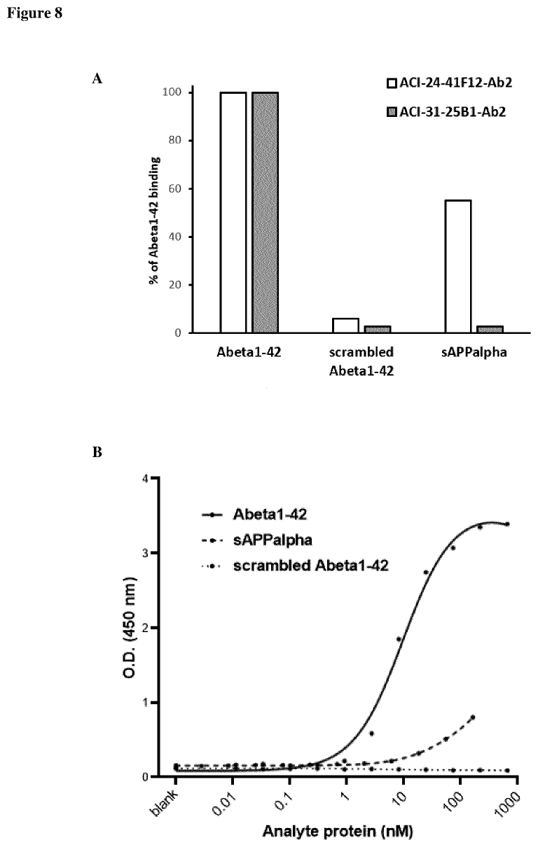

Sandwich ELISAs run with selected antibody pairs detected Abeta1-42 in solution with Kds ranging from 100 pM. Results for an example of a sandwich ELISA using antibody ACI-24-41F12-Ab3, an N-terminus Abeta1-42 binder that cross-reacts with sAPPalpha, and one mid-domain binder that does not bind sAPPalpha are shown in FIG. 8A. This demonstrates assay selectivity for soluble Abeta1-42 over sAPPalpha and a Kd of 9.7 nM for soluble Abeta1-42 (FIG. 8B). The sandwich pairing of antibodies ACI-24-41F12-Ab3 (detecting) and ACI-31-25B1-Ab2 (capturing) detects soluble Abeta1-42. Additionally, selectivity for soluble Abeta1-42 was further substantiated with total lack of signal when using scrambled Abeta1-42 as the assay analyte, consisting of all the same 42 amino acids present in the human Abeta1-42 peptide.

### Example 4. Epitope Mapping

Epitope mapping of ACI-24-41F12-Ab2, ACI-31-25B1-Ab2, and ACI-31-30C11-Ab1 was performed by ELISA using a peptide library spanning the amino acid sequence of Abeta1-42 consisting of 33 overlapping biotinylated peptides of 8, 9, or 10 residues each (Mimotopes Ltd., Melbourne, Australia). Biotinylated Abeta1-42 (Bachem) was used as a control peptide. Epitope mapping ELISA was done according to the manufacturer's instructions. Briefly, streptavidin-coated plates (Nunc) were blocked with 0.1% BSA in PBS for 18 hours at 4° C. on a horizontal shaker at 50 rpm. After washing with PBS containing 0.05% Tween20, plates were coated with each peptide from the library, diluted to the final concentration of 10 μM in 0.1% BSA containing 0.1% sodium azide in PBS for 1 hour at ambient temperature. After washing, plates were incubated for 1 hour at ambient temperature with ACI-24-41F12-Ab2, ACI-31-25B1-Ab2, or ACI-31-30C11-Ab1 at 1 μg/mL diluted in 2% BSA containing 0.1% sodium azide in PBS. Plates were washed again and incubated with alkaline phosphatase (AP) conjugated goat anti-mouse IgG (Jackson Immune Research) for 1 hour at ambient temperature. After final wash, plates were incubated for 2 hours with phosphatase substrate (pNPP, Sigma) and absorbance was read at 405 nm using a plate reader (Tecan).

To determine the epitope of ACI-24-41F12-Ab2 and ACI-31-25B1-Ab2, on Abeta1-42, binding of the antibodies to 33 overlapping 8, 9, or 10-mer peptides covering the entire sequence of Abeta1-42 was analysed by ELISA using streptavidin plates and peptides labelled with a terminal biotin (Table 1). In the cases where epitope was not mapped, the binding region was determined using the same assay as described above, but with target peptides corresponding to the different vaccine antigen sequences used to generate the antibodies. Binding to the full length Abeta1-42 was used as positive control. The epitope of ACI-24-41F12-Ab2 mapped to the N-terminus region and was identified to be within residues number 1 to 8 (more precisely within amino acids residues 1-5, wherein amino acids 1 and 2 of Abeta 1-42 are essential to antibody binding) of the Abeta1-42 peptide sequence. The epitope of ACI-31-25B1-Ab2 was mapped to the mid-domain region and identified to be within residues 22-35, more precisely within amino acids residues 26-34 of the Abeta1-42 peptide sequence (Table 1). The Abeta1-42 binding regions were Abeta1-5 for ACI-8037-103H5-Ab2 and ACI-8037-109F4-Ab1, Abeta17-23 for ACI-8039-306H5-Ab1, ACI-8039-307F7-Ab2, ACI-8039-309D9-Ab2, ACI-8041-1F111B5-Ab1, ACI-8041-2B9H5-Ab1, ACI-8041-3G11B3-Ab1, ACI-8041-4G6D2-Ab1, ACI-8041-5A2D4-Ab1, ACI-8041-5A61H9-Ab1, ACI-8041-6C3C4-Ab1, ACI-8041-9D7E1-Ab1, ACI-8041-11B3B11-Ab1, and Abeta22-35 for ACI-31-30C11-Ab1. Cross-reactivity to sAPPalpha, that shares the same amino acid sequence with human Abeta1-42 between residues 1 and 16 is additionally shown in Table 1. Cross-reactivity was determined for antibodies AC-24-41F12-Ab2, ACI-8037-103H5-Ab2, and ACI-8037-109F4-Ab1. No cross-reactivity was determined for antibodies AC-31-25B1-Ab2, AC-31-30C11-Ab1, AC-8039-306H5-Ab1, AC-8039-307F7-Ab2, AC-8039-309D9-Ab2, ACI-8041- F1B5-Ab1, AC-8041-2B95-Ab1, ACD-8041-3G11B3-Ab1, ACI-8041-4G6D2-Ab1, ACI-8041-5A2D4-Ab1, AC-8041-5A619-Ab1, ACD-8041-63C4-Ab1, ACI-8041-9D7E1-Ab1 and AC-8041-13B11-Ab.

TABLE 1
Abetal-42 binding regions and epitopes, and interaction with sAPPalpha:

| | Binding regions, aa | Epitopes, aa | sAPPalpha binding |
|---|---|---|---|
| ACI-24-41F12-Ab2 and | 1-8 (SEQID NO: 2) | 1-5 (SEQ ID NO: 7) | + |
| ACI-24-41F12-Ab3 | | | |
| ACI-31-25B1-Ab2 | 22-35 (SEQ ID NO: 29) | 26-34 (SEQ ID NO: 8) | — |
| ACI-31-30C11-Ab1 | 22-35 (SEQ ID NO: 29) | ND | — |
| ACI-8037-103H5-Ab2 | 1-5 (SEQ ID NO: 7) | ND | +++ |
| ACI-8037-109F4-Ab1 | 1-5 (SEQ ID NO: 7) | ND | +++ |
| ACI-8039-306H5-Ab1 | 17-23 (SEQ ID NO: 9) | ND | — |
| ACI-8039-307F7-Ab2 | 17-23 (SEQ ID NO: 9) | ND | — |
| ACI-8039-309D9-Ab2 | 17-23 (SEQ ID NO: 9) | ND | — |
| ACI-8041-1F11B5-Ab1 | 17-23 (SEQ ID NO: 9) | ND | — |
| ACI-8041-2B9H5-Ab1 | 17-23 (SEQ ID NO: 9) | ND | — |
| ACI-8041-3G11B3-Ab1 | 17-23 (SEQ ID NO: 9) | ND | — |
| ACI-8041-4G6D2-Ab1 | 17-23 (SEQ ID NO: 9) | ND | — |
| ACI-8041-5A2D4-Ab1 | 17-23 (SEQ ID NO: 9) | ND | — |
| ACI-8041-5A6H9-Ab1 | 17-23 (SEQ ID NO: 9) | ND | — |
| ACI-8041-6C3C4-Ab1 | 17-23 (SEQ ID NO: 9) | ND | — |
| ACI-8041-9D7E1-Ab1 | 17-23 (SEQ ID NO: 9) | ND | — |
| ACI-8041-10H3B11-Ab1 | 17-23 (SEQ ID NO: 9) | ND | — |

ND: not determined,
—: no binding,
+: low binding,
+++: high binding

### Example 5. Antibody Variable Region Gene Sequencing

Mouse hybridoma cells were harvested and lysed using a lysis buffer containing guanidinium salts that deactivate RNases. The genomic DNA was then eliminated by adding RNase-free DNase to the sample. After cell disruption and elimination of genomic DNA, the RNA was purified to a silica-based affinity column using multiple washes and eluted from the column using RNase-free water. Once RNA was extracted, its purity and concentration was measured spectrophotometrically. The integrity of the RNA was assessed on a denaturing agarose gel and RNA was reverse transcribed into cDNA using reverse transcriptase (RT). Before adding the reaction mixture, the RNA was heated to 70° C. for 10 min to disrupt RNA secondary structures. The RT products were directly used for PCR amplification. For high-fidelity PCR amplification of the cDNA, each of the variable region primers corresponding to the different gene families encoding for antibodies were individually mixed with the constant primer, for variable heavy chain domain (VH) and variable light chain domain (VL) separately. Initially, a degenerate primer pool was used (12 for VH and 12 for VL) and, depending on results, a second pool was used to obtain PCR products. After the PCR reaction, the products were analysed by gel electrophoresis on 2% agarose gels stained with ethidium bromide. The PCR products for VL and VH were individually purified on an agarose gel using tris-acetate-EDTA (TAE). The purified fragments excised from the gel were then sequenced using the dye-terminator sequencing method. The same primers as those used for the PCR were used for the sequencing reaction. Sequencing was carried out in both directions to provide overlap at both ends. Sequencing data were analysed on the Ig Blast/Kabat database. Nucleotide sequences for VH and VL are shown in Table 2. Protein sequences for VH and VL, and their complementarity-determining regions (CDRs) are shown in Table 3. Protein sequences for HC and LC of mouse IgG2b, mouse IgG2a, and mouse/human chimeric IgG1 are shown in Table 4.

TABLE 2
Nucleotide sequences of the Heavy Chain and Light Chain Variable
domains (VH and VL)

| Antibody | VH | VL |
|---|---|---|
| ACI-24- | CAGGTTACTCTGAAAGAGTCTGGCCCTGG | GATGTTTTGATGACCCAAACTCCACTCTC |
| 41F12- | GATATTGCAGTCCTCCCAGACCCTCAGTC | CCTGCCTGTCAGTCTTGGAGATCAAGCCT |
| Ab2 and | TGACTTGTTCTTTCTCTGGGTTTTCACTGA | CCATCTCTTGCAGATCTAGTCAGAGCATT |
| ACI-24- | GCACTTCTGGTATGGGTGTGAGCTGGATT | GTACATAGTAATGGAAACACCTATTTAG |
| 41F12- | CGTCAGCCTTCAGGAAAGGGTCTGGAGT | AATGGTACCTGCAGAAACCAGGCCAGTC |
| Ab3 | GGCTGGCACACATTTACTGGGATGATGA | TCCAAAGCTCCTGATCTACAAAGTTTCCA |
| (IgG2a | CAAGCGCTATAACCCATCCCTGAAGAGC | ACCGATTTTCTGGGGTCCCAGACAGGTTC |
| isotype) | CGGCTCACAATCTCCAAGGATACCTCCAG | AGTGGCAGTGGATCAGGGACAGATTTCA |
| and | AAACCAGGTATTCCTCAAGATCACCAGT | CACTCAAGATCAGCAGAGTGGAGGCTGA |
| ACI-24- | GTGGACACTGCAGATACTGCCACATACT | GGATCTGGGAGTTTATTACTGCTTTCAAG |
| 41F12- | ACTGTGCTCGAAGGAGGAATGGTTACGA | GTTCACATGTTCCGCTCACGTTCGGTGCT |
| Ab3 | CGGGGGGTTTGCTTACTGGGGCCAAGGG | GGGACCAAGCTGGAGCTGAAA |
| (hIgG1 | ACTCTGGTCACTGTCTCTGCA | (SEQ ID NO: 19) |
| isotype) | (SEQ ID NO: 18) | |
| ACI-31- | GAGGTGCAGCTGGTGGAGTCTGGGGGAG | CAACTTGTGCTCACTCAGTCATCTTCAGC |
| 25B1-Ab2 | ACTTAGTGAAGCCTGGAGGGTCCCTGAA | CTCTTTCTCCCTGGGAGCCTCAGCAAAAC |
| (IgG2b | ACTCTCCTGTGCAGCCTCTAGATTCACTT | TCACGTGCACCTTGAGTAGTCAGCACATT |
| isotype | TCAGTACCTATGGCATGTCTTGGGTTCGC | ACGTACACCATTGAGTGGTATCAGCAAC |
| with a | CAGACTCCAGACAAGAGGCTGGAATGGG | AGCCACTCAAGCCTCCTAAGTATGTGATG |
| lambda | TCGCAACCATTAGTAGTGGTAGAAGTTAC | GTACTTGAGAAAGATGGAAGCCACAGCA |
| light chain | ACCTACTATGCAGACAGTGTGAAGGGGC | CAGGTGATGGGATTCCTGATCGCTTCTCT |
| and IgG2a | GATTCACCATCTCCAGAGACAATGCCAA | GGATCCAGCTCTGGTGCTGATCGTTACCT |
| isotype | GAACACCCTGTACCTGCAAATGAGCAGT | TAGCATTTCCAACATCCAGCCTGAAGATG |
| with a | CTGAAGTCTGAGGACACAGCCATGTATT | AAGCAGTATACATCTGTGGTGTGGGTGAT |
| lambda | ACTGTGCAAGAAATAGTAACAACCAGAG | GCAATTAAGGAACAATTTGTGTATGTTTT |
| light chain) | GGATTACTATGCTATGGACTACTGGGGTC | CGGCGGTGGAACCAAGGTCACTGTCCTA |
| | AAGGAACCTCAGTCACCGTCTCCTCA | (SEQ ID NO: 53) |
| | (SEQ ID NO: 52) | |
| ACI-31- | CAGGTTCAGCTGCAGCAGTCTGGACCTG | GACATTGTGCTGACCCAATCTCCAGCTTC |
| 30C11- | AGCTGGTGAAGCCTGGGGCCTCAGTGAA | TTTGGCTGTGTCTCTAGGGCAGAGGGCCA |
| Ab1 (two | GATTTCCTGCAAGGCTTCTGGCTACGCAT | CCATATCCTGCAGAGCCAGTAAAAGTGTT |
| isotypes: | TCAGTAGCTCCTGGCTGAACTGGGTGAA | GATAATTATGGCAAAAGTTTTATGCACTG |
| IgG2b with | GCAGAGGCCTGGAGAGGGTCTTGAGTGG | GTTCCAGCAGAAACCAGGACAGCCACCC |
| a kappa | ATTGGACGGATTTATCCTGGAGATGGAG | AAACTCCTCATCTATCGTGCATCCAACCT |
| light chain | ATATTAACTACAATGGGAAGTTCAAGGG | AGAATCTGGGATCCCTGCCAGGTTCAGTG |
| and IgG2a | CAAGGCCACACTGACTGCAGACAAATCC | GCAGTGGGTCTAGGACAGACTTCACCCTC |
| with a | TCCAGCACAGCCTACATGCAACTCAGCA | ACCATTAATCCTGTGGAGGCTGATGATGT |
| kappa light | GCCTGACATCTGACGACTCTGCGGTCTAC | TGCAACCTATTACTGTCAGCAAAGTAATG |
| chain) | TTCTGTACAAAGCGGGGACGCTATGCTTT | AGGATCCGTACACGTTCGGAGGGGGGAC |
| | GGACTACTGGGGTCAAGGAACCTCAGTC | CAAGCTGGAAATGAAA |
| | ACCGTCTCCTCA | (SEQ ID NO: 55) |
| | (SEQ ID NO: 54) | |
| ACI-8037- | CAAGTTACTCTAAAAGAGTCTGGCCCTGG | GATGTTTTGATGACCCAAACTCCACTCTC |
| 103H5- | GATATTGAAGCCCTCACAGACCCTCAGTC | CCTGCCTGTCAGTCTTGGGGATCAAGCCT |
| Ab2 (a | TGACTTGTTCTTTCTCTGGGTTTTCACTGA | CCATCTCTTGCAGATCTAGTCGGAGCATT |
| murine | GCACTTCTGGTATGGGTGTTGGCTGGATT | GTTCATAGTAATGGAAACACCTATTTAGA |
| IgG2a | CGTCAGCCTTCCGGGAAGGGTCTGGAGT | ATGGTACCTGCAGAAACCAGGCCAGTCT |
| isotype | GGCTGGCACACATTTGGTGGGATGATGA | CCAAAGCTCCTGATCTTCAAAGTTTCCAA |
| with a | TAAGTACTATAACCCATCCCTGAAGAGCC | CCGATTTTCTGGGGTCCCAGACAGGTTCA |
| kappa light | GGCTCACAATCTCCAAGGATTCCTCCAGA | GTGGCAGTGGATCAGGGACAGATTTCAC |
| chain) | AACCAGGTATTCCTCAAGATCACCAGTGT | ACTCAAGATCAGCAGAGTGGAGGCTGAG |
| | GGACACTGCAGATACTGCCACTTACTACT | GATCAGGGAGTTTATTACTGCTTTCAAGG |
| | GTGTTCGAAGGGGGACTAGGTTAGAGGA | TTCACATGTTCCGCTCACGTTCGGTGCTG |
| | CTACTTTGACTTCTGGGGCCAAGGCACCA | GGACCAAGCTGGAGCTGAAA |
| | CTCTCACAGTCTCCTCA | (SEQ ID NO: 69) |
| | (SEQ ID NO: 68) | |
| ACI-8037- | CAGGTTACTCTGAAAGAGTCTGGCCCTGG | GATGTTTTGATGACCCAAACTCCACTCTC |
| 109F4- | GATATTGCAGCCCTCCCAGACCCTCAGTC | CCTGCCTGTCAGTCTTGGAGATCAGGCCT |
| Ab1 (a | TGACTTGTTCTTTCTCTGGGTTTTCACTGA | CCATCTCTTGCAGATCTAGTCAGACCATT |
| murine | GCACTTCTGGTATGGGTGTGAGCTGGATT | GTACATAGTAATGGAAACACCTATTTGG |
| IgG2a | CGTCAGCCTTCAGGAAAGGGTCTGGAGT | AATGGTACCTGCAGAAACCAGGCCAGTC |
| isotype | GGCTGGCACACATTTACTGGGATGATGA | TCCAAACCTCCTGATCTACAAAGTTTCCA |
| with a | CAAGCGCTATAACCCATCCCTGAAGAGC | ACCGATTTTCTGGGGTCCCAGACAGGTTC |
| kappa light | CGGCTCACAATCTCCAAGGATACCTCCAG | AGTGGCAGTGGATCAGGGACAGATTTCA |
| chain) | AAACCAGGTTTTCCTCAAGATCACCAGTG | CACTCAAGATCAGCAGAGTGGAGGCTGA |
| | TGGACACTGCAGATGCTGGCACATACTA | GGATCTGGGAGTTTATTACTGCTTTCAAG |
| | CTGTGCTCGAAGACCTATTAGAACGGTA | GTTCACATGTTCCGCTCACGTTCGGTGCT |
| | GTAGATGCTATGGACTACTGGGGTCAAG | GGGACCAAGCTGGAGCTGAAA |
| | GAACCTCAGTCACCGTCTCCTTA | (SEQ ID NO: 79) |
| | (SEQ ID NO: 78) | |
| ACI-8039- | CAAGTTACTCTAAAAGAGTCTGGCCCTGG | GATGTTGTGATGACCCAAACTCCACTCTC |
| 306H5- | GATATTGAAGCCCTCACAGACCCTCAGTC | CCTGCCTGTCAGTCTTGGAGATCAAGCCT |
| Ab1 (a | TGACTTGTTCTTTCTCTGGGTTTTCACTGA | CCATCTCTTGCAGATCTAGTCAGAGCCTT |
| murine | GCACTTCTGGTATGGGTGTAGGCTGGATT | GTACACAGTAATGGAAACACCTATTTAC |
| IgG2a | CGTCAGCCTTCAGGGAAGGGTCTGGAGT | ATTGGTACCTGCAGAAGCCAGGCCAGTC |
| isotype | GGCTGGCACACATTTGGTGGGATGATGA | TCCAAACCTCCTGATCTACAAAGTTTCCA |
| with a | TAAGTACTATAACCCATCCCTGAAGAGTC | ACCGATTTTCTGGGGTCCCAGACAGGTTC |
| kappa light | AGCTCACAATCTCCAAGGATACCTCCAG | AGTGGCAGTGGATCAGGGACAGATTTCA |
| chain) | AAACCAGATATTCCTCAAGATCACCAGT | CACTCAAGATCAGCAGAGTGGAGGCTGA |
| | GTGGACACTGCAGATACTGCCACTTACTA | GGATCTGGGAGTTTATTTCTGCTCTCAAA |
| | CTGTGTTCGAAGCCTGTACTTTGACTACT | GTACACATGTTCCGCTCACGTTCGGTGCT |
| | GGGGCCAAGGCACCACTCTCACAGTCTC | GGGACCAAGCTGGAGCTGAAA |
| | CTCA | (SEQ ID NO: 89) |
| | (SEQ ID NO: 88) | |
| ACI-8039- | CAGGTCCAACTGCAGCAGCCTGGGGCTG | GACATTGTGATGACACAGTCTCCATCCTC |
| 307F7- | AGCTGGTGAGGCCTGGAGGTTCAGTGAA | CCTGGCTATGTCAGTAGGACAGAAGGTC |
| Ab2 (a | GCTGTCCTGCAAGGCTTCTGGCTACTCCT | ACTATGAGCTGCAAGTCCAGTCAGAGCC |
| murine | TCACCAACTACTGGATGAACTGGGTGAA | TTTTAAATAGTAGCAATCAAAAGAACTAT |
| IgG2a | GCAGAGGCCTGGACAAGGCCTTGAGTGG | TTGGCCTGGTACCAGCAGAAACCAGGAC |
| isotype | ATTGGCATGATTCATCCTTCCGATAGTGA | AGTCTCCTAAAATTCTGGTATACTTTGCA |
| with a | AACTAGGTTAAATCAGAAATTCAAGGAC | TCCACTAGGGAATCTGGGGTCCCTGATCG |
| kappa light | AAGGCCACATTGACTGTAGACAAATCAT | CTTCAGAGGCAGTGGATCTGGGACAGAT |
| chain) | CCAGCACAGCCTACTTGCAACTCAGCAG | TTCACTCTAACCATCAGCAGTGTGCAGGC |
| | CCCGACATCTGAGGACTCTGCGGTCTATT | TGAAGACCTGGCAGATTACTTCTGTCAGC |
| | ACTGTGCAAAATTACTACTATACTACTTT | AACATTATAGCCCTCCTCCGACGTTCGGT |
| | GACTACTGGGGCCAAGGCACCACTCTCA | GGAGGCACCAAGCTGGAAATCAAA |
| | CAGTCTCCTCA | (SEQ ID NO: 99) |
| | (SEQ ID NO: 98) | |
| ACI-8039- | CAGGTGCAACTGCAGCAGTCTGGGCCTC | GACATTTTGATGACCCAGTCTCCATCCTC |
| 309D9- | AGCTGGTTAGACCTGGGGCTTCAGTGAA | CCTGACTGTGTCAGCAGGAGAGAAGGTC |
| Ab2 (a | GATATCCTGCAAGGCTTCTGGTAACTCAT | ACTATGAGCTGCAAGTCCAGTCAGAGTCT |
| murine | TCACCAGCTACTGGATGCACTGGGTGAA | TTTAGCTAGTGGCAACCAAAATAACTACT |
| IgG2a | GCAGAGGCCTGGACAAGGTCTTGAGTGG | TGGCCTGGCACCAGCAGAAACCAGGACG |
| isotype | ATTGGCATGATTGATCCTTCCGATAGTGA | ATCTCCTAAAATACTGATAATTTGGGCAT |
| with a | GACTAGGTTAAATCAGAAGTTCAAGGAC | CCACTAGGGTATCTGGAGTCCCTGATCGC |
| kappa light | AAGGCCACATTGACTGTAGACAAATCCT | TTCATAGGCAGTGGGTCTGGGACGGATTT |
| chain) | CCAGCACAGCCTACATGCAACTCAGCAG | CACTCTGACCATCAACAGTGTGCAGGCTG |
| | CCCGACATCTGAGGACTCTGCGGTCTATT | AAGATCTGGCTGTTTATTACTGTCAGCAG |
| | ACTGTGCGGGGCTGTACTTTACTTTGGAC | TCCTACAGCGCTCCGCTCACGTTCGGTGC |
| | TACTGGGGTCAAGGAACCTCAGTCACCG | TGGGACCAAGCTGGAGCTGAAA |
| | TCTCCTCA | (SEQ ID NO: 109) |
| | (SEQ ID NO: 108) | |
| ACI-8041- | CAGGTGCAGCTGAAGGAGTCAGGACCTG | GATGTTGTGCTGACCCAGACTCCACTCAC |
| 1F11B5- | GCCTGGTGGCGCCCTCACAGAGCCTGTCC | TTTGTCGGTTACCATTGGACAACCAGCCT |
| Ab1 (a | ATCACATGCACTGTCTCAGGGTTCTCATT | CCATCTCTTGCAAGTCAAGTCAGAGCCTC |
| murine | AACCAACTATGGTGTAAGCTGGATTCGCC | TTAGATAGTGATGGAAGGACATATTTAAT |
| IgG2a | AGCCTCCAGGAAAGGGTCTGGAGTGGCT | TTGGTTGTTACAGAGGCCAGGCCAGTCTC |
| isotype | GGGAGTAATATGGGGTGACGGGAGCACA | CAAAGCGCCTAATCTATCTGGTGTCAACA |
| with a | AATTATCATTCAGCTCTCATATCCAGACT | CTGGACTCTGGAGTCCCTGACAGGTTCAC |
| kappa light | GAGCATCAGCAAGGATAACTCCAAGAGC | TGGCAGTGGATCAGGGACAGATTTCACA |
| chain) | CAAGTTTTCTTAAAACTGAACAGTCTGCA | CTGAAAATCAGCAGAGTGGAGGCTGAGG |
| | AACTGATGACACAGCCACGTACTACTGT | ATTTGGGAGTTTATTATTGCTGGCAAGGT |
| | ACCAAAGGAGCTGGCTCTTGGGGCCAAG | ACACATTTTCCGTGGACGTTCGGTGGAGG |
| | GGATTCCGGTCACTGTCTCTGCA | CACCAAGCTGGAAATCAAA |
| | (SEQ ID NO: 118) | (SEQ ID NO: 119) |
| ACI-8041- | CAGGTTCAGCTGCAACAGTCTGACGCTG | GACATCAAGATGACCCAGTCTCCATCTTC |
| 2B9H5- | AGTTGGTGAAACCTGGAGCTTCAGTGAA | CATGTATGCATCTCTAGGAGAGAGAGTC |
| Ab1 (a | GATATCCTGCAAGGTTTCTGGCTACACCT | ACTATCACTTGCAAGGCGAGTCAGGACA |
| murine | TCACTGACCATCCTATTCACTGGATGAAG | TTAATAGCTATTTAAGCTGGTTCCAGCAG |
| IgG2a | CAGAGGCCTGACCAGGGCCTGGAATGGA | AAACCAGGGAAATCTCCTAAGACCCTGA |
| isotype | TTGGATATTTTTATCCTAGAGATAGTAGT | TCTATCGTGCAAACAGATTGGTAGATGG |
| with a | ACTCACTACAATGAGAAGTTCAAGGGCA | GGTCCCATCAAGGTTCAGTGGCAGTGGA |
| kappa light | AGGCCACATTGACTGCAGACAAATCCTC | TCTGGGCAAGATTATTCTCTCACCATCAG |
| chain) | CAGCACAGCCTACATGCAGCTCAACAGC | CAGTCTGGACTATGAAGATATGGGAATTT |
| | CTGACATCTGAGGACTCTGCAGTCTACTT | ATTATTGTCTACAGTATGATGAGTTTCCT |
| | CTGTGCAAGATCGATGGGCTACTGGGGT | CCCACGTTCGGTGCTGGGACCAAGCTGG |
| | CAAGGAACCTCAGTCACCGTCTCCTCA | AGCTGAAA |
| | (SEQ ID NO: 128) | (SEQ ID NO: 129) |
| ACI-8041- | CAGGTCCAACTGCAGCAGTCTGGGGCTG | GATGTTTTGATGACCCAAACTCCACTCTC |
| 3G11B3- | AACTGGTGAAGCCTGGGGCTTCAGTGAA | CCTGCCTGTCAGTCTTGGAGATCAAGCCT |
| Ab1 (a | GTTGTCCTGCAAGGCTTCTGGCTACACCT | CCATCTCTTGCAGATCTAGTCAGAGCATT |
| murine | TCATCAACTACTATATATACTGGGTGAAA | GTACATAGTAATGGAATCACCTATTTAGA |
| IgG2a | CAGAGGCCTGGACAAGGCCTTGAGTGGA | ATGGTACCTGCAGAAACCAGGCCAGTCT |
| isotype | TTGGAGAGATTATTCCTAGCAATGATGGT | CCAAAGCTCCTAATCTACAAAGTTTCCAA |
| with a | ACTAACTTCAATGAGAAGTTCAAGACCA | CCGATTTTCTGGGGTCCCAGACAGGTTCA |
| kappa light | AGGCCACACTGACTGTAGACAAATCCTC | GTGGCAGTGGATCAGGGACAGATTTCAC |
| chain) | CAGCACAGCATACATGCAACTCAGCAGC | ACTCAAGATCAGCAGAGTGGAGGCTGAG |
| | CTGACATCTGAGGACTCTGCGGTCTATTA | GATCTGGGAGTTTATTACTGCTTTCAAGG |
| | CTGTACCGCGACAGGGACGGTCTGGGGC | TTCACATGTTCCGTGGACGTTCGGTGGAG |
| | CAAGGGACTCTGGTCACTGTCTCTGCA | GCACCAAGCTGGAAATCAAA |
| | (SEQ ID NO: 138) | (SEQ ID NO: 139) |
| ACI-8041- | CAGGTCCAACTGCAGCAGTCTGGGGCTG | GATGTTTTGATGACCCAAACTCCACTCTC |
| 4G6D2- | AACTGGTGAGGCCTGGGGCTTCAGTGAA | CCTGCCTGTCAGTCTTGGAGATCAAGCCT |
| Ab1 (a | GTTGTCCTGCAAGGCTTCTGGCTACACCT | CCATCTCTTGCAGATCTAGTCAGAGCATT |
| murine | TCACCAGCTATTATATGTACTGGGTGAAG | GTACATAGTAATGGAAACACCTATTTAG |
| IgG2a | CAGAGGCCTGGACAAGGCCTTGAGTGGA | AATGGTACCTGCAGAAACCAGGCCAGTC |
| isotype | TTGGAGAGATTATTCCTAGTAATGGTGAT | TCCAAAGCTCCTGATCTACAAAGTTTCCA |
| with a | ACTAACTTCAATGAGAAGTTCAGGAGCA | ACCGATTTTCTGGGGTCCCAGACAGGTTC |
| kappa light | AGGCCACACTGACTGTAGACAAATCCTC | AGTGGCAGTGGATCAGGGACAGATTTCA |
| chain) | CAGCACAGCATATATGCAACTCAGCAGC | CACTCAAGATCAGCAGAGTGGAGGCTGA |
| | CTGACATCTGAGGACTCTGCGGTCTATTA | GGATCTGGGAGTTTATTACTGCTTTCAAG |
| | CTGTACCGCGACAGGGACGGTCTGGGGC | GTTCACATGTTCCGTGGACGTTCGGTGGA |
| | CAAGGGACTCTGGTCACTGTCTCTGCA | GGCACCAAGCTGGAAATCAAA |
| | (SEQ ID NO: 148) | (SEQ ID NO: 149) |
| ACI-8041- | GAGGTCCAGCTGCAACAGTCTGGACCTG | GAAATTGTGCTCACCCAGTCTCCAACCAC |
| 5A2D4- | AGCTGGTGAAGCCTGGGGCTTCAGTGAA | CATGGCTGCATCTCCCGGGGAGAAGATC |
| Ab1 (a | GATATCCTGCAAGACTTCTGGATACACAT | ACTATCACCTGCAGTGGTAGCTCAAGTAT |
| murine | TCACTGAAAACACCATGCACTGGGTGAA | AATTTCCAATTACTTGCATTGGTATCAGC |
| IgG2a | GCAGATCCATGGAAAGAGCCTTGAGTGG | AGAAGCCAGGATTCTCCCCTAAACTCTTG |
| isotype | ATTGGAGGTATTAATCCTAACAATGGTGT | ATTTATAGGACATCCAATCTGGCTTCTGG |
| with a | TACTAACAACAACCAGAAGTTCGAGGGC | AGTCCCAACTCGCTTCAGTGGCAGTGGGT |
| kappa light | AAGGCCACTTTGACTGTAGACAAGTCCTC | CTGGGACCTCTTACTCTCTCACAATTGGC |
| chain) | CAGCACAGCCTACATGGAGCTCCGCAGC | ACCATGGAGGCTGAAGATGTTGCCACTT |
| | CTGACATCTGAGGATTCTGCAGTCTATTA | ACTACTGCCAGCAGGGTAGTAGTATACC |
| | CTGTGCAAGATCTGATTACGACGCCCTTT | ATTCACGTTCGGCTCGGGGACAAAGTTG |
| | ACTATTCTATGGACTGCTGGGGTCAAGGA | GAAATAAAA |
| | ACCTCAGTCACCGTCTCCTCA | (SEQ ID NO: 159) |
| | (SEQ ID NO: 158) | |
| ACI-8041- | GATGTGCAGCTTCAGGAGTCGGGACCTG | GATATTGTGATGACGCAGGCTGCATTCTC |
| 5A6H9- | GCCTGGTGAAACCTTCTCAGTCTCTGTCC | CAATCCAGTCACTCTTGGAACATCAGTTT |
| Ab1 (a | CTCACCTGCACTGTCACTGGCTACTCAAT | CCATCTCCTGCAGGTCTAGTAAGAGTCTC |
| murine | CACCAGTGATTATGCCTGGAACTGGATCC | CTACATAGTAATGGCATCACTTATTTGTA |
| IgG2a | GGCAGTTTCCAGGAAACAAACTGGAGTG | TTGGTATCTGCAGAAGCCAGGCCAGTCTC |
| isotype | GATGGGCTACATAGACTACAGTGGTATC | CTCAGCTCCTGATTTATCAGATGTCCAAC |
| with a | ACTAGCTACAACCCATCTCTCAAAAGTCG | CTTGCCTCAGGAGTCCCAGACAGGTTCAG |
| kappa light | AATCTCTATCACTCGAGACACATCCAAGA | TAGCAGTGGGTCAGGAACTGATTTCACA |
| chain) | ACCAGTTCTTCCTGCAGTTGAATTCTGTG | CTGAGAATCAGCAGAGTGGAGGCTGAGG |
| | ACTACTGAGGACTCAGCCACATATTACTG | ATGTGGGTGTTTATTACTGTGCTCAAAAT |
| | TGCAAGAATCTCTGGACTGGATTACGAC | CTAGAACGTCCGCTCACGTTCGGTGCTGG |
| | GTGAGGGCTGGCTGGTTTGCTTCCTGGGG | GACCAAGCTGGAGCTGAAA |
| | CCAAGGGACTCTGGTCACTGTCTCTGCA | (SEQ ID NO: 169) |
| | (SEQ ID NO: 168) | |
| ACI-8041- | GAGGTCCAGCTGCAACAGTCTGGACCTG | GAAATTGTGCTCACCCAGTCTCCAACCAC |
| 6C3C4- | AGCTGGTGAAGCCTGGGGCTTCAGTGAA | CATGGCTGCATCTCCCGGGGAGAAGATC |
| Ab1 (a | CATATCCTGCAAGACTTCTGGATACACAT | ACTATCACCTGCAGTGCCAGCTCAAGTAT |
| murine | TCACTGAATACACCATGCACTGGATGAA | AAGTTCCAATTACTTGCATTGGTATCAGC |
| IgG2a | GCAGAGCCATGGAAAGAGCCTTGAGTGG | AGAAGCCAGGATTCTCCCCTAAACTCTTG |
| isotype | ATTGGAGGTATTAATCCTAACAATGGTGT | ATTTATAGGACATCCAATCTGGCTTCTGG |
| with a | TACTGGCTACAACCAGAAGTTCAAGGGC | AGTCCCAGCTCGCTTCAGTGGCAGTGGGT |
| kappa light | AGGGCCACATTGACTGTAGACAAGTCCT | CTGGGACCTCTTACTCTCTCACAATTGGC |
| chain) | CCAGCACAGCCTACATGGAGCTCCGCAG | ACCATGGAGGCTGAAGATGTTGCCACTT |
| | CCTGACATCTGAGGATTCTGCAGTCTATT | ACTACTGCCAGCAGGGTAGTAGTATACC |
| | ACTGTGCAAGATCTGATTACGACGACCTT | ATTCACGTTCGGCTCGGGGACAAAGTTG |
| | TACTATGCTATGGACTACTGGGGTCAAGG | GAAATAAAA |
| | AACCTCAGTCACCGTCTCCTCA | (SEQ ID NO: 179) |
| | (SEQ ID NO: 178) | |
| ACI-8041- | GAAGTGATGCTGGTGGAGTCTGGGGGAG | GACATTGTGATGACACAGTCTCCATCCTC |
| 9D7E1- | GCTTAGTGAAGCCTGGAGGGTCCCTGAA | CCTGGCTATGTCAGTAGGACAGAAGGTC |
| Ab1 (a | ACTCTCCTGTGCAGCCTCTGGATTCACTT | ACTATGAGCTGCAAGTCCAGTCAGAGCC |
| murine | TCAGTAGCTATGCCATGTCTTGGGTTCGC | TTTTAAATAGTAGCAATCAAAAGAACTAT |
| IgG2a | CAGACTCCGGAGAAGAGGCTGGAGTGGG | TTGGCCTGGTACCAGCAGAAACCAGGAC |
| isotype | TCGCAACCATTAGTAGTGGTGGTAATTAC | AGTCTCCTAAACTTCTGGTATACTTTGCA |
| with a | ACCTACTGTCCCGACAGTGTGAAGGGGC | TCCACTAGGGAATCTGGGGTCCCTGATCG |
| kappa light | GATTCACCATCTCCAGAGACAATGCCAA | CTTCATAGGCAGTGGATCTGGGACAGATT |
| chain) | GAACACCCTGTACCTGCAAATGAGCAGT | TCACTCTTACCATCAGCAGTGTGCAGGCT |
| | CTGAGGTCTGAGGACACGGCCATGTATT | GAAGACCTGGCAGATTACTTCTGTCAGCA |
| | ACTGTGCAGCCTATGCTTACGACGACGG | ACATTATAGCACTCCTCTCACGTTCGGTG |
| | GTACTACTTTGACTACTGGGGCCAAGGCA | CTGGGACCAAGCTGGAGCTGAAA |
| | CCACTCTCACAGTCTCCTCA | (SEQ ID NO: 189) |
| | (SEQ ID NO: 188) | |
| ACI-8041- | GAAGTGATGCTGGTGGAGTCTGGGGGAG | GACATCCAGATGACACAGTCTCCATCCTC |
| 10H3B11- | GCTTAGTGAAGCCTGGAGGGTCCCTGAA | ACTGTCTGCATCTCTGGGAGGCAAAGTCA |
| Ab1 (a | ACTCTCCTGTGCAGCCTCTGGATTCACTT | CCATCACTTGCAAGGCAAGCCAAGACAT |
| murine | TCAGTAGCTATGCCATGTCTTGGGTTCGC | TAACAAGTATATAGCTTGGTACCAACAC |
| IgG2a | CAGACTCCGGAGAAGAGGCTGGAGTGGG | AAGCCTGGAAAAGGTCCTAGGCTGCTCA |
| isotype | TCGCAACCATTAGTAGTGGTGGTAATTAC | TTCATTACACATCTACCTTACAGCCAGGC |
| with a | ACCTACTGTCCCGACAGTGTGAAGGGGC | ATCCCATCAAGGTTCAGTGGAAGTGGGT |
| kappa light | GATTCACCATCTCCAGAGACAATGCCAA | CTGGGAGAGATTATTCCTTCAGCATCAGC |
| chain) | GAACACCCTGTACCTGCAAATGAGCAGT | AACCTGGGGCCTGAAGATATTGCAACTT |
| | CTGAGGTCTGAGGACACGGCCATGTATT | ATTATTGTCTACAGTATGATAATCTTCTG |
| | ACTGTGCAGCCTATGCTTACGACGACGG | ACGTTCGGTGGAGGCACCAAGCTGGAAA |
| | GTACTACTTTGACTACTGGGGCCAAGGCA | TCAAA |
| | CCACTCTCACAGTCTCCTCA | (SEQ ID NO: 199) |
| | (SEQ ID NO: 188) | |

TABLE 3
Amino acid sequences of the Heavy Chain and Light Chain Variable
domains (VH and VL)

| Antibody | VH | VH CDR1 | VH CDR2 | VH CDR3 |
|---|---|---|---|---|
| ACI-24- | QVTLKESGPGILQSSQTLS | TSGMGVS | HIYWDDDKRYNP | RRNGYDGGFAY |
| 41F12- | LTCSFSGFSLSTSGMGVS | (SEQ ID NO: | SLKS | (SEQ ID NO: |
| Ab2 and | WIRQPSGKGLEWLAHIYW | 11) | (SEQ ID NO: | 13) |
| ACI-24- | DDDKRYNPSLKSRLTISK | | 12) | |
| 41F12- | DTSRNQVFLKITSVDTAD | | | |
| Ab3 (a | TATYYCARRRNGYDGGF | | | |
| murine | AYWGQGTLVTVSA | | | |
| IgG2a | (SEQ ID NO: 10) | | | |
| isotype) | VL | VL CDR1 | VL CDR2 | VL CDR3 |
| and | DVLMTQTPLSLPVSLGDQ | RSSQSIVHSNGNT | KVSNRFS | FQGSHVPLT |
| ACI-24- | ASISCRSSQSIVHSNGNTY | YLE | (SEQ ID NO: | (SEQ ID NO: |
| 41F12- | LEWYLQKPGQSPKLLIYK | (SEQ ID NO: | 16) | 17) |
| Ab3 (a | VSNRFSGVPDRFSGSGSGT | 15) | | |
| human | DFTLKISRVEAEDLGVYY | | | |
| IgG1 | CFQGSHVPLTFGAGTKLE | | | |
| isotype) | LK | | | |
| | (SEQ ID NO: 14) | | | |
| Antibody | VH | VH CDR1 | VH CDR2 | VH CDR3 |
| ACI-31- | EVQLVESGGDLVKPGGSL | TYGMS | TISSGRSYTYYAD | NSNNQRDYYAM |
| 25B1-Ab2 | KLSCAASRFTFSTYGMSW | (SEQ ID NO: | SVKG | DY |
| (two | VRQTPDKRLEWVATISSG | 31) | (SEQ ID NO: | (SEQ ID NO: |
| isotypes: a | RSYTYYADSVKGRFTISR | | 32) | 33) |
| murine | DNAKNTLYLQMSSLKSED | | | |
| IgG2b with | TAMYYCARNSNNQRDYY | | | |
| a lambda | AMDYWGQGTSVTVSS | | | |
| light chain | (SEQ ID NO: 30) | | | |
| and a | VL | VL CDR1 | VL CDR2 | VL CDR3 |
| murine | QLVLTQSSSASFSLGASAK | TLSSQHITYTIE | GSHSTGD | GVGDAIKEQFVY |
| IgG2a with | LTCTLSSQHITYTIEWYQQ | (SEQ ID NO: | (SEQ ID NO: | V |
| a lambda | QPLKPPKYVMVLEKDGSH | 35) | 36) | (SEQ ID NO: |
| light chain) | STGDGIPDRFSGSSSGADR | | | 37) |
| | YLSISNIQPEDEAVYICGV | | | |
| | GDAIKEQFVYVFGGGTKV | | | |
| | TVL | | | |
| | (SEQ ID NO: 34) | | | |
| Antibody | VH | VH CDR1 | VH CDR2 | VH CDR3 |
| ACI-31- | QVQLQQSGPELVKPGASV | SSWLN | RIYPGDGDINYNG | RGRYALDY |
| 30C11- | KISCKASGYAFSSSWLNW | (SEQ ID NO: | KFKG | (SEQ ID NO: |
| Ab1 (two | VKQRPGEGLEWIGRIYPG | 41) | (SEQ ID NO: | 43) |
| isotypes: a | DGDINYNGKFKGKATLTA | | 42) | |
| murine | DKSSSTAYMQLSSLTSDD | | | |
| IgG2b with | SAVYFCTKRGRYALDYW | | | |
| a kappa | GQGTSVTVSS | | | |
| light chain | (SEQ ID NO: 40) | | | |
| and a | VL | VL CDR1 | VL CDR2 | VL CDR3 |
| murine | DIVLTQSPASLAVSLGQRA | RASKSVDNYGKS | RASNLES | QQSNEDPYT |
| IgG2a with | TISCRASKSVDNYGKSFM | FMH | (SEQ ID NO: | (SEQ ID NO: |
| a kappa | HWFQQKPGQPPKLLIYRA | (SEQ ID NO: | 46) | 47) |
| light chain) | SNLESGIPARFSGSGSRTD | 45) | | |
| | FTLTINPVEADDVATYYC | | | |
| | QQSNEDPYTFGGGTKLEM | | | |
| | K | | | |
| | (SEQ ID NO: 44) | | | |
| Antibody | VH | VH CDR1 | VH CDR2 | VH CDR3 |
| ACI-8037- | QVTLKESGPGILKPSQTLS | TSGMGVG | HIWWDDDKYYN | RGTRLEDYFDF |
| 103H5-Ab2 | LTCSFSGFSLSTSGMGVG | (SEQ ID NO: | PSLKS | (SEQ ID NO: |
| (a murine | WIRQPSGKGLEWLAHIW | 61) | (SEQ ID NO: | 63) |
| IgG2a | WDDDKYYNPSLKSRLTIS | | 62) | |
| isotype | KDSSRNQVFLKITSVDTA | | | |
| with a | DTATYYCVRRGTRLEDYF | | | |
| kappa light | DFWGQGTTLTVSS | | | |
| chain) | (SEQ ID NO: 60) | | | |
| | VL | VL CDR1 | VL CDR2 | VL CDR3 |
| | DVLMTQTPLSLPVSLGDQ | RSSRSIVHSNGNT | KVSNRFS | FQGSHVPLT |
| | ASISCRSSRSIVHSNGNTY | YLE | (SEQ ID NO: | (SEQ ID NO: |
| | LEWYLQKPGQSPKLLIFK | (SEQ ID NO: | 16) | 17) |
| | VSNRFSGVPDRFSGSGSGT | 65) | | |
| | DFTLKISRVEAEDQGVYY | | | |
| | CFQGSHVPLTFGAGTKLE | | | |
| | LK | | | |
| | (SEQ ID NO: 64) | | | |
| Antibody | VH | VH CDR1 | VH CDR2 | VH CDR3 |
| ACI-8037- | QVTLKESGPGILQPSQTLS | TSGMGVS | HIYWDDDKRYNP | RPIRTVVDAMDY |
| 109F4-Ab1 | LTCSFSGFSLSTSGMGVS | (SEQ ID NO: | SLKS | (SEQ ID NO: |
| (a murine | WIRQPSGKGLEWLAHIYW | 11) | (SEQ ID NO: | 73) |
| IgG2a | DDDKRYNPSLKSRLTISK | | 12) | |
| isotype | DTSRNQVFLKITSVDTAD | | | |
| with a | AGTYYCARRPIRTVVDAM | | | |
| kappa light | DYWGQGTSVTVSL | | | |
| chain) | (SEQ ID NO: 70) | | | |
| | VL | VL CDR1 | VL CDR2 | VL CDR3 |
| | DVLMTQTPLSLPVSLGDQ | RSSQTIVHSNGNT | KVSNRFS | FQGSHVPLT |
| | ASISCRSSQTIVHSNGNTY | YLE | (SEQ ID NO: | (SEQ ID NO: |
| | LEWYLQKPGQSPNLLIYK | (SEQ ID NO: | 16) | 17) |
| | VSNRFSGVPDRFSGSGSGT | 75) | | |
| | DFTLKISRVEAEDLGVYY | | | |
| | CFQGSHVPLTFGAGTKLE | | | |
| | LK | | | |
| | (SEQ ID NO: 74) | | | |
| Antibody | VH | VH CDR1 | VH CDR2 | VH CDR3 |
| ACI-8039- | QVTLKESGPGILKPSQTLS | TSGMGVG | HIWWDDDKYYN | SLYFDY |
| 306H5-Ab1 | LTCSFSGFSLSTSGMGVG | (SEQ ID NO: | PSLKS | (SEQ ID NO: |
| (a murine | WIRQPSGKGLEWLAHIW | 61) | (SEQ ID NO: | 83) |
| IgG2a | WDDDKYYNPSLKSQLTIS | | 62) | |
| isotype | KDTSRNQIFLKITSVDTAD | | | |
| with a | TATYYCVRSLYFDYWGQ | | | |
| kappa light | GTTLTVSS | | | |
| chain) | (SEQ ID NO: 80) | | | |
| | VL | VL CDR1 | VL CDR2 | VL CDR3 |
| | DVVMTQTPLSLPVSLGDQ | RSSQSLVHSNGN | KVSNRFS | SQSTHVPLT |
| | ASISCRSSQSLVHSNGNTY | TYLH | (SEQ ID NO: | (SEQ ID NO: |
| | LHWYLQKPGQSPKLLIYK | (SEQ ID NO: | 16) | 87) |
| | VSNRFSGVPDRFSGSGSGT | 85) | | |
| | DFTLKISRVEAEDLGVYFC | | | |
| | SQSTHVPLTFGAGTKLEL | | | |
| | K | | | |
| | (SEQ ID NO: 84) | | | |
| Antibody | VH | VH CDR1 | VH CDR2 | VH CDR3 |
| ACI-8039- | QVQLQQPGAELVRPGGSV | NYWMN | MIHPSDSETRLNQ | LLLYYFDY |
| 307F7-Ab2 | KLSCKASGYSFTNYWMN | (SEQ ID NO: | KFKD | (SEQ ID NO: |
| (a murine | WVKQRPGQGLEWIGMIH | 91) | (SEQ ID NO: | 93) |
| IgG2a | PSDSETRLNQKFKDKATL | | 92) | |
| isotype | TVDKSSSTAYLQLSSPTSE | | | |
| with a | DSAVYYCAKLLLYYFDY | | | |
| kappa light | WGQGTTLTVSS | | | |
| chain) | (SEQ ID NO: 90) | | | |
| | VL | VL CDR1 | VL CDR2 | VL CDR3 |
| | DIVMTQSPSSLAMSVGQK | KSSQSLLNSSNQK | FASTRES | QQHYSPPPT |
| | VTMSCKSSQSLLNSSNQK | NYLA | (SEQ ID NO: | (SEQ ID NO: |
| | NYLAWYQQKPGQSPKILV | (SEQ ID NO: | 96) | 97) |
| | YFASTRESGVPDRFRGSGS | 95) | | |
| | GTDFTLTISSVQAEDLADY | | | |
| | FCQQHYSPPPTFGGGTKL | | | |
| | EIK | | | |
| | (SEQ ID NO: 94) | | | |
| Antibody | VH | VH CDR1 | VH CDR2 | VH CDR3 |
| ACI-8039- | QVQLQQSGPQLVRPGASV | SYWMH | MIDPSDSETRLNQ | LYFTLDY |
| 309D9-Ab2 | KISCKASGNSFTSYWMH | (SEQ ID NO: | KFKD | (SEQ ID NO: |
| (a murine | WVKQRPGQGLEWIGMID | 101) | (SEQ ID NO: | 103) |
| IgG2a | PSDSETRLNQKFKDKATL | | 102) | |
| isotype | TVDKSSSTAYMQLSSPTSE | | | |
| with a | DSAVYYCAGLYFTLDYW | | | |
| kappa light | GQGTSVTVSS | | | |
| chain) | (SEQ ID NO: 100) | | | |
| | VL | VL CDR1 | VL CDR2 | VL CDR3 |
| | DILMTQSPSSLTVSAGEKV | KSSQSLLASGNQ | WASTRVS | QQSYSAPLT |
| | TMSCKSSQSLLASGNQNN | NNYLA | (SEQ ID NO: | (SEQ ID NO: |
| | YLAWHQQKPGRSPKILII | (SEQ ID NO: | 106) | 107) |
| | WASTRVSGVPDRFIGSGS | 105) | | |
| | GTDFTLTINSVQAEDLAV | | | |
| | YYCQQSYSAPLTFGAGTK | | | |
| | LELK | | | |
| | (SEQ ID NO: 104) | | | |
| Antibody | VH | VH CDR1 | VH CDR2 | VH CDR3 |
| ACI-8041- | QVQLKESGPGLVAPSQSL | NYGVS | VIWGDGSTNYHS | GAGS |
| 1F11B5-Ab1 | SITCTVSGFSLTNYGVSWI | (SEQ ID NO: | ALIS | (SEQ ID NO: |
| (a murine | RQPPGKGLEWLGVIWGD | 111) | (SEQ ID NO: | 113) |
| IgG2a | GSTNYHSALISRLSISKDN | | 112) | |
| isotype | SKSQVFLKLNSLQTDDTA | | | |
| with a | TYYCTKGAGSWGQGIPVT | | | |
| kappa light | VSA | | | |
| chain) | (SEQ ID NO: 110) | | | |
| | VL | VL CDR1 | VL CDR2 | VL CDR3 |
| | DVVLTQTPLTLSVTIGQPA | KSSQSLLDSDGRT | LVSTLDS | WQGTHFPWT |
| | SISCKSSQSLLDSDGRTYLI | YLI | (SEQ ID NO: | (SEQ ID NO: |
| | WLLQRPGQSPKRLIYLVS | (SEQ ID NO: | 116) | 117) |
| | TLDSGVPDRFTGSGSGTD | 115) | | |
| | FTLKISRVEAEDLGVYYC | | | |
| | WQGTHFPWTFGGGTKLEI | | | |
| | K | | | |
| | (SEQ ID NO: 114) | | | |
| Antibody | VH | VH CDR1 | VH CDR2 | VH CDR3 |
| ACI-8041- | QVQLQQSDAELVKPGASV | DHPIH | YFYPRDSSTHYN | SMGY |
| 2B9H5-Ab1 | KISCKVSGYTFTDHPIHW | (SEQ ID NO: | EKFKG | (SEQ ID NO: |
| (a murine | MKQRPDQGLEWIGYFYPR | 121) | (SEQ ID NO: | 123) |
| IgG2a | DSSTHYNEKFKGKATLTA | | 122) | |
| isotype | DKSSSTAYMQLNSLTSED | | | |
| with a | SAVYFCARSMGYWGQGT | | | |
| kappa light | SVTVSS | | | |
| chain) | (SEQ ID NO: 120) | | | |
| | VL | VL CDR1 | VL CDR2 | VL CDR3 |
| | DIKMTQSPSSMYASLGER | KASQDINSYLS | RANRLVD | LQYDEFPPT |
| | VTITCKASQDINSYLSWFQ | (SEQ ID NO: | (SEQ ID NO: | (SEQ ID NO: |
| | QKPGKSPKTLIYRANRLV | 125) | 126) | 127) |
| | DGVPSRFSGSGSGQDYSL | | | |
| | TISSLDYEDMGIYYCLQY | | | |
| | DEFPPTFGAGTKLELK | | | |
| | (SEQ ID NO: 124) | | | |
| Antibody | VH | VH CDR1 | VH CDR2 | VH CDR3 |
| ACI-8041- | QVQLQQSGAELVKPGASV | NYYIY | EIIPSNDGTNFNE | TGTV |
| 3G11B3-Ab1 | KLSCKASGYTFINYYIYW | (SEQ ID NO: | KFKT | (SEQ ID NO: |
| (a murine | VKQRPGQGLEWIGEIIPSN | 131) | (SEQ ID NO: | 133) |
| IgG2a | DGTNFNEKFKTKATLTVD | | 132) | |
| isotype | KSSSTAYMQLSSLTSEDSA | | | |
| with a | VYYCTATGTVWGQGTLV | | | |
| kappa light | TVSA | | | |
| chain) | (SEQ ID NO: 130) | | | |
| | VL | VL CDR1 | VL CDR2 | VL CDR3 |
| | DVLMTQTPLSLPVSLGDQ | RSSQSIVHSNGIT | KVSNRFS | FQGSHVPWT |
| | ASISCRSSQSIVHSNGITYL | YLE | (SEQ ID NO: | (SEQ ID NO: |
| | EWYLQKPGQSPKLLIYKV | (SEQ ID NO: | 16) | 137) |
| | SNRFSGVPDRFSGSGSGTD | 135) | | |
| | FTLKISRVEAEDLGVYYCF | | | |
| | QGSHVPWTFGGGTKLEIK | | | |
| | (SEQ ID NO: 134) | | | |
| Antibody | VH | VH CDR1 | VH CDR2 | VH CDR3 |
| ACI-8041- | QVQLQQSGAELVRPGASV | SYYMY | EIIPSNGDTNFNE | TGTV |
| 4G6D2-Ab1 | KLSCKASGYTFTSYYMY | (SEQ ID NO: | KFRS | (SEQ ID NO: |
| (a murine | WVKQRPGQGLEWIGEIIPS | 141) | (SEQ ID NO: | 133) |
| IgG2a | NGDTNFNEKFRSKATLTV | | 142) | |
| isotype | DKSSSTAYMQLSSLTSEDS | | | |
| with a | AVYYCTATGTVWGQGTL | | | |
| kappa light | VTVSA | | | |
| chain) | (SEQ ID NO: 140) | | | |
| | VL | VL CDR1 | VL CDR2 | VL CDR3 |
| | DVLMTQTPLSLPVSLGDQ | RSSQSIVHSNGNT | KVSNRFS | FQGSHVPWT |
| | ASISCRSSQSIVHSNGNTY | YLE | (SEQ ID NO: | (SEQ ID NO: |
| | LEWYLQKPGQSPKLLIYK | (SEQ ID NO: | 16) | 137) |
| | VSNRFSGVPDRFSGSGSGT | 15) | | |
| | DFTLKISRVEAEDLGVYY | | | |
| | CFQGSHVPWTFGGGTKLE | | | |
| | IK | | | |
| | (SEQ ID NO: 144) | | | |
| Antibody | VH | VH CDR1 | VH CDR2 | VH CDR3 |
| ACI-8041- | EVQLQQSGPELVKPGASV | ENTMH | GINPNNGVTNNN | SDYDALYYSMDC |
| 5A2D4-Ab1 | KISCKTSGYTFTENTMHW | (SEQ ID NO: | QKFEG | (SEQ ID NO: |
| (a murine | VKQIHGKSLEWIGGINPN | 151) | (SEQ ID NO: | 153) |
| IgG2a | NGVTNNNQKFEGKATLT | | 152) | |
| isotype | VDKSSSTAYMELRSLTSE | | | |
| with a | DSAVYYCARSDYDALYY | | | |
| kappa light | SMDCWGQGTSVTVSS | | | |
| chain) | (SEQ ID NO: 150) | | | |
| | VL | VL CDR1 | VL CDR2 | VL CDR3 |
| | EIVLTQSPTTMAASPGEKI | SGSSSIISNYLH | RTSNLAS | QQGSSIPFT |
| | TITCSGSSSIISNYLHWYQ | (SEQ ID NO: | (SEQ ID NO: | (SEQ ID NO: |
| | QKPGFSPKLLIYRTSNLAS | 155) | 156) | 157) |
| | GVPTRFSGSGSGTSYSLTI | | | |
| | GTMEAEDVATYYCQQGS | | | |
| | SIPFTFGSGTKLEIK | | | |
| | (SEQ ID NO: 154) | | | |
| Antibody | VH | VH CDR1 | VH CDR2 | VH CDR3 |
| ACI-8041- | DVQLQESGPGLVKPSQSL | SDYAWN | YIDYSGITSYNPS | ISGLDYDVRAGW |
| 5A6H9-Ab1 | SLTCTVTGYSITSDYAWN | (SEQ ID NO: | LKS | FAS |
| (a murine | WIRQFPGNKLEWMGYID | 161) | (SEQ ID NO: | (SEQ ID NO: |
| IgG2a | YSGITSYNPSLKSRISITRD | | 162) | 163) |
| isotype | TSKNQFFLQLNSVTTEDS | | | |
| with a | ATYYCARISGLDYDVRAG | | | |
| kappa light | WFASWGQGTLVTVSA | | | |
| chain) | (SEQ ID NO: 160) | | | |
| | VL | VL CDR1 | VL CDR2 | VL CDR3 |
| | DIVMTQAAFSNPVTLGTS | RSSKSLLHSNGIT | QMSNLAS | AQNLERPLT |
| | VSISCRSSKSLLHSNGITYL | YLY | (SEQ ID NO: | (SEQ ID NO: |
| | YWYLQKPGQSPQLLIYQM | (SEQ ID NO: | 166) | 167) |
| | SNLASGVPDRFSSSGSGTD | 165) | | |
| | FTLRISRVEAEDVGVYYC | | | |
| | AQNLERPLTFGAGTKLEL | | | |
| | K | | | |
| | (SEQ ID NO: 164) | | | |
| Antibody | VH | VH CDR1 | VH CDR2 | VH CDR3 |
| ACI-8041- | EVQLQQSGPELVKPGASV | EYTMH | GINPNNGVTGYN | SDYDDLYYAMD |
| 6C3C4-Ab1 | NISCKTSGYTFTEYTMHW | (SEQ ID NO: | QKFKG | Y |
| (a murine | MKQSHGKSLEWIGGINPN | 171) | (SEQ ID NO: | (SEQ ID NO: |
| IgG2a | NGVTGYNQKFKGRATLT | | 172) | 173) |
| isotype | VDKSSSTAYMELRSLTSE | | | |
| with a | DSAVYYCARSDYDDLYY | | | |
| kappa light | AMDYWGQGTSVTVSS | | | |
| chain) | (SEQ ID NO: 170) | | | |
| | VL | VL CDR1 | VL CDR2 | VL CDR3 |
| | EIVLTQSPTTMAASPGEKI | SASSSISSNYLH | RTSNLAS | QQGSSIPFT |
| | TITCSASSSISSNYLHWYQ | (SEQ ID NO: | (SEQ ID NO: | (SEQ ID NO: |
| | QKPGFSPKLLIYRTSNLAS | 175) | 156) | 157) |
| | GVPARFSGSGSGTSYSLTI | | | |
| | GTMEAEDVATYYCQQGS | | | |
| | SIPFTFGSGTKLEIK | | | |
| | (SEQ ID NO: 174) | | | |
| Antibody | VH | VH CDR1 | VH CDR2 | VH CDR3 |
| ACI-8041- | EVMLVESGGGLVKPGGSL | SYAMS | TISSGGNYTYCPD | YAYDDGYYFDY |
| 9D7E1-Ab1 | KLSCAASGFTFSSYAMSW | (SEQ ID NO: | SVKG | (SEQ ID NO: |
| (a murine | VRQTPEKRLEWVATISSG | 181) | (SEQ ID NO: | 183) |
| IgG2a | GNYTYCPDSVKGRFTISR | | 182) | |
| isotype | DNAKNTLYLQMSSLRSED | | | |
| with a | TAMYYCAAYAYDDGYYF | | | |
| kappa light | DYWGQGTTLTVSS | | | |
| chain) | (SEQ ID NO: 180) | | | |
| | VL | VL CDR1 | VL CDR2 | VL CDR3 |
| | DIVMTQSPSSLAMSVGQK | KSSQSLLNSSNQK | FASTRES | QQHYSTPLT |
| | VTMSCKSSQSLLNSSNQK | NYLA | (SEQ ID NO: | (SEQ ID NO: |
| | NYLAWYQQKPGQSPKLL | (SEQ ID NO: | 96) | 187) |
| | VYFASTRESGVPDRFIGSG | 95) | | |
| | SGTDFTLTISSVQAEDLAD | | | |
| | YFCQQHYSTPLTFGAGTK | | | |
| | LELK | | | |
| | (SEQ ID NO: 184) | | | |
| Antibody | VH | VH CDR1 | VH CDR2 | VH CDR3 |
| ACI-8041- | EVMLVESGGGLVKPGGSL | SYAMS | TISSGGNYTYCPD | YAYDDGYYFDY |
| 10H3B11-Ab1 | KLSCAASGFTFSSYAMSW | (SEQ ID NO: | SVKG | (SEQ ID NO: |
| (a murine | VRQTPEKRLEWVATISSG | 181) | (SEQ ID NO: | 183) |
| IgG2a | GNYTYCPDSVKGRFTISR | | 182) | |
| isotype | DNAKNTLYLQMSSLRSED | | | |
| with a | TAMYYCAAYAYDDGYYF | | | |
| kappa light | DYWGQGTTLTVSS | | | |
| chain) | (SEQ ID NO: 180) | | | |
| | VL | VL CDR1 | VL CDR2 | VL CDR3 |
| | DIQMTQSPSSLSASLGGKV | KASQDINKYIA | YTSTLQP | LQYDNLLT |
| | TITCKASQDINKYIAWYQ | (SEQ ID NO: | (SEQ ID NO: | (SEQ ID NO: |
| | HKPGKGPRLLIHYTSTLQP | 195) | 196) | 197) |
| | GIPSRFSGSGSGRDYSFSIS | | | |
| | NLGPEDIATYYCLQYDNL | | | |
| | LTFGGGTKLEIK | | | |
| | (SEQ ID NO: 194) | | | |

TABLE 4
Amino acid sequences of the Heavy Chain (HC) and Light Chain (LC)
variable and constant regions

| Antibody | HC | LC |
|---|---|---|
| ACI-24- | QVTLKESGPGILQSSQTLSLTCSFSGFSLSTS | DVLMTQTPLSLPVSLGDQASISCRSSQSIVH |
| 41F12- | GMGVSWIRQPSGKGLEWLAHIYWDDDKR | SNGNTYLEWYLQKPGQSPKLLIYKVSNRFS |
| Ab2 | YNPSLKSRLTISKDTSRNQVFLKITSVDTAD | GVPDRFSGSGSGTDFTLKISRVEAEDLGVY |
| | TATYYCARRRNGYDGGFAYWGQGTLVTV | YCFQGSHVPLTFGAGTKLELKRADAAPTVS |
| | SAAKTTPPSVYPLAPGCGDTTGSSVTLGCL | IFPPSSEQLTSGGASVVCFLNNFYPKDINVK |
| | VKGYFPESVTVTWNSGSLSSSVHTFPALLQ | WKIDGSERQNGVLNSWTDQDSKDSTYSMS |
| | SGLYTMSSSVTVPSSTWPSQTVTCSVAHPA | STLTLTKDEYERHNSYTCEATHKTSTSPIVK |
| | SSTTVDKKLEPSGPISTINPCPPCKECHKCPA | SFNRNEC |
| | PNLEGGPSVFIFPPNIKDVLMISLTPKVTCV | (SEQ ID NO: 21) |
| | VVDVSEDDPDVQISWFVNNVEVHTAQTQT | |
| | HREDYNSTIRVVSTLPIQHQDWMSGKEFKC | |
| | KVNNKDLPSPIERTISKIKGLVRAPQVYILPP | |
| | PAEQLSRKDVSLTCLVVGFNPGDISVEWTS | |
| | NGHTEENYKDTAPVLDSDGSYFIYSKLNM | |
| | KTSKWEKTDSFSCNVRHEGLKNYYLKKTIS | |
| | RSPGK | |
| | (SEQ ID NO: 20) | |
| ACI-24- | QVTLKESGPGILQSSQTLSLTCSFSGFSLSTS | DVLMTQTPLSLPVSLGDQASISCRSSQSIVH |
| 41F12- | GMGVSWIRQPSGKGLEWLAHIYWDDDKR | SNGNTYLEWYLQKPGQSPKLLIYKVSNRFS |
| Ab3 | YNPSLKSRLTISKDTSRNQVFLKITSVDTAD | GVPDRFSGSGSGTDFTLKISRVEAEDLGVY |
| (IgG2a | TATYYCARRRNGYDGGFAYWGQGTLVTV | YCFQGSHVPLTFGAGTKLELKRADAAPTVS |
| isotype) | SAAKTTAPSVYPLAPVCGDTTGSSVTLGCL | IFPPSSEQLTSGGASVVCFLNNFYPKDINVK |
| | VKGYFPEPVTLTWNSGSLSSGVHTFPAVLQ | WKIDGSERQNGVLNSWTDQDSKDSTYSMS |
| | SDLYTLSSSVTVTSSTWPSQSITCNVAHPAS | STLTLTKDEYERHNSYTCEATHKTSTSPIVK |
| | STKVDKKIEPRGPTIKPCPPCKCPAPNLLGG | SFNRNEC |
| | PSVFIFPPKIKDVLMISLSPIVTCVVVDVSED | (SEQ ID NO: 23) |
| | DPDVQISWFVNNVEVHTAQTQTHREDYNS | |
| | TLRVVSALPIQHQDWMSGKEFKCKVNNKD | |
| | LPAPIERTISKPKGSVRAPQVYVLPPPEEEM | |
| | TKKQVTLTCMVTDFMPEDIYVEWTNNGKT | |
| | ELNYKNTEPVLDSDGSYFMYSKLRVEKKN | |
| | WVERNSYSCSVVHEGLHNHHTTKSFSRTP | |
| | GK | |
| | (SEQ ID NO: 22) | |
| ACI-24- | QVTLKESGPGILQSSQTLSLTCSFSGFSLSTS | DVLMTQTPLSLPVSLGDQASISCRSSQSIVH |
| 41F12- | GMGVSWIRQPSGKGLEWLAHIYWDDDKR | SNGNTYLEWYLQKPGQSPKLLIYKVSNRFS |
| Ab3 | YNPSLKSRLTISKDTSRNQVFLKITSVDTAD | GVPDRFSGSGSGTDFTLKISRVEAEDLGVY |
| (hIgG1 | TATYYCARRRNGYDGGFAYWGQGTLVTV | YCFQGSHVPLTFGAGTKLELKRTVAAPSVF |
| isotype) | SAASTKGPSVFPLAPSSKSTSGGTAALGCL | IFPPSDEQLKSGTASVVCLLNNFYPREAKV |
| | VKDYFPEPVTVSWNSGALTSGVHTFPAVL | QWKVDNALQSGNSQESVTEQDSKDSTYSL |
| | QSSGLYSLSSVVTVPSSSLGTQTYICNVNHK | SSTLTLSKADYEKHKVYACEVTHQGLSSPV |
| | PSNTKVDKRVEPKSCDKTHTCPPCPAPELL | TKSFNRGEC |
| | GGPSVFLFPPKPKDTLMISRTPEVTCVVVD | (SEQ ID NO: 25) |
| | VSHEDPEVKFNWYVDGVEVHNAKTKPREE | |
| | QYNSTYRVVSVLTVLHQDWLNGKEYKCK | |
| | VSNKALPAPIEKTISKAKGQPREPQVYTLPP | |
| | SREEMTKNQVSLTCLVKGFYPSDIAVEWES | |
| | NGQPENNYKTTPPVLDSDGSFFLYSKLTVD | |
| | KSRWQQGNVFSCSVMHEALHNHYTQKSLS | |
| | LSPGK | |
| | (SEQ ID NO: 24) | |
| ACI-31- | EVQLVESGGDLVKPGGSLKLSCAASRFTFS | QLVLTQSSSASFSLGASAKLTCTLSSQHITY |
| 25B1-Ab2 | TYGMSWVRQTPDKRLEWVATISSGRSYTY | TIEWYQQQPLKPPKYVMVLEKDGSHSTGD |
| (murine | YADSVKGRFTISRDNAKNTLYLQMSSLKSE | GIPDRFSGSSSGADRYLSISNIQPEDEAVYIC |
| IgG2b with | DTAMYYCARNSNNQRDYYAMDYWGQGT | GVGDAIKEQFVYVFGGGTKVTVLGQPKSS |
| a lambda | SVTVSSAKTTPPSVYPLAPGCGDTTGSSVTL | PSVTLFPPSSEELETNKATLVCTITDFYPGV |
| light | GCLVKGYFPESVTVTWNSGSLSSSVHTFPA | VTVDWKVDGTPVTQGMETTQPSKQSNNK |
| chain) | LLQSGLYTMSSSVTVPSSTWPSQTVTCSVA | YMASSYLTLTARAWERHSSYSCQVTHEGH |
| | HPASSTTVDKKLEPSGPISTINPCPPCKECH | TVEKSLSRADCS |
| | KCPAPNLEGGPSVFIFPPNIKDVLMISLTPK | (SEQ ID NO: 28) |
| | VTCVVVDVSEDDPDVQISWFVNNVEVHTA | Note that glycine at position |
| | QTQTHREDYNSTIRVVSTLPIQHQDWMSG | 116 is optional and included |
| | KEFKCKVNNKDLPSPIERTISKIKGLVRAPQ | for improving expression levels. |
| | VYILPPPAEQLSRKDVSLTCLVVGFNPGDIS | |
| | VEWTSNGHTEENYKDTAPVLDSDGSYFIYS | |
| | KLNMKTSKWEKTDSFSCNVRHEGLKNYYL | |
| | KKTISRSPGK | |
| | (SEQ ID NO: 27) | |
| ACI-31- | EVQLVESGGDLVKPGGSLKLSCAASRFTFS | QLVLTQSSSASFSLGASAKLTCTLSSQHITY |
| 25B1-Ab2 | TYGMSWVRQTPDKRLEWVATISSGRSYTY | TIEWYQQQPLKPPKYVMVLEKDGSHSTGD |
| (murine | YADSVKGRFTISRDNAKNTLYLQMSSLKSE | GIPDRFSGSSSGADRYLSISNIQPEDEAVYIC |
| IgG2a with | DTAMYYCARNSNNQRDYYAMDYWGQGT | GVGDAIKEQFVYVFGGGTKVTVLGQPKSS |
| a lambda | SVTVSSAKTTAPSVYPLAPVCGDTTGSSVT | PSVTLFPPSSEELETNKATLVCTITDFYPGV |
| light | LGCLVKGYFPEPVTLTWNSGSLSSGVHTFP | VTVDWKVDGTPVTQGMETTQPSKQSNNK |
| chain) | AVLQSDLYTLSSSVTVTSSTWPSQSITCNVA | YMASSYLTLTARAWERHSSYSCQVTHEGH |
| | HPASSTKVDKKIEPRGPTIKPCPPCKCPAPN | TVEKSLSRADCS |
| | LLGGPSVFIFPPKIKDVLMISLSPIVTCVVVD | (SEQ ID NO: 39) |
| | VSEDDPDVQISWFVNNVEVHTAQTQTHRE | |
| | DYNSTLRVVSALPIQHQDWMSGKEFKCKV | |
| | NNKDLPAPIERTISKPKGSVRAPQVYVLPPP | |
| | EEEMTKKQVTLTCMVTDFMPEDIYVEWTN | |
| | NGKTELNYKNTEPVLDSDGSYFMYSKLRV | |
| | EKKNWVERNSYSCSVVHEGLHNHHTTKSF | |
| | SRTPGK | |
| | (SEQ ID NO: 38) | |
| ACI-31- | EVQLVESGGDLVKPGGSLKLSCAASRFTFS | QLVLTQSSSASFSLGASAKLTCTLSSQHITY |
| 25B1-Ab2 | TYGMSWVRQTPDKRLEWVATISSGRSYTY | TIEWYQQQPLKPPKYVMVLEKDGSHSTGD |
| (human | YADSVKGRFTISRDNAKNTLYLQMSSLKSE | GIPDRFSGSSSGADRYLSISNIQPEDEAVYIC |
| IgG1/ | DTAMYYCARNSNNQRDYYAMDYWGQGT | GVGDAIKEQFVYVFGGGTKVTVLGQPKAN |
| Lambda | SVTVSSASTKGPSVFPLAPSSKSTSGGTAAL | PTVTLFPPSSEELQANKATLVCLISDFYPGA |
| isotype) | GCLVKDYFPEPVTVSWNSGALTSGVHTFP | VTVAWKADGSPVKAGVETTKPSKQSNNK |
| | AVLQSSGLYSLSSVVTVPSSSLGTQTYICNV | YAASSYLSLTPEQWKSHRSYSCQVTHEGST |
| | NHKPSNTKVDKRVEPKSCDKTHTCPPCPAP | VEKTVAPTECS |
| | ELLGGPSVFLFPPKPKDTLMISRTPEVTCVV | (SEQ ID NO: 59) |
| | VDVSHEDPEVKFNWYVDGVEVHNAKTKP | |
| | REEQYNSTYRVVSVLTVLHQDWLNGKEY | |
| | KCKVSNKALPAPIEKTISKAKGQPREPQVY | |
| | TLPPSREEMTKNQVSLTCLVKGFYPSDIAV | |
| | EWESNGQPENNYKTTPPVLDSDGSFFLYSK | |
| | LTVDKSRWQQGNVFSCSVMHEALHNHYT | |
| | QKSLSLSPGK | |
| | (SEQ ID NO: 58) | |
| ACI-31- | QVQLQQSGPELVKPGASVKISCKASGYAFS | DIVLTQSPASLAVSLGQRATISCRASKSVDN |
| 30C11- | SSWLNWVKQRPGEGLEWIGRIYPGDGDIN | YGKSFMHWFQQKPGQPPKLLIYRASNLES |
| Ab1 | YNGKFKGKATLTADKSSSTAYMQLSSLTS | GIPARFSGSGSRTDFTLTINPVEADDVATYY |
| (murine | DDSAVYFCTKRGRYALDYWGQGTSVTVSS | CQQSNEDPYTFGGGTKLEMKRADAAPTVS |
| IgG2b with | AKTTPPSVYPLAPGCGDTTGSSVTLGCLVK | IFPPSSEQLTSGGASVVCFLNNFYPKDINVK |
| a kappa | GYFPESVTVTWNSGSLSSSVHTFPALLQSG | WKIDGSERQNGVLNSWTDQDSKDSTYSMS |
| light | LYTMSSSVTVPSSTWPSQTVTCSVAHPASS | STLTLTKDEYERHNSYTCEATHKTSTSPIVK |
| chain) | TTVDKKLEPSGPISTINPCPPCKECHKCPAP | SFNRNEC |
| | NLEGGPSVFIFPPNIKDVLMISLTPKVTCVV | (SEQ ID NO: 49) |
| | VDVSEDDPDVQISWFVNNVEVHTAQTQTH | |
| | REDYNSTIRVVSTLPIQHQDWMSGKEFKCK | |
| | VNNKDLPSPIERTISKIKGLVRAPQVYILPPP | |
| | AEQLSRKDVSLTCLVVGFNPGDISVEWTSN | |
| | GHTEENYKDTAPVLDSDGSYFIYSKLNMK | |
| | TSKWEKTDSFSCNVRHEGLKNYYLKKTISR | |
| | SPGK | |
| | (SEQ ID NO: 48) | |
| ACI-31- | QVQLQQSGPELVKPGASVKISCKASGYAFS | DIVLTQSPASLAVSLGQRATISCRASKSVDN |
| 30C11- | SSWLNWVKQRPGEGLEWIGRIYPGDGDIN | YGKSFMHWFQQKPGQPPKLLIYRASNLES |
| Ab1 | YNGKFKGKATLTADKSSSTAYMQLSSLTS | GIPARFSGSGSRTDFTLTINPVEADDVATYY |
| (murine | DDSAVYFCTKRGRYALDYWGQGTSVTVSS | CQQSNEDPYTFGGGTKLEMKRADAAPTVS |
| IgG2a with | AKTTAPSVYPLAPVCGDTTGSSVTLGCLVK | IFPPSSEQLTSGGASVVCFLNNFYPKDINVK |
| a kappa | GYFPEPVTLTWNSGSLSSGVHTFPAVLQSD | WKIDGSERQNGVLNSWTDQDSKDSTYSMS |
| light | LYTLSSSVTVTSSTWPSQSITCNVAHPASST | STLTLTKDEYERHNSYTCEATHKTSTSPIVK |
| chain) | KVDKKIEPRGPTIKPCPPCKCPAPNLLGGPS | SFNRNEC |
| | VFIFPPKIKDVLMISLSPIVTCVVVDVSEDDP | (SEQ ID NO: 51) |
| | DVQISWFVNNVEVHTAQTQTHREDYNSTL | |
| | RVVSALPIQHQDWMSGKEFKCKVNNKDLP | |
| | APIERTISKPKGSVRAPQVYVLPPPEEEMTK | |
| | KQVTLTCMVTDFMPEDIYVEWTNNGKTEL | |
| | NYKNTEPVLDSDGSYFMYSKLRVEKKNW | |
| | VERNSYSCSVVHEGLHNHHTTKSFSRTPGK | |
| | (SEQ ID NO: 50) | |

TABLE 5
Amino acid sequences of Heavy Chain (HC) and
Light Chain (LC) murine IgG2a/kappa isotype
constant regions.

| Immuno- | |
|---|---|
| globulin | |
| region | Amino acid sequence |
| Murine | AKTTAPSVYPLAPVCGDTTGSSVTLGCLVK |
| IgG2a | GYFPEPVTLTWNSGSLSSGVHTFPAVLQSD |
| HC | LYTLSSSVTVTSSTWPSQSITCNVAHPASS |
| constant | TKVDKKIEPRGPTIKPCPPCKCPAPNLLGG |
| domains | PSVFIFPPKIKDVLMISLSPIVTCVVVDVS |
| | EDDPDVQISWFVNNVEVHTAQTQTHREDYN |
| | STLRVVSALPIQHQDWMSGKEFKCKVNNKD |
| | LPAPIERTISKPKGSVRAPQVYVLPPPEEE |
| | MTKKQVTLTCMVTDFMPEDIYVEWTNNGKT |
| | ELNYKNTEPVLDSDGSYFMYSKLRVEKKNW |
| | VERNSYSCSVVHEGLHNHHTTKSFSRTPGK |
| | (SEQ ID NO: 56) |
| Murine | RADAAPTVSIFPPSSEQLTSGGASVVCFLN |
| LC kappa | NFYPKDINVKWKIDGSERQNGVLNSWTDQD |
| domain | SKDSTYSMSSTLTLTKDEYERHNSYTCEAT |
| | HKTSTSPIVKSFNRNEC |
| | (SEQ ID NO: 57) |

Unless defined otherwise, all technical and scientific terms used herein have the same meanings as commonly understood by one of ordinary skill in the art to which this invention belongs. All publications and patents specifically mentioned herein are incorporated by reference in their entirety for all purposes in connection with the invention.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. Moreover, all aspects and embodiments of the invention described herein are considered to be broadly applicable and combinable with any and all other consistent embodiments, including those taken from other aspects of the invention (including in isolation) as appropriate.

### REFERENCES

Belichenko P V, Madani R, Rey-Bellet L, et al. An Anti-β-Amyloid Vaccine for Treating Cognitive Deficits in a Mouse Model of Down Syndrome. PLOS ONE. 2016; 11(3):e0152471.
Britt, W. G., 3rd, Hansen, A. M., Bhaskerrao, S., Larsen, J. P., Petersen, F., Dickson, A., . . . Kirsch, W. M. (2011). Mild cognitive impairment: prodromal Alzheimer's disease or something else? J Alzheimers Dis, 27(3), 543-551. doi:10.3233/JAD-2011-110740
Buchhave, P., Minthon, L., Zetterberg, H., Wallin, A. K., Blennow, K., & Hansson, O. (2012). Cerebrospinal fluid levels of beta-amyloid 1-42, but not of tau, are fully changed already 5 to 10 years before the onset of Alzheimer dementia. Arch Gen Psychiatry, 69(1), 98-106. doi:10.1001/archgenpsychiatry.2011.155
Calderon-Garciduenas, A. L., & Duyckaerts, C. (2017). Alzheimer disease. Handb Clin Neurol, 145, 325-337. doi:10.1016/B978-0-12-802395-2.00023-7
Gauthier, S., Reisberg, B., Zaudig, M., Petersen, R. C., Ritchie, K., Broich, K., . . . International Psychogeriatric Association Expert Conference on mild cognitive, i. (2006). Mild cognitive impairment. Lancet, 367(9518), 1262-1270. doi:10.1016/S0140-6736(06)68542-5
Given, A M, Whalen, P M, O'Brien, P J and Ray, C A (2012). Development and validation of an alpha fetoprotein immunoassay using Gyros technology. J Pham Biomed Anal., 64-65: 8-15. doi: 10.1016/j.jpba.2012.02.001
Graham et al., J. Gen. Virol. 36:59 (1977)
Head E, Powell D, Gold B T, Schmitt F A. Alzheimer's Disease in Down Syndrome. European journal of neurodegenerative disease. 2012; 1(3):353-364.
Knopman, D. S., DeKosky, S. T., Cummings, J. L., Chui, H., Corey-Bloom, J., Relkin, N., . . . Stevens, J. C. (2001). Practice parameter: diagnosis of dementia (an evidence-based review). Report of the Quality Standards Subcommittee of the American Academy of Neurology. Neurology, 56(9), 1143-1153. doi:10.1212/wnl.56.9.1143
Kiio and Soyeun Park. Nano-scientific Application of Atomic Force Microscopy in Pathology: from Molecules to Tissues. International Journal of Medical Sciences (2020); 17(7): 844-858. doi: 10.7150/ijms.41805
Mather, Biol. Reprod. 23:243-251 (1980)
Mather et al., Annals N.Y. Acad. Sci. 383:44-68 (1982)
Nabers A, Ollesch J, Schartner J, Kötting C, Genius J, Haußmann U, Klafki H, Wiltfang J, Gerwert K. J. An infrared sensor analysing label-free the secondary structure of the Abeta peptide in presence of complex fluids. Biophotonics. 2016 March; 9(3):224-34. doi: 10.1002/jbio.201400145. Epub 2015 Mar. 23. PMID: 25808829
Piraino F, Volpetti F, Watson C, Maerkl S J. A Digital-Analog Microfluidic Platform for Patient-Centric Multiplexed Biomarker Diagnostics of Ultralow Volume Samples. ACS Nano. 2016 Jan. 26; 10(1):1699-710. doi: 10.1021/acsnano.5b07939. Epub 2016 Jan. 12. PMID: 26741022
Robb, C., Udeh-Momoh, C., Wagenpfeil, S., Schope, J., Alexopoulos, P., Perneczky, R., & Alzheimer's Disease Neuroimaging, I. (2017). Biomarkers and Functional Decline in Prodromal Alzheimer's Disease. J Alzheimers Dis, 58(1), 69-78. doi:10.3233/JAD-161162
Shankar, G. M., Li, S., Mehta, T. H., Garcia-Munoz, A., Shepardson, N. E., Smith, I., . . . Selkoe, D. J. (2008). Amyloid-beta protein dimers isolated directly from Alzheimer's brains impair synaptic plasticity and memory. Nat Med, 14(8), 837-842. doi:10.1038/nm1782
Stech, M., Nikolaeva, O., Thoring, L. et al. Cell-free synthesis of functional antibodies using a coupled in vitro transcription-translation system based on CHO cell lysates. Sci Rep 7, 12030 (2017)
Urlaub et al., Proc. Natl. Acad. Sci. USA 77:4216 (1980)

1.-70. (canceled)

71. An amyloid-beta binding antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197.

72. The amyloid-beta binding antibody or antigen-binding fragment thereof of claim 71, wherein the antibody or the antigen-binding fragment thereof comprises:
a) a Heavy Chain Variable Region (VH) comprising the sequence of SEQ ID NO: 10 or a Heavy Chain Variable Region (VH) having at least 96%, 97%, 98%, or 99%, sequence identity to the amino acid sequence of SEQ ID NO: 10; or
b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99%, sequence identity to the amino acid sequence of SEQ ID NO: 30; or
c) a Heavy Chain Variable Region (VH) comprising the sequence of SEQ ID NO: 40 or a Heavy Chain Variable Region (VH) having at least 93%, 94%, 95%, 96%, 97%, 98%, or 99%, sequence identity to the amino acid sequence of SEQ ID NO: 40; or
d) a Heavy Chain Variable Region (VH) comprising the sequence of SEQ ID NO: 60 or a Heavy Chain Variable Region (VH) having at least 98%, or 99%, sequence identity to the amino acid sequence of SEQ ID NO: 60; or
e) a Heavy Chain Variable Region (VH) comprising the sequence of SEQ ID NO: 70 or a Heavy Chain Variable Region (VH) having at least 98%, or 99%, sequence identity to the amino acid sequence of SEQ ID NO: 70; or
f) a Heavy Chain Variable Region (VH) comprising the sequence of SEQ ID NO: 80 or a Heavy Chain Variable Region (VH) having at least 99%, sequence identity to the amino acid sequence of SEQ ID NO: 80; or
g) a Heavy Chain Variable Region (VH) comprising the sequence of SEQ ID NO: 90 or a Heavy Chain Variable Region (VH) having at least 97%, 98%, or 99%, sequence identity to the amino acid sequence of SEQ ID NO: 90; or
h) a Heavy Chain Variable Region (VH) comprising the sequence of SEQ ID NO: 100 or a Heavy Chain Variable Region (VH) having at least 99%, sequence identity to the amino acid sequence of SEQ ID NO: 100; or
i) a Heavy Chain Variable Region (VH) comprising the sequence of SEQ ID NO: 110 or a Heavy Chain Variable Region (VH) having at least 97%, 98%, or 99%, sequence identity to the amino acid sequence of SEQ ID NO: 110; or
j) a Heavy Chain Variable Region (VH) comprising the sequence of SEQ ID NO: 120 or a Heavy Chain Variable Region (VH) having at least 95%, 96%, 97%, 98%, or 99%, sequence identity to the amino acid sequence of SEQ ID NO: 120; or
k) a Heavy Chain Variable Region (VH) comprising the sequence of SEQ ID NO: 130 or a Heavy Chain Variable Region (VH) having at least 94%, 95%, 96%, 97%, 98%, or 99%, sequence identity to the amino acid sequence of SEQ ID NO: 130; or
l) a Heavy Chain Variable Region (VH) comprising the sequence of SEQ ID NO: 140 or a Heavy Chain Variable Region (VH) having at least 96%, 97%, 98%, or 99%, sequence identity to the amino acid sequence of SEQ ID NO: 140; or
m) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99%, sequence identity to the amino acid sequence of SEQ ID NO: 150; or
n) a Heavy Chain Variable Region (VH) comprising the sequence of SEQ ID NO: 160 or a Heavy Chain Variable Region (VH) having at least 97%, 98%, or 99%, sequence identity to the amino acid sequence of SEQ ID NO: 160; or
o) a Heavy Chain Variable Region (VH) comprising the sequence of SEQ ID NO: 170 or a Heavy Chain Variable Region (VH) having at least 92%, 93%, 94%, 95%, 96%, 97%, 98%, or 99%, sequence identity to the amino acid sequence of SEQ ID NO: 170; or
p) a Heavy Chain Variable Region (VH) comprising the sequence of SEQ ID NO: 180 or a Heavy Chain Variable Region (VH) having at least 94%, 95%, 96%, 97%, 98%, or 99%, sequence identity to the amino acid sequence of SEQ ID NO: 180.

73. The amyloid-beta binding antibody or antigen-binding fragment thereof of claim 71, wherein the antibody or the antigen-binding fragment thereof comprises:
a) a Heavy Chain Variable Region (VH) comprising the sequence of SEQ ID NO: 10 or a Heavy Chain Variable Region (VH) having at least 96%, 97%, 98%, or 99% sequence identity to the amino acid sequence of SEQ ID NO: 10; and a Light Chain Variable Region (VL) comprising the sequence of SEQ ID NO: 14; or
b) a Heavy Chain Variable Region (VH) comprising the sequence of SEQ ID NO: 30 or a Heavy Chain Variable Region (VH) having at least 91%, 92%, 93%, 94%, 95%, 96%, 97%, 98%, or 99% sequence identity to the amino acid sequence of SEQ ID NO: 30; and a Light Chain Variable Region (VL) comprising the sequence of SEQ ID NO: 34 or a Light Chain Variable Region (VL) having at least 96%, 97%, 98%, or 99% sequence identity to the amino acid sequence of SEQ ID NO: 34; or
c) a Heavy Chain Variable Region (VH) comprising the sequence of SEQ ID NO: 40 or a Heavy Chain Variable Region (VH) having at least 93%, 94%, 95%, 96%, 97%, 98%, or 99% sequence identity to the amino acid sequence of SEQ ID NO: 40; and a Light Chain Variable Region (VL) comprising the sequence of SEQ ID NO: 44 or a Light Chain Variable Region (VL) having at least 97%, 98%, or 99% sequence identity to the amino acid sequence of SEQ ID NO: 44; or
d) a Heavy Chain Variable Region (VH) comprising the sequence of SEQ ID NO: 60 or a Heavy Chain Variable Region (VH) having at least 98%, or 99% sequence identity to the amino acid sequence of SEQ ID NO: 60; and a Light Chain Variable Region (VL) comprising the sequence of SEQ ID NO: 64 or a Light Chain Variable Region (VL) having at least 98%, or 99% sequence identity to the amino acid sequence of SEQ ID NO: 64; or
e) a Heavy Chain Variable Region (VH) comprising the sequence of SEQ ID NO: 70 or a Heavy Chain Variable Region (VH) having at least 98%, or 99% sequence identity to the amino acid sequence of SEQ ID NO: 70; and a Light Chain Variable Region (VL) comprising the sequence of SEQ ID NO: 74 or a Light Chain Variable Region (VL) having at least 99% sequence identity to the amino acid sequence of SEQ ID NO: 74; or a Heavy Chain Variable Region (VH) comprising the sequence of SEQ ID NO: 80 or a Heavy Chain Variable Region (VH) having at least 99% sequence identity to the amino acid sequence of SEQ ID NO: 80; and a Light Chain Variable Region (VL) comprising the sequence of SEQ ID NO: 84; or
f) a Heavy Chain Variable Region (VH) comprising the sequence of SEQ ID NO: 90 or a Heavy Chain Variable Region (VH) having at least 97%, 98%, or 99% sequence identity to the amino acid sequence of SEQ ID NO: 90; and a Light Chain Variable Region (VL) comprising the sequence of SEQ ID NO: 94 or a Light Chain Variable Region (VL) having at least 98%, or 99% sequence identity to the amino acid sequence of SEQ ID NO: 94; or
g) a Heavy Chain Variable Region (VH) comprising the sequence of SEQ ID NO: 100 or a Heavy Chain Variable Region (VH) having at least 99% sequence identity to the amino acid sequence of SEQ ID NO: 100; and a Light Chain Variable Region (VL) comprising the sequence of SEQ ID NO: 104; or
h) a Heavy Chain Variable Region (VH) comprising the sequence of SEQ ID NO: 110 or a Heavy Chain Variable Region (VH) having at least 97%, 98%, or 99% sequence identity to the amino acid sequence of SEQ ID NO: 110; and a Light Chain Variable Region (VL) comprising the sequence of SEQ ID NO: 114 or a Light Chain Variable Region (VL) having at least 98%, or 99% sequence identity to the amino acid sequence of SEQ ID NO: 114; or
i) a Heavy Chain Variable Region (VH) comprising the sequence of SEQ ID NO: 120 or a Heavy Chain Variable Region (VH) having at least 95%, 96%, 97%, 98%, or 99% sequence identity to the amino acid sequence of SEQ ID NO: 120; and a Light Chain Variable Region (VL) comprising the sequence of SEQ ID NO: 124 or a Light Chain Variable Region (VL) having at least 99% sequence identity to the amino acid sequence of SEQ ID NO: 124; or
j) a Heavy Chain Variable Region (VH) comprising the sequence of SEQ ID NO: 130 or a Heavy Chain Variable Region (VH) having at least 94%, 95%, 96%, 97%, 98%, or 99% sequence identity to the amino acid sequence of SEQ ID NO: 130; and a Light Chain Variable Region (VL) comprising the sequence of SEQ ID NO: 134; or
k) a Heavy Chain Variable Region (VH) comprising the sequence of SEQ ID NO: 140 or a Heavy Chain Variable Region (VH) having at least 96%, 97%, 98%, or 99% sequence identity to the amino acid sequence of SEQ ID NO: 140; and a Light Chain Variable Region (VL) comprising the sequence of SEQ ID NO: 144; or
l) a Heavy Chain Variable Region (VH) comprising the sequence of SEQ ID NO: 150 or a Heavy Chain Variable Region (VH) having at least 90%, 91%, 92%, 93%, 94%, 95%, 96%, 97%, 98%, or 99% sequence identity to the amino acid sequence of SEQ ID NO: 150; and a Light Chain Variable Region (VL) comprising the sequence of SEQ ID NO: 154 or a Light Chain Variable Region (VL) having at least 98%, or 99% sequence identity to the amino acid sequence of SEQ ID NO: 154; or
m) a Heavy Chain Variable Region (VH) comprising the sequence of SEQ ID NO: 160 or a Heavy Chain Variable Region (VH) having at least 97%, 98%, or 99% sequence identity to the amino acid sequence of SEQ ID NO: 160; and a Light Chain Variable Region (VL) comprising the sequence of SEQ ID NO: 164 or a Light Chain Variable Region (VL) having at least 99% sequence identity to the amino acid sequence of SEQ ID NO: 164; or
n) a Heavy Chain Variable Region (VH) comprising the sequence of SEQ ID NO: 170 or a Heavy Chain Variable Region (VH) having at least 92%, 93%, 94%, 95%, 96%, 97%, 98%, or 99% sequence identity to the amino acid sequence of SEQ ID NO: 170; and a Light Chain Variable Region (VL) comprising the sequence of SEQ ID NO: 174; or
o) a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 184; or
p) a Heavy Chain Variable Region (VH) comprising the sequence of SEQ ID NO: 180 or a Heavy Chain Variable Region (VH) having at least 94%, 95%, 96%, 97%, 98%, or 99% sequence identity to the amino acid sequence of SEQ ID NO: 180; and a Light Chain Variable Region (VL) comprising the sequence of SEQ ID NO: 194.

74. The amyloid-beta binding antibody or antigen-binding fragment thereof of claim 71, wherein the antibody comprises:
a) a Heavy Chain (HC) comprising the amino sequence of SEQ ID NO: 20 and a Light Chain (LC) comprising the amino sequence of SEQ ID NO: 21; or
b) a Heavy Chain (HC) comprising the amino sequence of SEQ ID NO: 22 and a Light Chain (LC) comprising the amino sequence of SEQ ID NO:23; or
c) a Heavy Chain (HC) comprising the amino sequence of SEQ ID NO: 24 and a Light Chain (LC) comprising the amino sequence of SEQ ID NO: 25; or
d) a Heavy Chain (HC) comprising the amino sequence of SEQ ID NO: 27 and a Light Chain (LC) comprising the amino sequence of SEQ ID NO: 28; or
e) a Heavy Chain (HC) comprising the amino sequence of SEQ ID NO: 38 and a Light Chain (LC) comprising the amino sequence of SEQ ID NO: 39; or
f) a Heavy Chain (HC) comprising the amino sequence of SEQ ID NO: 48 and a Light Chain (LC) comprising the amino sequence of SEQ ID NO: 49; or
g) a Heavy Chain (HC) comprising the amino sequence of SEQ ID NO: 50 and a Light Chain (LC) comprising the amino sequence of SEQ ID NO: 51; or
h) a Heavy Chain (HC) comprising the amino sequence of SEQ ID NO: 56 and a Light Chain (LC) comprising the amino sequence of SEQ ID NO: 57; or
i) a Heavy Chain (HC) comprising the amino sequence of SEQ ID NO: 58 and a Light Chain (LC) comprising the amino sequence of SEQ ID NO: 59.

75. The amyloid-beta binding antibody or antigen-binding fragment thereof of claim 71, which:
a) selectively binds to or captures any amyloid-beta peptides or species in solution independent of the conformational state of the amyloid-beta peptides or species;
b) binds to an epitope within amino acids residues 1-5 (SEQ ID NO: 7), 1-8 (SEQ ID NO: 2), 17-23 (SEQ ID NO: 9), 22-35 (SEQ ID NO: 29) or 26-34 (SEQ ID NO: 8) of SEQ ID NO: 1 or to an equivalent epitope in non-human amyloid-beta;
c) is a murine, a chimeric, a humanized or a human antibody or an antigen-binding fragment thereof;
d) is an IgM, IgG1, IgG2, IgG2a, IgG2b, IgG3 or IgG4 antibody or antigen-binding fragment thereof; and/or
e) is conjugated to another molecule, in particular a detectable label.

76. A method of detecting or quantifying amyloid-beta or amyloid-beta aggregates, including plaques, in a biological sample, such as a sample obtained from a subject, the method comprising contacting the sample with the amyloid-beta binding antibody or antigen-binding fragment thereof of claim 71 and detecting binding of the antibody or antigen-binding fragment thereof in order to detect amyloid-beta or amyloid-beta aggregates in the sample, or quantifying amyloid-beta or amyloid-beta aggregates in a sample based on the level of binding of the antibody or antigen-binding fragment thereof to amyloid-beta or amyloid-beta aggregates.

77. A method for diagnosing a disease, disorder and/or condition associated with amyloid-beta comprising contacting a sample obtained from a subject with the amyloid-beta binding antibody or antigen-binding fragment thereof of claim 71 and detecting binding of the antibody or antigen-binding fragment thereof in order to detect amyloid-beta in the sample or quantifying amyloid-beta in a sample based on the level of binding of the antibody or antigen-binding fragment thereof to amyloid-beta, wherein higher levels of amyloid-beta in the sample compared with a control level based on healthy subjects are indicative of a disease, disorder or condition associated with amyloid-beta or
wherein similar or higher levels of amyloid-beta in the sample compared with a diseased control level are indicative of a disease, disorder or condition associated with amyloid-beta,
the method optionally further comprising classifying the disease, disorder or condition associated with amyloid-beta.

78. A method for monitoring a disease, disorder or condition associated with amyloid-beta at two or more time points using samples from a subject comprising contacting the samples with an amyloid-beta binding antibody or antigen-binding fragment thereof of claim 71, wherein;
a. higher levels of amyloid-beta in the later sample compared with one or more earlier samples are indicative of progression of a disease, disorder or condition associated with amyloid-beta; or
b. lower levels of amyloid-beta in the later sample compared with one or more earlier samples are indicative of regression of a disease, disorder or condition associated with amyloid-beta; or
c. no significant change of levels of amyloid-beta in the later sample compared with one or more earlier samples are indicative of lack of progression of a disease, disorder or condition associated with amyloid-beta.

79. A method for selecting a therapy for treatment of a disease, disorder or condition associated with amyloid-beta comprising contacting samples taken before and after treatment with the therapy with an amyloid-beta binding antibody or antigen-binding fragment thereof of claim 71, wherein;
a. lower levels of amyloid-beta in the sample taken after treatment compared with the sample taken before treatment are indicative of successful treatment of a disease, disorder or condition associated with amyloid-beta and thus the therapy is selected for treatment;
b. no significant change of levels of amyloid-beta in the sample taken after treatment compared with the sample taken before treatment are indicative of successful treatment of a disease, disorder or condition associated with amyloid-beta and thus the therapy is selected for treatment;
c. a decline in the rate of increase of levels of amyloid-beta between samples taken during treatment compared with samples taken before treatment are indicative of successful treatment of a disease, disorder or condition associated with amyloid-beta and thus the therapy is selected for treatment;
d. higher levels of amyloid-beta in the sample taken after treatment compared with the sample taken before treatment are indicative of unsuccessful treatment of a disease, disorder or condition associated with amyloid-beta and thus the therapy is not selected for treatment; or
e. no decline in the rate of increase of levels of amyloid-beta between samples taken during treatment compared with samples taken before treatment are indicative of unsuccessful treatment of a disease, disorder or condition associated with amyloid-beta and thus the therapy is not selected for treatment.

80. A method for assessing a candidate therapy for a disease, disorder or condition associated with amyloid-beta, the method comprising, following treatment of one or more subjects, contacting samples from the one or more treated subjects with an antibody or antigen-binding fragment of claim 71, wherein lower levels of amyloid-beta in the samples compared with levels in corresponding samples from subjects not treated with the therapy are indicative of successful treatment of a disease, disorder or condition associated with amyloid-beta, optionally performed at multiple time points in matched samples between the treatment and placebo groups in order to monitor the effectiveness of the candidate therapy over a defined time period, optionally comprising contacting samples from the one or more treated subjects and the subjects not treated with the therapy with the antibody or antigen-binding fragment prior to treatment, with the therapy or placebo respectively, to determine base levels of amyloid-beta.

81. The method according to claim 77 wherein the disease, disorder or condition associated with amyloid-beta is selected from the group consisting of Alzheimer's Disease (AD), mild cognitive impairment (MCI), Down syndrome (DS), Down syndrome-related Alzheimer's Disease, cardiac amyloidosis, cerebral amyloid angiopathy (CAA), multiple sclerosis, Parkinson's disease, Parkinson's Disease with Dementia (PDD), Lewy body dementia, ALS (amyotrophic lateral sclerosis), Adult Onset Diabetes, inclusion body myositis (IBM), ocular amyloidosis, glaucoma, macular degeneration, lattice dystrophy, optic neuritis, Myotonic dystrophy and hepatic dysfunction or failure.

82. A diagnostic composition comprising:
(a) the amyloid-beta binding antibody or antigen-binding fragment thereof of claim 71 and an acceptable carrier or excipient, or
(b) at least two amyloid-beta binding antibodies or antigen-binding fragments thereof of claim 71 and an acceptable carrier or excipient, or
(c) a first amyloid-beta binding antibody or antigen-binding fragment thereof according to claim 71 and a second amyloid-beta binding antibody or antigen-binding fragment thereof that is not an antibody according to claim 1, and an acceptable carrier and/or excipient.

83. A nucleic acid encoding the amyloid-beta binding antibody or antigen-binding fragment thereof of claim 71 or comprising a nucleotide sequence as provided in SEQ ID NO: 18, SEQ ID NO: 19, SEQ ID NO: 52, SEQ ID NO: 53, SEQ ID NO: 54, SEQ ID NO: 55, SEQ ID NO: 68, SEQ ID NO: 69, SEQ ID NO: 78, SEQ ID NO: 79, SEQ ID NO: 88, SEQ ID NO: 89, SEQ ID NO: 98, SEQ ID NO: 99, SEQ ID NO: 108, SEQ ID NO: 109, SEQ ID NO: 118, SEQ ID NO: 119, SEQ ID NO: 128, SEQ ID NO: 129, SEQ ID NO: 138, SEQ ID NO: 139, SEQ ID NO: 148, SEQ ID NO: 149, SEQ ID NO: 158, SEQ ID NO: 159, SEQ ID NO: 168, SEQ ID NO: 169, SEQ ID NO: 178, SEQ ID NO: 179, SEQ ID NO: 188, SEQ ID NO: 189 or SEQ ID NO: 199.

84. A recombinant vector comprising the nucleic acid of claim 83.

85. A host cell that comprises the nucleic acid of claim 83 or a recombinant vector comprising the nucleic acid, or that is an isolated host cell and expresses an amyloid-beta binding antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197.

86. A method for producing an amyloid-beta binding antibody or antigen-binding fragment thereof, comprising the steps of:
a. culturing an isolated host cell that expresses the amyloid-beta binding antibody or antigen-binding fragment of claim 71 under conditions suitable for producing the amyloid-beta binding antibody or antigen-binding fragment thereof, and
b. recovering the amyloid-beta binding antibody or antigen-binding fragment thereof.

87. A kit for detecting or quantifying amyloid-beta or for diagnosis of a disease, disorder or condition associated with amyloid-beta, comprising the amyloid-beta binding antibody or antigen-binding fragment thereof according to claim 71 and a container.

88. A method of detecting amyloid-beta aggregates, including plaques, in a subject, the method comprising contacting the subject with the amyloid-beta binding antibody or antigen-binding fragment thereof of claim 71 and detecting binding of the antibody or antigen-binding fragment thereof in order to detect amyloid-beta aggregates, including plaques, in the subject.

89. The diagnostic composition of claim 82, wherein two amyloid-beta binding antibodies or antigen-binding fragments selectively bind to or capture any amyloid-beta peptides or species in solution independently of the conformational state of the amyloid-beta peptides or species, and wherein at least one of the two amyloid-beta binding antibodies or antigen-binding fragments does not display any cross-reactivity to soluble APPalpha.

90. The diagnostic composition according to claim 82, wherein at least one, or two, of the amyloid-beta binding antibody or antigen-binding fragments are selected from:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; and/or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; and/or
c) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

91. A method of detecting amyloid-beta in a sample obtained from a subject, the method comprising the step of capturing amyloid beta with a first amyloid-beta binding antibody or fragment thereof and the step of detecting captured amyloid-beta in the sample with a second amyloid-beta binding antibody or fragment thereof, wherein:
a) each amyloid-beta binding antibody or antigen-binding fragment thereof is selected from the antibodies or antigen-binding fragments according to claim 71, or
b) one amyloid-beta binding antibody or antigen-binding fragment thereof according to claim 71 and the other antibody an amyloid-beta binding antibody or antigen-binding fragment thereof different to the antibody according to claim 71, or
c) the first and second amyloid-beta binding antibodies or antigen-binding fragments thereof are selected from claim 71, and wherein at least one of the first and second antibody or antigen-binding fragment thereof does not display any cross-reactivity to soluble APPalpha, or
d) each amyloid-beta binding antibody or antigen-binding fragment thereof is independently selected from:
i) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
ii) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
iii) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157.

92. A diagnostic composition comprising:
a. a first amyloid-beta binding antibody or antigen-binding fragment thereof that selectively binds to any amyloid-beta peptides or species in solution independently of the conformational state of the amyloid-beta peptides or species, and
b. a second amyloid-beta binding antibody or antigen-binding fragment thereof that selectively binds to any amyloid-beta peptides or species in solution independently of the conformational state of the amyloid-beta peptides or species,
wherein at least one, or both, of the first and second antibodies displays no cross-reactivity to soluble amyloid precursor protein (APP), in particular with no cross-reactivity to soluble APP alpha.

93. The diagnostic composition of claim 92 wherein
(a) at least one of the first and second antibody or antigen-binding fragment thereof that displays no cross-reactivity to soluble amyloid precursor protein (APP), in particular with no cross-reactivity to soluble APP alpha is an amyloid-beta binding antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197;
(b) the antibody or antigen-binding fragment thereof that displays no cross-reactivity to soluble amyloid precursor protein (APP), in particular with no cross-reactivity to soluble APP alpha comprises:
i. a Heavy Chain Variable Region (VH) comprising the sequence of SEQ ID NO: 30 and a Light Chain Variable Region (VL) comprising the sequence of SEQ ID NO: 34; or
ii. a Heavy Chain Variable Region (VH) comprising the sequence of SEQ ID NO: 150 and a Light Chain Variable Region (VL) comprising the sequence of SEQ ID NO: 154; or
(c) one of the first and second antibody or antigen-binding fragment thereof displays cross-reactivity (which may be low or high) to soluble amyloid precursor protein (APP), optionally wherein the antibody or antigen-binding fragment thereof displaying cross-reactivity (which may be low or high) to soluble amyloid precursor protein (APP) is an antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197, optionally wherein the antibody or antigen-binding fragment thereof displaying cross-reactivity (which may be low or high) to soluble amyloid precursor protein (APP) comprises a Heavy Chain Variable Region (VH) comprising the sequence of SEQ ID NO: 10 and a Light Chain Variable Region (VL) comprising the sequence of SEQ ID NO: 14.

94. A method of detecting amyloid-beta in a sample obtained from a subject, the method comprising:
a. capturing amyloid beta with an amyloid-beta binding antibody or antigen-binding fragment thereof that selectively binds to any amyloid-beta peptides or species in solution independently of the conformational state of the amyloid-beta peptides or species, and
b. detecting captured amyloid beta with an amyloid-beta binding antibody or antigen-binding fragment thereof that selectively binds to any amyloid-beta peptides or species in solution independently of the conformational state of the amyloid-beta peptides or species and displays no, low or high cross-reactivity to soluble amyloid precursor protein (APP), in particular with no, low or high cross-reactivity to soluble APP alpha,
wherein at least one, or both, of the capture and detection antibody or antigen-binding fragment thereof displays no cross-reactivity to soluble amyloid precursor protein (APP), in particular with no cross-reactivity to soluble APP alpha.

95. The method of claim 94 wherein:
(a) at least one of the capture and detection antibody or antigen-binding fragment thereof that displays no cross-reactivity to soluble amyloid precursor protein (APP), in particular with no cross-reactivity to soluble APP alpha is an antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197; or
(b) one of the capture and detection antibody or antigen-binding fragment thereof displays cross-reactivity (which may be low or high) to soluble amyloid precursor protein (APP), optionally wherein the antibody or antigen-binding fragment thereof displaying cross-reactivity (which may be low or high) to soluble amyloid precursor protein (APP) is an antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197.

96. A kit for detecting amyloid-beta in a sample obtained from a subject, the kit comprising:
a. a capture amyloid-beta binding antibody or antigen-binding fragment thereof that selectively binds to any amyloid-beta peptides or species in solution independently of the conformational state of the amyloid-beta peptides or species; and
b. a detection amyloid-beta binding antibody or antigen-binding fragment thereof that selectively binds to any amyloid-beta peptides or species in solution independently of the conformational state of the amyloid-beta peptides or species
wherein at least one of the capture and detection antibody or antigen-binding fragment thereof displays no cross-reactivity to soluble amyloid precursor protein (APP), in particular with no cross-reactivity to soluble APP alpha.

97. The kit of claim 96, wherein:
(a) at least one of the capture and detection antibody or antigen-binding fragment thereof that displays no cross-reactivity to soluble amyloid precursor protein (APP), in particular with no cross-reactivity to soluble APP alpha is an antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197; or,
(b) one of the capture and detection antibody or antigen-binding fragment thereof displays cross-reactivity (which may be low or high) to soluble amyloid precursor protein (APP), optionally wherein the antibody or antigen-binding fragment thereof displaying cross-reactivity (which may be low or high) to soluble amyloid precursor protein (APP) is an antibody or antigen-binding fragment thereof comprising:
a) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 13; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 17; or
b) a VH-CDR1 comprising the amino acid sequence of SEQ ID NO: 31; a VH-CDR2 comprising the amino acid sequence of SEQ ID NO: 32; a VH-CDR3 comprising the amino acid sequence of SEQ ID NO: 33; a VL-CDR1 comprising the amino acid sequence of SEQ ID NO: 35; a VL-CDR2 comprising the amino acid sequence of SEQ ID NO: 36; and a VL-CDR3 comprising the amino acid sequence of SEQ ID NO: 37; or
c) a VH-CDR1 comprising the amino acid sequence of SEQ ID NO: 41; a VH-CDR2 comprising the amino acid sequence of SEQ ID NO: 42; a VH-CDR3 comprising the amino acid sequence of SEQ ID NO: 43; a VL-CDR1 comprising the amino acid sequence of SEQ ID NO: 45; a VL-CDR2 comprising the amino acid sequence of SEQ ID NO: 46; and a VL-CDR3 comprising the amino acid sequence of SEQ ID NO: 47; or
d) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 63; a VL-CDR1 comprising the amino acid sequence of SEQ ID NO: 65; a VL-CDR2 comprising the amino acid sequence of SEQ ID NO: 16; and a VL-CDR3 comprising the amino acid sequence of SEQ ID NO: 17; or
e) a VH-CDR1 comprising the amino acid sequence of SEQ ID NO: 11; a VH-CDR2 comprising the amino acid sequence of SEQ ID NO: 12; a VH-CDR3 comprising the amino acid sequence of SEQ ID NO: 73; a VL-CDR1 comprising the amino acid sequence of SEQ ID NO: 75; a VL-CDR2 comprising the amino acid sequence of SEQ ID NO: 16; and a VL-CDR3 comprising the amino acid sequence of SEQ ID NO: 17; or
f) a VH-CDR1 comprising the amino acid sequence of SEQ ID NO: 61; a VH-CDR2 comprising the amino acid sequence of SEQ ID NO: 62; a VH-CDR3 comprising the amino acid sequence of SEQ ID NO: 83; a VL-CDR1 comprising the amino acid sequence of SEQ ID NO: 85; a VL-CDR2 comprising the amino acid sequence of SEQ ID NO: 16; and a VL-CDR3 comprising the amino acid sequence of SEQ ID NO: 87; or
g) a VH-CDR1 comprising the amino acid sequence of SEQ ID NO: 91; a VH-CDR2 comprising the amino acid sequence of SEQ ID NO: 92; a VH-CDR3 comprising the amino acid sequence of SEQ ID NO: 93; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 97; or
h) a VH-CDR1 comprising the amino acid sequence of SEQ ID NO: 101; a VH-CDR2 comprising the amino acid sequence of SEQ ID NO: 102; a VH-CDR3 comprising the amino acid sequence of SEQ ID NO: 103; a VL-CDR1 comprising the amino acid sequence of SEQ ID NO: 105; a VL-CDR2 comprising the amino acid sequence of SEQ ID NO: 106; and a VL-CDR3 comprising the amino acid sequence of SEQ ID NO: 107; or
i) a VH-CDR1 comprising the amino acid sequence of SEQ ID NO: 111; a VH-CDR2 comprising the amino acid sequence of SEQ ID NO: 112; a VH-CDR3 comprising the amino acid sequence of SEQ ID NO: 113; a VL-CDR1 comprising the amino acid sequence of SEQ ID NO: 115; a VL-CDR2 comprising the amino acid sequence of SEQ ID NO: 116; and a VL-CDR3 comprising the amino acid sequence of SEQ ID NO: 117; or
j) a VH-CDR1 comprising the amino acid sequence of SEQ ID NO: 121; a VH-CDR2 comprising the amino acid sequence of SEQ ID NO: 122; a VH-CDR3 comprising the amino acid sequence of SEQ ID NO: 123; a VL-CDR1 comprising the amino acid sequence of SEQ ID NO: 125; a VL-CDR2 comprising the amino acid sequence of SEQ ID NO: 126; and a VL-CDR3 comprising the amino acid sequence of SEQ ID NO: 127; or
k) a VH-CDR1 comprising the amino acid sequence of SEQ ID NO: 131; a VH-CDR2 comprising the amino acid sequence of SEQ ID NO: 132; a VH-CDR3 comprising the amino acid sequence of SEQ ID NO: 133; a VL-CDR1 comprising the amino acid sequence of SEQ ID NO: 135; a VL-CDR2 comprising the amino acid sequence of SEQ ID NO: 16; and a VL-CDR3 comprising the amino acid sequence of SEQ ID NO: 137; or
l) a VH-CDR1 comprising the amino acid sequence of SEQ ID NO: 141; a VH-CDR2 comprising the amino acid sequence of SEQ ID NO: 142; a VH-CDR3 comprising the amino acid sequence of SEQ ID NO: 133; a VL-CDR1 comprising the amino acid sequence of SEQ ID NO: 15; a VL-CDR2 comprising the amino acid sequence of SEQ ID NO: 16; and a VL-CDR3 comprising the amino acid sequence of SEQ ID NO: 137; or
m) a VH-CDR1 comprising the amino acid sequence of SEQ ID NO: 151; a VH-CDR2 comprising the amino acid sequence of SEQ ID NO: 152; a VH-CDR3 comprising the amino acid sequence of SEQ ID NO: 153; a VL-CDR1 comprising the amino acid sequence of SEQ ID NO: 155; a VL-CDR2 comprising the amino acid sequence of SEQ ID NO: 156; and a VL-CDR3 comprising the amino acid sequence of SEQ ID NO: 157; or
n) a VH-CDR1 comprising the amino acid sequence of SEQ ID NO: 161; a VH-CDR2 comprising the amino acid sequence of SEQ ID NO: 162; a VH-CDR3 comprising the amino acid sequence of SEQ ID NO: 163; a VL-CDR1 comprising the amino acid sequence of SEQ ID NO: 165; a VL-CDR2 comprising the amino acid sequence of SEQ ID NO: 166; and a VL-CDR3 comprising the amino acid sequence of SEQ ID NO: 167; or
o) a VH-CDR1 comprising the amino acid sequence of SEQ ID NO: 171; a VH-CDR2 comprising the amino acid sequence of SEQ ID NO: 172; a VH-CDR3 comprising the amino acid sequence of SEQ ID NO: 173; a VL-CDR1 comprising the amino acid sequence of SEQ ID NO: 175; a VL-CDR2 comprising the amino acid sequence of SEQ ID NO: 156; and a VL-CDR3 comprising the amino acid sequence of SEQ ID NO: 157; or
p) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 95; a VL-CDR2 comprising the amino acid sequence of SEQ ID NO: 96; and a VL-CDR3 comprising the amino acid sequence of SEQ ID NO: 187; or
q) a VH-CDR1 comprising the amino acid sequence of SEQ ID NO: 181; a VH-CDR2 comprising the amino acid sequence of SEQ ID NO: 182; a VH-CDR3 comprising the amino acid sequence of SEQ ID NO: 183; a VL-CDR1 comprising the amino acid sequence of SEQ ID NO: 195; a VL-CDR2 comprising the amino acid sequence of SEQ ID NO: 196; and a VL-CDR3 comprising the amino acid sequence of SEQ ID NO: 197.

98. The method of claim 76 wherein the sample is a body fluid or tissue sample, preferably wherein the body fluid or tissue sample is saliva, urine, nasal secretion, blood (including whole blood, plasma and serum, preferably plasma), brain or CSF sample, brain or ISF sample, more particularly blood, brain, CSF or ISF sample.

99. The diagnostic composition of claim 92 further comprising a sample, in particular a body fluid or tissue sample, preferably wherein the body fluid or tissue sample is saliva, urine, nasal secretion, blood (including whole blood, plasma and serum, preferably plasma), brain or CSF sample, brain or ISF sample, more particularly blood, brain, CSF or ISF sample.
